SEQUENCE LISTING (15 sequences)

SEQ ID NO: 1 - protein, 844 aa, unidentified
MAPTFQTALFFTIISLSFAAPNAKQVRWCAISDLEQKKCNDLVGSCNVPDITLVCVLRSSTEDCMTAIKDGQADAMFLDSGEVYEASKDPYNLKPIIAEPYSSNRDLQKCLKERQQALAKKMIGHYIPQCDEKGNYQPQQCHGSTGHCWCVNAMGEKISGTNTPPGQTRATCERHELPKCLKERQVALGGDEKVLGRFVPQCDEKGNYEPQQFHGSTGYSWCVNAIGEEIAGTKTPPGKIPATCQKHDLVTTCHYAVAMVKKSSAFQFNQLKGKRSCHSGVSKTDGWKALVTVLVEKKLLSWDGPAKESIQRAMSKFFSVSCIPGATQTNLCKQCKGEEGKNCKNSHDEPYYGNYGAFRCLKEDMGDVAFLRSTALSDEHSEVYELLCPDNTRKPLNKYKECNLGTVPAGTVVTRKISDKTEDINNFLMEAQKRQCKLFSSAHGKDLMFDDSTLQLALLSSEVDAFLYLGVKLFHAMKALTGDAHLPSKNKVRWCTINKLEKMKCDDWSAVSGGAIACTEASCPKGCVKQILKGEADAVKLEVQYMYEALMCGLLPAVEEYHNKDDFGPCKTPGSPYTDFGTLRAVALVKKSNKDINWNNIKGKKSCHTGVGDIAGWVIPVSLIRRQNDNSDIDSFFGESCAPGSDTKSNLCKLCIGDPKNSAANTKCSLSDKEAYYGNQGAFRCLVEKGDVAFVPHTVVFENTDGKNPAVWAKNLKSEDFELLCLDGSRAPVSNYKSCKLSGIPPPAIVTREESISDVVRIVANQQSLYGRKGFEKDMFQLFSSNKGNNLLFNDNTQCLITFDRQPKDIMEDYFGKPYYTTVYGASRSAMSSELISACTIKHC

SEQ ID NO: 2 - protein, 845 aa, Nanorana parkeri
MALTFHTALYFTIVGLSFAASDARHVQWCTISHLEQKKCNDLVGSCNVPDITLACVYRSSTENCMAAIKDGQADAMFLDSGDVYKASLDHYNLKPIIAEPYSLHRELTKCLKHRQESLGGDKMVKGRYIPQCDEKGNYHPVQCHASTGYCWCVNANGEKIEGTNTTPVQTPPTCPSQVLTKCLKERQEALGGKRIAIGRYIPQCDEQGNYRPMQCHGSTGYCWCVNAIGEKIEGTNTPPGNTQPTCQSHDWDTCHYAVAVVKNSSTFQFGQLKGKRSCHSGLSKTDGWNAPVNVFVEKKLLPWDGLAKGSIERAVSKFFSASCIPGATETNLCKQCIGEEEKKCKSSHDEPYYGDHGAFRCLQEDKGDVAFLKNTALPDEHSGVYELLCPDNTRKPLNKYKECNLGKVPADAVVTRKAGDKTKDINDFLLEAQKKKCKLFGSPHGKDLMFDDSTTHLAPLPSEIDAFFFLGVKWYNAMKALTEDVKLPSKNKVRWCTINKPEMMKCKDWAAVSGGAIACTEASCPEHCVKQILKGEADAVTLDVQYMYMALMCGLLPAVEEYPNKDDFHPCQIPGSTIKDFGTKRAVALVKKSNKDIKWNNLKGKKSCHTHVGDIPGWVIPAGLISNQNDNIDIESFFGESCAPGSDTNSKLCKLCIGDPENPKASTRCSLSDKEAYYGNEGAFRCLVEKGDVAFVPHTVVFANTDGKNPAEWAKDLKSEDFEILCLDGSRAPVTNYRGCNLSGLPPRAIVTREESVSDVVRILINQQSLYGRNGFEKDMFQMFSSAKGQNLLFNDETQCLIEFDRQPKDIMEDYFGVRYYTAVYSASRSAVPSELIPACTFKHC

SEQ ID NO: 3 - protein, 839 aa, Mantella aurantiaca
MALTFQTALYFTIIGLSFVTSSARDVRWCVISDLEQKKCNDLVGSCKVSGITLVCVHRSSTENCMTAIRDEQADAMFLDSGDVYKASMSPYNLKPIIAESYSSHKECLKKRQEALGGANVLGQFVPQCDEKGSYQPQQCYGSTGHCWCVNAIGEEIVGTKTQPGQTQATCERHDQTKCLKDRQKALAKKMIGGFVPQCDEKGNFKPRQCHGSTGYCWCTNANGEKIAATNTPPQQSPPTCERHDLDSCHYAVAVVKKSSIFQFNQLKGKRSCHSGVSKTDGWKAPVSVLVDKNLLSWDRAAKESIEKAVSKFFSDSCIPGATETNLCNQCMGEGGKKCMSSHDEPYYGDHGAFRCLKEDKGDVAFLKNTVLSDTHSEDYELLCPDNTRKPLNKYKECNLGKVPADAVVTRKSGDKIKDINDFLLEAQKKHCQLFSSAHGKDLMFDDSTTQLSLLPPEVDVFFFLGVQWLNTMKTLTADVKPPSKNKVRWCTVNNLEKMKCDDWSAVSGGAIECTEASSPEHCIKQIVKGEADAVTLDVQYMYEALKCGLLPAVEEYHNKDDFGPCKTPGSEIKDFGAKRAVALVKKSNKDINWHNLKGKKSCHTGVGDQAGWIIPVGLISRQIDNCNIDSFFSESCAPGSDTTSNFCKLCIGDPSNPMANTKCSLSDKEAYHGNEGAIRCLVEKGDVAFVHHTAVFENTDGKNPAEWAANLKSEDFEILCLDGSRAPVSNYMTCRLSGVPPRAIVTREESVSDVVRILINQQSLFGRNGFEKDMFQMFSSNKGQNLLFNDGTQCLIEFDRQPRGIMEDYFGKPYYNAVYNTSKCVLPSELTSACTFKHC

SEQ ID NO: 4 - protein, 912 aa, Dendrobates tinctorius
MRMTPQVLLWLCLLALSSAAPNIRNVRWCVTSDTEEQKCNDLVTSCHVDEILLTCVKKSSTEDCVRAISNGEADAISLDSKDVYKASLDPFNLKPIMTEAYSEREHTPCMRHRQSVLGGKNMKIGAFVPKCDEKGNYVPKQCHGSTGYCWCLNENGEEIEGTRTPPGNSGLTCENKANKPPCLKERQKLLSAKPSPAVFVPECDEKGNYRPQQSHVYTWCVDEYGEEVFGSRNFPGKPPKPCEASGETACIKERNKVLSVAEPLLGAFLPDCDENGYFSPLQFHGSTGYSWCVTKNGEEIKGTRTGPGQTPPTCEVSAPVTLHYAVAVVKKSSSFQLDQLKGKRSCHSAVGEAAGWVAPLNVLLKKKLLLWEEPEQKSIEKVASEIFSASCAPGAQEANLCEQCAGQEDQCTRGPGEPYYGDEGAFRCLRDGKGDVAFVEDTALTGQYSDNFELLCPDNTRRPLSQYKICNFGRIPRHSVVTRSTGDKMQDITEYLLQAQKKECKLFSSTHGKDLLFEDTTSALIALPSAMDTFLFLGPDLFNGMKTLNGARPPRVKQQIRWCPQSKNEHKKCEDWSSVSGGAIKCTEPSSALECVKKILKDEADAVNLDTAHTYTALKCGLLPSLDEYRNKDDLIPCQIPGADYTEFGSYRIVAIVKKKDKDITWNNLQGKKSCHTGVGDMMGWNIPVYLISKKTKNCDLGSYFSQSCAPGSPIDSNLCKLCIGDPQNTEANTKCSLSDKEAYYGNEGAVRCLVEKGDVAFVPHTAVFENTDGKNPALWAKDLKSADFELLCPDGSRAPVSNYKTCKLSGIANQVTVTRPESVKDVVRITQNQQSLYGRTGSLKDIFQMFSTSYGQNLLFSDRTQCLVEFDRMLDRDIMDDYFGKPFHKHLIRDNDCFPISALATACSFHH

SEQ ID NO: 5 - protein, 912 aa, Oophaga sylvatica
MRMTLQVLLCLCLLVLSSAAPNSRNVRWCVTSDTEEQKCNDLVTSCHVDEILLICVKKSSKEDCVRAISNGEADAISLDSKDVYKASLDPFNLKPIMTEAYSESEQTPCMRHRQSVLGGKKMIIGAFVPKCDEKGNYVPKQCHDSTGFCWCLNENGEEIEGTRTPPGNSGLTCENKANKPPCLKERQKLLSAKPSPAVFVPECDEKGNYRPEQSHVYSWCVDEYGEEVLGSRTFPGKPPKPCEASGETPCIKERNKVLSAAEPLPGAFVPDCDEKGYFSPLQFHGSTGHSWCVTKNGEEIKGTRTRPGQTPPTCDVPAPATLHYAVAVVKKSSSFQLDQLKGKRSCHSAVGEAAGWVAPLNVLLKKKFLLWEEPEQKSTEKVASEIFSASCAAGAQEANLCEQCAGQEDKCTRGPGEPYYGDEGAIRCLRDDKGDVAFVEDTALTGQYSDNFELLCPDNTRRPLSQYKICNFGSVPRHAVVTRSTGDKMKEITEYLLQAQKKECKLFSSTHGKDLLFEDTTSALIALPSGMDTFLFLGPDLFNGMKTLNGAHPPRVKQKIRWCPQNKNEKKKCDDWSSVSGGSIKCTEPSSALECIKMILKDEADAVNLDAEHAYTALKCGLLPSLEEYRNKDDLIVCQIPGAEYSEFGSYRIVALVKKTDKDITWNNLQGKKSCHTHVGDMIGWNIPVYLISKKTKNCDLGSYFSQSCAPGSPIDSNLCKLCIGDPQNTEANTKCSLSDKEAYYGNEGAVRCLVETGDVAFVPHTAVFENTDGKNPALWAKDLKSTDFELLCPDGSRAPVSDYKKCKLSGIANHVTVTRPESVKDVVRITQNQESLYGRTGSQKDIFQMFSSSYGQNLLFNDRTQCLFEFDRMLDRDIMDDYLGKPFHKHLMSDNDCLPKSALATACSFHH

SEQ ID NO: 6 - protein, 923 aa, Ranitomeya imitator
MRRSSYAASQEMRMTLRVLLCLCLWALSSAAPNTRNVRWCVTSDTEEQKCNDLVTSCHVDEILLICVKKSSTEDCVRAISNGEADAISLDSKDVYKASLDPFNLKPIMTEAYSESEHTPCMRHRQSVLGGKIMKIGAFVPKCDEKGNYVPKQCHGSTGYCWCLNENGEEIEGTRTPPGNPGLTCENKANKPPCLKERQKLLSAKPSPAVFVPECDEKGNYRPQQSHVYSWCVDEYGEEVFGSRSFPGKPPKPCEASGETPCIKERNKVLSAAEPLLGAFVPDCDEKGYFSPLQSHGSTGYSWCVTKNGDEIKGTRTGPGQSPPTCEVPAPVTLHYAVAVVKKSSSFHLDQLKGKRSCHSAVGEAAGWVAPLNVLLKKKLLLWEEPEPKTIEKVASEIFSASCAPGAQEANLCEQCAGQEDKCTRGPGEPYYGDEGAFRCLREDKGDVAFVEDTALTGQYSDNFELLCPDNTRRPLSQYKICNFGRIPRHAVVTRSSGDKMKDITEYLLQAQKKECKLFSSTHGKDLLFEDTTSALIALPSAMDTFLFLGPDLFNGMKTLNGARPPQVNQQIRWCPQSKNEKKKCDDWSSVSGGAIKCTEPSSGLECIKMILKGEAEAVNLDIEHSYIALKCGLLPSVDEYRNKDDLIPCQIPGSEYSEFGSYRIVALVKKTDKDLTWNNLQGKKSCHTRAGDMIGWNIPVYLISKKTKNCDLGSYFSQSCAPGSPIDSNLCKLCIGDPQNPQANTKCSISDKEAYYGHEGAIRCLVEKGDVAFVPHTAIFENTDGKNPALWAKDLKSTDFELLCPDGSRAPVNDYKKCKLSGIANQIAITRPESVKDVVRIIQNQQSLYGRTGSQKDIFQMFSSSYGQNLLFSDRTQCLVEFDRMLDRDIMDDYFGKPFHKHLLRDNDCLPKSALASACSFHH

SEQ ID NO: 7 - protein, 914 aa, Phyllobates terribilis
MRMTTAFQVLLCVCLLALSSAAPNARNVRWCVTSDAEEQKCNDLVTSCHVDEILLTCVKKYSTEDCVRAISNGEADAISLDSKDVYKASLHPFNLKPIVTEAYSEKEHTPCMRHRQSVLGGKKIKIGAFVPKCDEKGNYVPKQCHGSTGYCWCLNENGEEIEGTRTPPGTKHLTCEDTADKPPCLKKRQKLLSAKPSPAVFVPECDEKGNYRPEQSHVYTWCVDEYGEEVFGSRNFPGKPPKPCVASDEPLCIKERNKVLGTTEPLLGAFVPDCDEKGYFSPLQFHGSTGYSWCVTENGVEIKGTRTGPGQTPPTCEVPAPMTLNYAVAVVKKSSSFHLDQLKGKRSCHSAVGEAAGWVAPLNVLLKKKLLSWGGPEQKSIEKVASEIFSASCAPGAQEANLCEQCAGQEDKCTRGPGEPYYGDEGAFRCLRDDKGDVAFVEDTVLSGQYSDNFELLCPDNTRRPLSQYKICNFGRIPRHAVVTRTTGDKMKDITEYLVQGQKKECKLFSSTHGKDLLFEDTTTALITLPSAMDTFFFLGSELFKGMKTLEGTRPPRVHREIRWCPQNKNEKKKCDDWSSVSGGAIKCTEPSSALECIEMILKGEAEAVNLDAEHAYTALKCGLLPSVEEYRNKDDLIPCQVPGAEYSEFGGNRIVALVKKTDKDITWNDIQGKKSCHTHVGDMIGWNIPIYLIYKKTKNCDIGSFFSQSCAPGSTIDSNLCKLCIGDPQNPEANTKCSLSDKEAYYGNEGAIRCLVEKGDVAFVPHTAVYENTDGKNPALWAKDLKSTDFELLCLDGSRAPVSDYKKCKLSGIVNQVTVTRPESVKDVVRIIHNQESLYGRTGFQKDIFQMFSSSYGENLLFSDRTQCLFEFDRMLDRDIMEDYLGKPFHKYVMRDNECLPKSALATACSFHH

SEQ ID NO: 8 - protein, 919 aa, Epipedobates tricolor
MVSSRKLRMTSAFQLLLCLCLMVLSFAAPNARNVRWCVTSDAEEHKCNDLVNSCHVNDILLKCVKKSSTEDCVRAISNGEADTISLDSKDVYKASLHPFNLKPIMTEGYSEREHTPCMRHRQSVLEGKKMKIGAFVPKCDEKGNYAPKQCHGSSGYCWCLNENGEEIEGTRTPPGTKSLTCEDAANKPPCLKERQKLLSSKPAPTVFVPECDEKGNYRPQQCHIYCWCVDEYGEEVLFSRSFPGKPPKKCEPSGETLCLKQRNKVLSATEPLLGTFVPECDDKGYFTPQQFHGSTGYSWCVTKNGDEIPGTRTGPGQTPHICEVKAPVTLHYAVAVVKKSSSFQLDELKGKRSCHSAVGEAAGWVAPLKILLKKKLLSLEGAEVKSIEKAASEIFSASCAPGAHEAKLCEQCAGQEGKCHRGPGEPYYGDEGAFRCLRDNKGDVAFVEDTALSGQYSDNYELLCPDNTRRPLSQYKICNFGRVPRHAVVTRSSGDKMKDITEYLLEAQKKECKLFSSTHGKDLLFEDTTTGLIALPSAMDTFLFLGPELFHGMKTLDGAHPPSSQEIRWCPLTKNEKKKCDDWSSVSGGAIKCTEPSSGQKCIEKILKGEADVVTLGVEHAYTALKCGLHISFEEYRNKDDLIPCQVLEAEYTEFGSYRIVAVVKKTDKDITWNNLQGKKSCHTSVNDMVGWKIPHALLYKKTKSCDFGSYFSKSCAPGSAIDSNLCKLCIGDPQNTKSNTKCSLSDKEAYYGNDGAIRCLVEKGDVAFVPHTAIFENTDGKNPALWAKDLKSTDFELLCLDGSRAPVSDYKTCKLSGIANQVTVNRPERQADVLRIILNQQSLYGRTGSQKDIFQMFSSSYGQNLLFNDRTQCLFVFDRMLDRDIVDDYIGKAMQRDVLRDNECFPQTALTNACLFHH

SEQ ID NO: 9 - protein, 913 aa, Allobates femoralis
MRMTPALLGLLCLCLLVLSSAAPNSRTVRWCVTSDTEQQKCNDLVNSCHVDEIVLSCVKKSNTEDCVRAISTGEADAISLDSKDVYKASLDPFHLKPIMTEGYSERDHTPCMKHRQSVLGGKQLIIGAFVPKCDEKGNYAPKQCHGSSGYCWCLNENGEEIKGTRSRPGTKVLTCEDAANKPPCLKERQKLLSENPSPTVFVPECDEKGNYRPRQCHDYCWCVDEYGEEIFGSRTFPGKPPKACEASGETLCIKERNKVLSTAEPLRGAFLPECDEKGYFSPMQCHSSTGHCWCTTKDGEEIEGTRTGPGQSRPTCDIPAPGTVHYAVAVVKKSSNFQLDGLKGKRSCHSAVGESGGWVAPLNIFLKKNLLSWEGPEQKSIEKAASEFFSASCAPGAQESTLCEQCAGQEDKCKRGPGEPYYGDEGAIKCLKDDKGDVAFVEDTILSGQYSNDFELLCSDNTRRPLSQYKMCNFGKIPRHAVVTRSSGDKMEDITEYLLKAQKKECKLFGSAVGKDLLFEDTTTSLIVLPSAMDTFLYLGPELFSGMKTLEGAHPPSNQEIRWCPQSKNEKKKCDDWSSVSGGAIKCTEPSSALECIEKILKGEAEAVTLDGEHAYTALKCGLVPSLDEYRNKDDLVPCQIPGAEYSEFGSYRIVALVKKTDKDITWDNLQGKKSCHTSVGDMMGWTVPISLIYKKTKNCDIGSYFSQSCAPGSAIDSNLCELCIGDPGNPTANTKCSQNDKEAYYGNEGAIRCLVEKGDVTFVPHTAVYENTDGKNPALWAKDLKSTDFELLCPDGSRAPVSDYKRCKLSGIANQITVTRSESLKDVVRIILNQQSLYGRNGFQKDIFQLFSSVHGQNLLFSDRTQCLVEFDRMLDRDIMDDYFGKPFHKTVFRDNDCLPKSALATACSFHH

SEQ ID NO: 10 - protein, 944 aa, unidentified
MTPAFQVLLCLCLLGMSYAAPQARNVRWCVTSDAEEEKCNDLVNTCTVKEILLICVKKSSTEDCIRAISAGEADAISLDSEDIYQASLKPFHLKPIMTETYASKKNIKLNQSPCQKEHQHLLEGGPKCDETESTPCLRHRQRVLGARKPQIGEFVPECDEKGNYFPKQCYGTTGYCWCVDEHGDEIPVGRAKQGKVNISCEYAASEKPCMKEQRKALSGGQPLRGAFIPNCDEKGNYSPKQCHGSTGYCWCVDENGAEISGSRTPPGQQVPTCGSYVGATCIKDRYKVLGAGKPLPGAFVPDCDEKGDYRPQQCHGSTGHCWCVSKDGVEIQGTRAAPGQSPPTCEDPDLVKSHYAVAVVKKSSTLQFNHLKGKRSCHSAVGKTAGWIAPLYALYKKHFLLWDGSEEKSFEQAASEFFSASCAPGAKEENLCKQCAGQEDNCKSSPGEPYYGDEGALRCLREDKGDVAFVEHTALSGKDSDNFELLCPDNTRSPVSEHKDCNFGKVPRHAVVTRDTGDKSKDIIEYLLEAQKKECKLFSSTCGKDLMSGDTTTNLIALPSGMDAFLFLGPELFNAMKTLHGAPLPSKNEILWCTQNKNEKTKCDDWSAVSGGAIKCTEPLSADQCIEKILKREADAVTLHTEHMYTALKCGLVPAVEGYYNKDDFTPCKTDGDFYKDFGGTYVVALVKKSDKDITWNNLKGKKSCHTGVGHWTGWMIPVSQISKQTKNCDIGSYFSQSCAPGSAIDSNLCELCIGDQQNTKAQTQCSPSDKEAYYGGAGAIRCLVEKGDVAFVTDRDVFKNTDGNNPALWAKDLKSTDFELLCLDGSRAPVTDYKNCNLPGNPNLSVVSREENVNDVVRIFLNQQSLYGRNGSQKDKFQMFSSYGENLLFSDDAQCMIEFDMIKGRDLIDDVLGKHFSATMLRNNPCLPKSELRTACTFRAEKI

SEQ ID NO: 11 - protein, 1954 aa, Bufo bufo
MKHSSSSAQKEVRMTPAFQVLLCLCLLGMSYAAPQARNVRWCVTSDAEEEKCNDLVNSCPVKEILLICVKKSSIEDCMRAISAGEADAISLDSENIYKASLKPFHLKPIMTETYISKKDIKLTQGPCQKEHQHLLEGRQHKCEETESTPCLRHRQRVLGAKKPHIGEFMPECDEKGNYFPKQCYGTTGYCWCVDENGAERHVGRAKQGKVNISCEYTESQKPCMKERRKVLSGGQPLPGAFIPDCDEEGNYNPKQCHGSTGYCWCVDENGAEISGSRMPTGQSDPTCGTYAGATCIKDRYKVLGAGEALRGAFVPDCDEKGDYRSKQCHGSTGYCWCVSKYGVEIQGTRTAPGQSPTTCEIPGGEKPCMKERRKSLSGGQPLPGAFMPDCDEKGNYSPKQCHGSTGYCWCVNENGKEISGSRTPPGQQVPTCVASVLSAETSCIKERQKVLGAVKPILGAFVPDCDEKGEYRPKQCHGSTGYCWCVSKDGKEIQGTRAAPGQSPPTCQDTGEKLVLSAETSCIKERQKVLGAVKPILGAFVPDCDEKGEYRPKQCHGSTGYCWCVSKDGKEIQGTRAARGQSPPTCQDTGEKLVLSAETSCIKELQKVLGAVKPMVGAFVPDCDEKGEYRPKQCHGSTGYCWCVSKDGKEIQGTRVAPGQSPPTCQDTVLSAETSCIKERQKVLGAVKPILGAFLPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTVLSAETSCIKERQKVLGAVKPILGAFLPDCDEKGEYRPKQCHGSTGYCWCVSKDGKEIQGTRAAPGQSPPTCQDTVLSAETSCIKERQKVLGAVKPILGAFLPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTVLSAETSCIKERQKVLGAVKPILGAFLPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTVLSAETSCIKERQKVLGAVKPILGAFVPDCDEKGEYRPKQCHGSTGYCWCVSKDGKEIQGTRAAPGQSPPTCQDTGEKLVLSAETSCIKERQKVLGAVKPIVGAFVPDCDEKGEYRPKQCHGSTGHCWCVSKDGKEIQGTRVAPGQSPPTCQDTDPVTCHYAVAVVKKSSTLQFNQLKGKRSCHSAVGKTAGWIAPLYKLYKKNLLLWEKPEEKSFEKAASEFFSVSCAPGAKEENLCKQCAGKEDKCKRSPGELYYGDEGALRCLREDKGDVAFLEDIALSGQDLDNFELLCPDNTKSPLSEQKHCHFGKVPSHAVVTRSTGDKSKDIIEYLLEAQKKGCKLFSSTHGKDLMFEHTTTNVIALPSAMNTFLFLGPELFDAMKTLHGTPLPSKNEVRWCTQNQNEKTKCDDWSSVSGGAIKCTEPSSVQQCIEKILKHEADAVTLSAEHMYTALKCGLVPAVDEYHNKDDFAPCRTLGDIYTDFGTPRAVALVKKSNKDITWNNLKGKKSCHTGVGHMAGWVIPLSLISKQTNNCDLGSYFSQSCAPGSAIDSNLCKLCIGDPQNTKAQTQCSPSDKEAYYDSAGAIRCLVEKGDVAFLPHTAVFENTDGNNPALWAKDLKSTDFELLCPDGSRAPVTDYKNCKLLSISSPSVVTREESVSDVVRIVLSQQSLFGRKGFEKDMFQMFSSSNGKNLLFSDGTQCLLEFDRIIGRDIMEDYFGKPFHTAVHRDNQCLPTSELASACAFHHC

SEQ ID NO: 12 - protein, 1821 aa, Bufo gargarizans
MKHSSSSAQKEVRMTPAFQVLLCLCLLGMSYAAPQARNVRWCVTSNAEEKKCNDLVNSCTVKEIVLICVKKSSTEDCMRAISAGEADAISLDSENIYKASLNPFDLKPIMTETYPSQKEIKVNQGPCQKERQRQRERGRPLLGAFEPKCDEKGNYQPKQCHGSTGYCWCVNEEGKTIDGTKTPPGQKSVTCEDHQQSTPCLRHRQSVLGANKPQIGAFVPDCDEKGNYSPKQCFGSTGYCWCVDEHGDEIEGVRAKQGKVNITCEYTGGEKPCMKERRKSLSGGQPLPGAFMPDCDEKGNYSPKQCHGSTGYCWCVNENGKEISGSRTPPGQQVPTCGASVLSAETSCIKERQKVLGAATPILGAFVPDCDAKGDYRPKQCHGSTGHCWCVSKDGKEIQGTRTAPGQTPPTCEIPGGEKPCMKERRKSLSGGQPLPGAFMPDCDEKGNYSPKQCHGSTGYCWCVNENGKEISGSRTPPGQQVPTCGASVLSAETSCIKERQKVLGAEKPILGAFVPDCDEKGDYRPKQCHSSTGHCWCVSKDGKEIQGTRTAPGQTPPTCEIPGGEKPCMKERQKSLSGGQPLPGAFMPDCDEKGNYSPKQCHGSTGYCWCVNENGKEISGSRTPPGQQVPTCGASVLSADTSCIKERQKVLGAEKPILGAFEPDCDEKGDYRPKQCHGSTGHCWCVSKDGKEIQGTRTARGQSPPTCEIPVLSAETSCIKERQKVLGAATPILGAFVPDCDEKGDYRPKQCHGSTGHCWCVSKDGKEIQGTRAAPGQTPPTCEDRVLSAETSCIKERQKVLGAATPILGAFVPDCDEKGEYRPKQCHGSTGHCWCVSKDGKEIQGTRAGPGQTPPTCEDTAETSCIKERQKVLGAAKPILGAFVPDCDEKGEYRPKQCHGSTGHCWCVSKDGKEIQGTRAAPRQSPPTCEIPVLSAETSCIKEQQKVRAGKPILGAFVPDCDEKGDYRPKQCHGSTGHCWCVSKDGKEIQGTRAACGQSPPTCEIPAETSCIKERQKVLGAEKPILGAFVPDCDEKGEYRPKQCHGSTGHCWCVSKDGKEIQGTRAGPGQSPPTCEDTVLSAETSCIKERQKVLGAATPILGAFVPDCDEKGDYRPKQCHGSTGHCWCVSKDGKEIQGTRAGPGQSPPTCEIPAEKSCIKERQKVRSPRKPILGAFVPDCDEKGDYRPKQCHSSTGHCWCVSKDGKEIQGTRAARGQSPPTCEDPDPVTCHYAVAVVKKSSTLQFNQLKGKRSCHSAVGKTASWIAPLYKLYKKNLLLWEKPEEKSFEKAASEFFSVSCAPGAKEENLCKQCAGKEEKCKRSPGEPYYGDEGALRCLRDDKGDVAFVEHTALSGQDLDNFELLCPDNTRSPLSEHKHCHFGKVPRHAVVTKSTGDKSKDIIEYLLEAQKKGCKLFSSTHGKDLIFEHTTKNLIALPSAMNTFLFLGPELFDAMKTLHGAPLPSKNEVRWCTQNKKEKTKCDDWSSVSGGAIKCTEPSSVQQCIEKILKHEADAVTLSAEHMYTALKCGLVPAIDEYHNKDDLAPCKTLGDIYTDFGTPRAVALVKKSDKGITWNNLKGKKSCHTGVGHMAGWVIPLSLISKQTNNCDMGSYFSQSCAPGSAIDSNLCKLCIGDPQNTKAQTKCSPSDKEAYFGSAGAIRCLVEKGDVAFLPHTAVIENTDGNNPALWAKDLKSTDFELLCPDGSRAPVTDYKNCKLLSISSPSVVTREESVSDVVRIVLNQQSLFGQKGFEKDIFQMFSSSNGKNLLFSDGTQCLLEFDRIIGRDIMEDYFGKRFHTAVHRDNQCLPTSEFASACAFHHC

SEQ ID NO: 13 - protein, 764 aa, Engystomops pustulosus
MTAISHGEADAISLHGENIYKASFKPFNLIPIITESYHYQKGGRPLLGAFVPKCDEKGNYSPTQCHGGTGYCWCVTAEGKEIDGTKKPPGESPTCGEETKKTPCLNKREKALSGGQPLLGAFAPDCDEEGNFKPRQCYGSTGYCWCVDDNGVEISATRTPPGEKPPTCGATDPVTCHYAVAVVKKSSTFQFNQLKGKRSCHSAVGESAGWVAPMSALLDKKFLLWEGPAKKSFEKAASEFFSASCAPGAKEENLCKQCAGQQDKCKQSPGEPYYGDEGAFRCLREDKGDVAFVEHTVLSGQYSDNYELLCPDKTRKPLSQYEHCHFGKVPRPAVVTRHTGRKTKDITDYLLEAQKKECKLFSSTHGKNPPDTTTTLTSLPLAMDTFLFLGPELFNAMKRMHGEPLPSNKEVRWCPQSSDEKKKCDDWSAVSGGAIKCTEPFTTLQCIEKVLKGEAEAVTLNVENMYTALKCGLVPIVEEYHNKDDFNPCQSRGAQIKDFGTVKAVAVVKKRDKDINWNNLKGKKSCHTGVGHLAGWVVPVSLINKQSRTCDVESYFNQSCAPGSTTKSNLCKLCIGDPQNTKAKTKCSPNDKEAYYGNEGAIRCLAEKGDVAFVPHTAVFENTDGKNPALWAKDLKSTDFELLCPDGSRAPVSNYKNCKLAGIPPRAVVTRKDSASDVTKIIVNQQSLFGRKGFQKDIFQMFSSSNGQNLLFSDNTQCLIEFDRMIEKDIMEDYFGKTYYTAVHSDNQCFPPSALASACSFHHC

SEQ ID NO: 14 - protein, 852 aa, Eleutherodactylus coqui
MTPAFQLLLCLCLLGLSYTAPQGRNVRWCVTSDIEDKKCNDLANSCTVNEIHLKCVKKSSTLDCVKTISDGEADAISLDGEDIYKASLNPFNLKPIMVESHYHRAKNLPCLKERQEALGGGHPLPGAFVPNCDEKGNYNPKQCHGSTGYCWCVNKNGQKINGTETPPGQPSPTCEEEKSTPCLKHRQRLLGGNKPKIGAFVPKCDEKGDYIPKQCHGSTGQCWCVNTDGEEIAGTRTGPGKAPPTCEDAEPVTCHYAVAVVKKSSTLQFDQLKGKRSCHSAVGTAAGWAAPLNALMKRNLLVWKGPKEKTIEKAASEFFLSSCAPGAKETNLCKQCAGKEANCKHSSEEPYYGDEGAFKCLRDDKGDVAFVENSVLSEEHTDSFELLCPDNTRKQLSQYKDCNFGKVPRHAVVTRSSGDKIKDITEYLLGAQKKECELFSSTHGKRFLFEDTTTNLIALPSAMDTFLFLGPELFDAMKALHGEHPPSKNEVRWCTLSEKEKLKCDDWSSVSGGAIKCTEPSLAEECIEKVLKGEADVVSLSKKHMYAALTCGLIPAFEEYHNKDDFGPCQTPGTQYTDFGMTSNVALVKKKDQDITWNNLKGKKSCHPEVDDSAGWVDPLILIKKQTKSCDLGSFFSKSCAPGSAIDSNLCELCIGDSQNSKANTKCSRSDKEAYYGNDGAIRQKGDVAFVPHFAVSDNTDGKNPALWAKDLKSSDFELLCPDGSRAPVSDHKKCKLGGASIQTVVCREDRVSDVVRIIQNQQSLYGRKGFQKDIFQIFSSRHGRNILFHDDTQCLVDFDRVKEIDIMYDFFGKEHYDSIDFCLLIPSLIRRLGNRDLVIEMSLLVVFIPPS

SEQ ID NO: 15 - protein, 844 aa, Rana temporaria
MAPTFKAALFFTIISLSFAAPNAKQVRWCAISDLEQKKCNDLVGSCNVQDITLVCVLRTSTEDCLTAIKDGQADAMFLDSGDVYEASKDPYNLKPIIAEPYSSHKDLQKCLKEREQALAKKMIGHHIPQCDEKGNYQPQQCHGSTGQCWCVNGMGEKISGTNTPPGQTRATCERHELPKCLKERQVALGGDEIVLGRFVPQCDEKGNYEPQQFHGSTGYSWCVNEIGEEIAGTKTPPGKIPVTCEKHDLVTTCHYAVAMVKKSSTFQFNQLKGKRSCHSGVSKTDGWKALVTVLVEKKLLSWDGPAKESIQQAMSKFFSVSCIPGATQTNLCKQCKGEEGKNCKNSHDEPYYGNHGASRCLKEDKGDVAFLRSTALSDEDSEAYELLCPDNTRKPLNKYKECNLGTVPVGAVVTRKIGDKTVDINNFLMEAQKKQCKLFSSAHGKDLMFDDSTLQLALLSSGVDAFLYLGVKLFNTMKALTEDAHLSSKNKVRWCTINKLEKMKCDDWSAVSGGAIECTEASCPKCCIKQILKGEADAVKLEVQYMYEALTCGLLPAVEEYHNKDDFGPCKTPGSPYTDFGSPRAVALVKKSNKDINWNNIKGKKACLTGVGDIAGWVIPVSLIRRQNDNCDIDSFFGESCAPGSDTKSNLCKLCIGDPKNSVANTKCSLGDNEAYHGNKGAFRCLVEKGDVAFVPHTVVFENTDGKNPAEWAKKLKSEDFELLCLDGSRSPVSNYKSCKLSGIPPPAIVTREQSVTDVVRIVTNQQSLYGRKGFEKDIFQLFSSNKGKNLLFNDNTQCLIEFDRQPKDIMEDYFGKSYYTTVYGASRSAMSSELISACTVKHC

(19) United States
(12) Patent Application Publication
(10) Pub. No.: US 2026/0034194 A1
(43) Pub. Date: Feb. 5, 2026

(54) PHARMACEUTICAL COMPOSITIONS AND METHODS OF TREATING PSP

(71) Applicants: THE REGENTS OF THE UNIVERSITY OF CALIFORNIA, Oakland, CA (US); THE BOARD OF TRUSTEES OF THE LELAND STANFORD JUNIOR UNIVERSITY, Stanford, CA (US)

(72) Inventors: Daniel L. MINOR, Jr., Oakland, CA (US); Lauren O'CONNELL, Stanford, CA (US); Justin DU BOIS, Stanford, CA (US); Zhou CHEN, Oakland, CA (US); Sandra ZAKRZEWSKA, Oakland, CA (US); Fayal ABDEREMANE-ALI, Oakland, CA (US); Aurora ALVAREZ-BUYLLA, Stanford, CA (US)

(73) Assignees: THE REGENTS OF THE UNIVERSITY OF CALIFORNIA, Oakland, CA (US); THE BOARD OF TRUSTEES OF THE LELAND STANFORD JUNIOR UNIVERSITY, Stanford, CA (US)

(21) Appl. No.: 18/873,292

(22) PCT Filed: Jun. 9, 2023
(86) PCT No.: PCT/US2023/068244

§ 371 Date: Dec. 9, 2024

Related U.S. Application Data
(60) Provisional application No. 63/350,790, filed on Jun. 9, 2022.
(60) Provisional application No. 63/503,834, filed on May 23, 2023.

(51) Int. Cl. A61K 38/17; A61P 39/02

(52) U.S. Cl. CPC A61K 38/1703; A61P 39/02

(57) ABSTRACT

The present disclosure is directed to compounds and compositions for treating PSP such as, for example, therapeutically effective amount of a saxiphilin. This abstract is intended as a scanning tool for purposes of searching in the particular art and is not intended to be limiting of the present disclosure.

### CROSS REFERENCE TO RELATED APPLICATIONS

This application is a 35 U.S.C. § 371 national stage application of PCT/US2023/068244, which was filed Jun. 9, 2023, was entitled “Pharmaceutical Compositions and Methods of Treating PSP,” and claims priority to U.S. Provisional Patent Application No. 63/350,790, filed Jun. 9, 2022, and U.S. Provisional Patent Application No. 63/503,834, filed May 23, 2023, the contents of all of which are hereby incorporated by reference in their entireties.

### STATEMENT REGARDING FEDERALLY SPONSORED RESEARCH

This disclosure was made with government support under grant numbers HDTRA11910040 and HDTRA12110011 awarded by the United States Department of Defense. The United States government has certain rights in this invention.

### FIELD OF THE DISCLOSURE

The present disclosure is directed, in part, to saxiphilin proteins, nucleic acids encoding the same, pharmaceutical compositions comprising the same, kits comprising the same, and methods of treating PSP by administration of saxiphilins or functional fragments thereof. In some embodiments, the toxin is saxitoxin or derivatives of the same.

### BACKGROUND

Saxitoxin (STX), one of the most lethal non-peptidyl neurotoxins, blocks the bioelectrical signals in nerve and muscle required for life by inhibiting select voltage-gated sodium channel (Na_{V}) isoforms (Thottumkara et al., Hille, Llewellyn). The family of saxitoxins cause paralytic shellfish poisoning (PSP). Cyanobacteria and dinoflagellate species associated with oceanic red tides produce this bis-guanidinium small molecule and its congeners whose accumulation in seafood can cause PSP, a commercial fishing and public health hazard of growing importance due to climate change (Thottumkara et al., Llewellyn, Wiewse et al., Anderson et al.). Its extreme lethality has also earned STX the unusual distinction of being the only marine toxin declared a chemical weapon (Thottumkara et al., Llewellyn). Select vertebrates, particularly frogs, resist STX poisoning (Prinzmetal et al., Kao et al., Mahar et al., Abderemane-Ali et al.), a property that is thought to rely on the ability of the soluble ‘toxin sponge’ protein saxiphilin (Sxph) to sequester STX (Mahar et al., Abderemane-Ali et al.). There are no effective treatments known for these toxins.

### SUMMARY

In accordance with the purpose(s) of the disclosure, as embodied and broadly described herein, the disclosure, in some embodiments, relates to compounds and compositions useful in the treatment of PSP such as, for example, toxicity caused by STX or a structurally related family of toxins.

Thus, provided herein are methods for treating a PSP in a subject in need thereof, the method comprising administering to the subject an effective amount of compound selected from saxiphilin, or a pharmaceutically acceptable salt thereof.

Also provided are methods of neutralizing toxicity in a subject exposed to toxins disclosed herein, the method comprising administering to the subject an effective amount of a saxiphilin, or a pharmaceutically acceptable salt thereof.

Also provided are kits comprising a saxiphilin, and one or more selected from: (a) instructions for treating PSP; and (c) instructions for administering the compound in connection with treating PSP.

The present disclosure relates to pharmaceutical compositions and kits comprising: (i) a saxiphilin amino acid sequence, or analogs and functional fragments of a saxiphilin amino acid sequence; and (ii) a pharmaceutically acceptable carrier. In some embodiments, the saxiphilin is an amino acid sequence of the disclosure structurally related to an American Bullfrog saxiphilin sequence (SEQ ID NO:1), or functional fragments and variants thereof.

SEQ ID NO: 1:
MAPTFQTALFFTIISLSFAAPNAKQVRWCAISDLEQKKCNDLVGSCNVP
DITLVCVLRSSTEDCMTAIKDGQADAMFLDSGEVYEASKDPYNLKPIIA
EPYSSNRDLQKCLKERQQALAKKMIGHYIPQCDEKGNYQPQQCHGSTGH
CWCVNAMGEKISGTNTPPGQTRATCERHELPKCLKERQVALGGDEKVLG
RFVPQCDEKGNYEPQQFHGSTGYSWCVNAIGEEIAGTKTPPGKIPATCQ
KHDLVTTCHYAVAMVKKSSAFQFNQLKGKRSCHSGVSKTDGWKALVTVL
VEKKLLSWDGPAKESIQRAMSKFFSVSCIPGATQTNLCKQCKGEEGKNC
KNSHDEPYYGNYGAFRCLKEDMGDVAFLRSTALSDEHSEVYELLCPDNT
RKPLNKYKECNLGTVPAGTVVTRKISDKTEDINNFLMEAQKRQCKLFSS
AHGKDLMEDDSTLQLALLSSEVDAFLYLGVKLFHAMKALTGDAHLPSKN
KVRWCTINKLEKMKCDDWSAVSGGAIACTEASCPKGCVKQILKGEADAV
KLEVQYMYEALMCGLLPAVEEYHNKDDFGPCKTPGSPYTDFGTLRAVAL
VKKSNKDINWNNIKGKKSCHTGVGDIAGWVIPVSLIRRQNDNSDIDSFF
GESCAPGSDTKSNLCKLCIGDPKNSAANTKCSLSDKEAYYGNQGAFRCL
VEKGDVAFVPHTVVFENTDGKNPAVWAKNLKSEDFELLCLDGSRAPVSN
YKSCKLSGIPPPAIVTREESISDVVRIVANQQSLYGRKGFEKDMFQLFS
SNKGNNLLFNDNTQCLITFDRQPKDIMEDYFGKPYYTTVYGASRSAMSS
ELISACTIKHC

The present disclosure provides proteins or amino acid sequences comprising at least 80% sequence identity to SEQ ID NO:1 and comprising at least one of the following amino acid substitutions in SEQ ID NO: 1: an alanine for the isoleucine at position 782; an alanine for the tyrosine at position 558; an isoleucine for the tyrosine at position 558; an asparagine for the aspartic acid at position 785; an alanine for the lysine at position 789; an alanine for the threonine at position 563; a phenylalanine for a tyrosine at position 558; a glutamic acid for the glutamine at position 787; an alanine for a tyrosine at position 795; an alanine for the glutamine at position 787; a tyrosine for the phenylalanine at position 784; a phenylalanine for the isoleucine at position 782; an alanine for the phenylalanine at position 561; an alanine for the aspartic acid at position 785; a leucine for the phenylalanine at position 784; an alanine for the proline at position 727; an aspartic acid for the glutamic acid at position 540; an alanine for the phenylalanine at position 784; a glutamic acid for the aspartic acid at position 794; a cysteine for the phenylalanine at position 784; a asparagine for an aspartic acid at position 794; a serine for the phenylalanine at position 784; a glutamine for the glutamic acid at position 540; an alanine for the aspartic acid at position 794; an alanine for the glutamic acid at position 540.

The present disclosure provides proteins or amino acid sequences comprising at least 80% sequence identity to SEQ ID NO:1 and comprises a tyrosine for the isoleucine at position 559.

In some embodiments, the protein comprises at least 80% sequence identity to SEQ ID NO:1, and comprises at least one of the amino acid substitutions in SEQ ID NO:1. In some embodiments, the protein comprises at least 90% sequence identity to SEQ ID NO:1, and comprises at least one of the amino acid substitutions in SEQ ID NO:1. In some embodiments, the protein comprises at least 95% sequence identity to SEQ ID NO:1, and comprises at least one of the amino acid substitutions in SEQ ID NO:1. In some embodiments, the protein comprises at least 99% sequence identity to SEQ ID NO:1, and comprises at least one of the amino acid substitutions in SEQ ID NO:1. In some embodiments, the protein comprises at least 80% sequence identity to SEQ ID NO:2, and comprises at least one of the amino acid substitutions in SEQ ID NO:2. In some embodiments, the protein comprises at least 90% sequence identity to SEQ ID NO:2, and comprises at least one of the amino acid substitutions in SEQ ID NO:2. In some embodiments, the protein comprises at least 95% sequence identity to SEQ ID NO:2, and comprises at least one of the amino acid substitutions in SEQ ID NO:2. In some embodiments, the protein comprises at least 99% sequence identity to SEQ ID NO:2, and comprises at least one of the amino acid substitutions in SEQ ID NO:2.

In some embodiments, the protein comprises at least 80% sequence identity to SEQ ID NO:1, and comprises more than one of the amino acid substitutions in SEQ ID NO:1. In some embodiments, the protein comprises at least 90% sequence identity to SEQ ID NO: 1, and comprises more than one of the amino acid substitutions in SEQ ID NO: 1. In some embodiments, the protein comprises at least 95% sequence identity to SEQ ID NO: 1, and comprises more than one of the amino acid substitutions in SEQ ID NO:1. In some embodiments, the protein comprises at least 96% sequence identity to SEQ ID NO:1, and comprises more than one of the amino acid substitutions in SEQ ID NO:1. In some embodiments, the protein comprises at least 97% sequence identity to SEQ ID NO:1, and comprises more than one of the amino acid substitutions in SEQ ID NO:1. In some embodiments, the protein comprises at least 98% sequence identity to SEQ ID NO:1, and comprises more than one of the amino acid substitutions in SEQ ID NO:1. In some embodiments, the protein comprises at least 99% sequence identity to SEQ ID NO:1, and comprises more than one of the amino acid substitutions in SEQ ID NO:1.

In some embodiments, the protein or amino acid sequence comprises at least 80% sequence identity to SEQ ID NO:2, and comprises more than one of the amino acid substitutions in SEQ ID NO:2. In some embodiments, the protein comprises at least 90% sequence identity to SEQ ID NO:2, and comprises more than one of the amino acid substitutions in SEQ ID NO:2. In some embodiments, the protein comprises at least 95% sequence identity to SEQ ID NO:2, and comprises more than one of the amino acid substitutions in SEQ ID NO:2. In some embodiments, the protein comprises at least 96% sequence identity to SEQ ID NO:2, and comprises more than one of the amino acid substitutions in SEQ ID NO:2. In some embodiments, the protein comprises at least 97% sequence identity to SEQ ID NO:2, and comprises more than one of the amino acid substitutions in SEQ ID NO:2. In some embodiments, the protein comprises at least 98% sequence identity to SEQ ID NO:2, and comprises more than one of the amino acid substitutions in SEQ ID NO:2. In some embodiments, the protein comprises at least 99% sequence identity to SEQ ID NO:2, and comprises more than one of the amino acid substitutions in SEQ ID NO:2.

In some embodiments, the protein or amino acid sequence comprises SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15. In some embodiments, the protein or amino acid sequence comprises at least about 70% or about 80% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, or SEQ ID NO:15 but is free of SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15, respectively.

The present invention also provides nucleic acids encoding any of the proteins described above. In some embodiments, the nucleic acid comprises. The present invention also provides vectors comprising any of the nucleic acids described above encoding any of the proteins described above. In some embodiments, the vector is a plasmid. In some embodiments, the vector is a retrovirus.

In some embodiments, the protein comprises at least 80% sequence identity to SEQ ID NO:1, and comprises more than one of the amino acid substitutions in SEQ ID NO:1. In some embodiments, the mutation is a substitution or deletion mutation 540, 558; 561, 563, 727, 782, 784, 785, 787, 789, 794 and/or 795.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTIONS OF THE DRAWINGS

FIG. 1. Alanine scan of RcSxph binding.

FIGS. 1A, and 1B. Exemplar thermofluor (TF) assay results for RcSxph (FIG. 1A) in the presence of the indicated concentrations of STX (left) and TTX (right) and Select RcSxph mutants (FIG. 1B) in the presence of STX. STX and TTX concentrations are 0 nM (black), 19.5 nM (blue), 625 nM (cyan), 5000 nM (orange), and 20000 nM (red). Dashed lines indicate ΔTm. FIG. 1C. F-STX diagram. STX and fluorescein (F) moieties are highlighted blue and yellow, respectively. FIG. 1D. Exemplar Fluorescence polarization (FP) binding curves and Kds for RcSxph and the indicated mutants. FIG. 1E. Comparison of RcSxph mutant ΔTm and ΔΔG values (line y=3.49−0.7523x, R²=0.886). FIG. 1F. Exemplar isotherms for titration of 100 μM STX into 10 μM RcSxph. 100 μM STX into 10 μM RcSxph F561A, 100 μM STX into 10 μM RcSxph E540D, and 300 μM STX into 30 μM RcSxph D794E. Kd and ΔH values are indicated. FIG. 1G. Comparison of ΔG_{ITC} for STX and ΔG_{FP} for F-STX for RcSxph and mutants. Purple box highlights region of good correlation. Orange box indicates region outside of the ITC dynamic range. (line shows x=y)

FIGS. 2A through 2D. Energetic fingerprint of STX recognition by RcSxph.

FIG. 2A. ΔΔG comparisons for the indicated RcSxph STX binding pocket mutants relative to wild-type RcSxph. Colors indicate: ΔΔG<−1 kcal mol⁻¹ (blue), −1 ΔΔG≤0 kcal mol⁻¹ (light blue), 0≥ΔΔG≥1 kcal mol⁻¹ (yellow), 1≥ΔΔG≥2 kcal mol⁻¹ (orange), 2≥ΔΔG≥3 kcal mol⁻¹ (red orange), and ΔΔG≥3 (red). FIG. 2B. Energetic map of alanine scan mutations on STX binding to the RcSxph STX binding pocket (PDB:6O0F) (Yen et al.). Second shell sites are in italics. Colors are as in ‘A’. C and D, Structural interactions of STX with C, RcSxph (PDB:6O0F) (Yen et al.) and D, human Na_{V}1.7 (PDB:6J8G) (Shen et al. 2019). Residues that are energetically important for the STX interaction are shown in space filling. Na_{V}1.7 selectivity filter ‘DEKA’ ring residues are shown (white). Italics indicate corresponding residue numbers for rat Na_{V}1.4 (Thomas-Tran and Du Bois).

FIG. 3. Structures of enhanced affinity RcSxph mutants.

FIG. 3A. Superposition of the STX binding pockets of RcSxph-Y558A (purple) and the RcSxph-Y558A:STX complex (light blue). FIG. 3B. Superposition of the STX binding pockets of RcSxph-Y558I (pale yellow) and the RcSxph-Y558I:STX complex (splitpea).

FIG. 3C. Superposition of the STX binding pockets of STX bound complexes of RcSxph (PDB: 6O0F) (Yen et al.), RcSxph-Y558A (purple), and RcSxph-Y558I (splitpea). STX from the RcSxph is firebrick.

FIG. 4. RcSxph mutants have differential effects on PtNa_{V}1.4 rescue from STX block.

FIG. 4A-E, Exemplar two-electrode voltage clamp recordings of PtNa_{V}1.4 expressed in Xenopus oocytes in the presence of 100 nM STX and indicated [Sxph]:[STX] ratios for RcSxph (FIG. 4A), RcSxph E540A (FIG. 4B), RcSxph Y558I (FIG. 4C), RcSxph P727A (FIG. 4D), and RcSxph I782A (FIG. 4E). Inset shows the stimulation protocol. FIG. 4F. [Sxph]:[STX] dose response curves for RcSxph (black, open circles), RcSxph E540A (inverted triangles), RcSxph Y558I (triangles), RcSxph P727A (diamonds), and RcSxph I782A (squares) in the presence of 100 nM STX. Lines show fit to the Hill equation.

FIG. 5. Sxph family member properties.

FIG. 5A. Comparison of the human transferrin (TF) Fe³⁺ ligand positions (UniProtKB: P02787), RcSxph STX binding motif residues (Yen et al.), and number of Thy1 domains for Sxphs from R. catesbaiana (PDB:600D) (Yen et al.), N. parkeri (NCBI: XP_018410833.1) (Yen et al.), M. aurantiaca, D. tinctorius, O. sylvatica, R. imitator, P. terribilis, E. tricolor, A. femoralis, R. marinus, B. bufo (NCBI:XM_040427746.1), and B. garagarizans. (NCBI:XP_044148290.1). TF Fe³⁺ (orange) and carbonate (blue) ligands are indicated. Blue highlights indicate residue conservation. FIG. 5B. Comparison of STX binding pocket for the indicated Sxphs. Numbers denote RcSxph positions. Conserved residues are highlighted. Asterix indicates second shell sites. FIG. 5C. Exemplar TF curves for NpSxph, RiSxph, OsSxph, and MaSxph in the presence of the indicated concentrations of STX (purple box) or TTX. (green box) ΔTm values are indicated. FIG. 5D. Exemplar FP binding curves and Kds for NpSxph (green), RiSxph (blue), OsSxph (orange), and MaSxph (purple). FIG. 5E. Exemplar NpSxph I559Y TF curves in the presence of the indicated concentrations of STX (purple box) or TTX (green box) and FP binding (green). ΔTm and Kd values are indicated. Error bars are S.E.M.

FIG. 6. NpSxph and NpSxph:STX structures.

FIG. 6A. Cartoon diagram of the NpSxph:STX complex. N1 (light green), N2 (green), Thy1-1 (light orange), Thy1-2 (orange), C1 (marine), and C2 (light blue) domains are indicated. STX (pink) is shown in space filling representation. FIG. 6B. Comparison of STX binding pocket for apo-NpSxph (yellow) and NpSxph:STX (marine). STX (pink) is shown as ball and stick. FIG. 6C. Comparison of NpSxph (marine) and RcSxph (orange) (PDB:6O0F) (Yen et al.) STX binding sites. STX from NpSxph and RcSxph complexes is pink and orange, respectively. FIG. 6D. Comparison of NpSxph (marine) and RcSxph-Y558I (splitpea) STX binding sites. STX from NpSxph and RcSxph-Y558I complexes is pink and splitpea, respectively. RcSxph and RcSxph-Y558I residue numbers in FIG. 6C and FIG. 6D are indicated in italics.

FIG. 7. RcSxph thermofluor (TF) assay.

FIG. 7A. Exemplar thermofluor (TF) assay results for RcSxph in the presence of the indicated concentrations of STX. Curves for RcSxph, E540A, P727A, Y558A, F561A, and T563A are identical to those shown in FIGS. 1A and 1B. DTm values are indicated. FIG. 7B. Baseline Tm values for RcSxph and the indicated mutants. FIG. 7C. Plot of Tm vs. DTm for the proteins in FIG. 7B. Error bars are S.E.M.

FIG. 8. F-STX NMR spectrum.

FIG. 8. ¹H NMR (600 MHz, D₂O) δ 8.05 (d, J=8.1 Hz, 1H), 7.94 (d, J=8.9 Hz, 1H), 7.48 (s, 1H), 6.95 (d, J=9.0 Hz, 2H), 6.79 (s, 2H), 6.67 (dt, J=9.1, 2.2 Hz, 2H), 4.60 (d, J=1.2 Hz, 1H), 4.09-4.05 (m, 1H), 3.89 (dd, J=11.6, 5.2 Hz, 1H), 3.70 (dt, J=10.1, 5.5 Hz, 1H), 3.64 (dd, J=8.7, 5.4 Hz, 1H), 3.47-3.42 (m, 1H), 3.27 (t, J=6.6 Hz, 2H), 2.97-2.89 (m, 2H), 2.36-2.33 (m 1H), 2.30-2.24 (m, 1H), 1.48-1.45 (m, 2H), 1.32-1.29 (m, 2H), 1.25-1.21 (m, 4H) ppm

FIG. 9. Structure of the RcSxph:F-STX: complex.

FIG. 9A. Exemplar electron density (Is) for RcSxph (dark gray) and F-STX (light gray). FIG. 9B. RcSxph:F-STX: B-factors for the F-STX ligand and select binding site residues. FIG. 9C. Superposition of the STX binding sites of the RcSxph:F-STX; and RcSxph:STX (PDB:6O0F) (Yen et al.) complexes.

FIG. 10. RcSxph fluorescence polarization (FP) assay.

FIG. 10. Exemplar FP binding curves and Kds for RcSxph and the indicated mutants. Curves for RcSxph, E540A, P727A, Y558A, F561A, and T563A are identical to those shown in FIG. 1D. Colored boxes and lines in correspond to DDG classifications in Table 1. Error bars are S.E.M.

FIG. 11. RcSxph and NpSxph Isothermal titration calorimetry.

FIG. 11. Exemplar ITC isotherms for 100 μM STX into 10 μM RcSxph Y558A (FIG. 11A), 100 μM STX into 10 μM RcSxph Y558I (FIG. 11B), 100 μM STX into 10 μM RcSxph P727A (FIG. 11C), 100 μM STX into 9.7 μM NpSxph (FIG. 11D), and 100 μM STX into 7.9 μM NpSxph I559Y (FIG. 11E). FIG. 11F. Comparison of DG_{ITC} for STX and DG_{FP} for F-STX for RcSxph, NpSxph, and indicated mutants. Purple box highlights region of good correlation. Orange box indicates region outside of the ITC dynamic range. RcSxph data are identical to FIG. 1G.

FIG. 12. RcSxph Y558A and RcSxph-Y558I structures and STX complexes.

FIG. 12. Exemplar electron density (1.5 s) for RcSxph Y558A (purple)(FIG. 12A), RcSxph-Y558A:STX (light blue)(FIG. 12B), RcSxph-Y558I (pale yellow)(FIG. 12C), and RcSxph-Y558I:STX (splitpea)(FIG. 12D). Select residues and STX are indicated.

FIG. 13. Frog Sxph sequence alignment.

FIG. 13. Sxph sequence alignment for RcSxph, NpSxph, MaSxph, DtSxph, OsSxph, RiSxph, PtSxph, EtSxph, and AfSxph. Domains and secondary structure are from RcSxph. N1 (dark green), N2 (light green), Thy1 domains (orange), C1 (marine), C2 (cyan). STX binding site residues are indicated by stars and colored based on the alanine scan results in Table 1. Residues corresponding to transferrin Fe³⁺ and carbonate ligands are indicated by orange and blue hexagons, respectively and highlighted (Yen et al., Lambert et al.).

FIG. 14. Toad Sxph sequence alignment.

Sxph sequence alignment for RcSxph, and toad saxiphilins RmSxph, BbSxph (NCBI:XM_040427746.1), and BgSxph(NCBI:XP_044148290.1). Domains and secondary structure are from RcSxph. N1 (dark green), N2 (light green), Thy1 domains (orange), C1 (marine), C2 (cyan). STX binding site residues are indicated by stars and colored based on the alanine scan results in Table 1. Residues corresponding to transferrin Fe³⁺ and carbonate ligands (Yen et al., Lambert et al.) are indicated by orange and blue hexagons, respectively and highlighted. Only beginning and ends of the Thy1 domains are shown. Total number of Thy1 domains are indicated.

FIG. 15. Thy1 domain sequence alignment.

Thy1 domains from RcSxph, NpSxph, MaSxph, DtSxph, OsSxph, RiSxph, PtSxph, EtSxph, AfSxph, RmSxph, BbSxph, and BgSxph and the type (1A or 1B) are shown. Secondary structure from RcSxph Thy1-1 is shown. Cysteine are highlighted. SS4-SS8 indicate disulfide numbers from RcSxph. Loop regions are indicated.

FIG. 16. NpSxph structure and comparisons with RcSxph.

FIGS. 16A and 16B. Exemplar electron density for NpSxph (2Fo-Fc, 1.5 s, grey) and (Fo-Fc, 3.0 s, green) (FIG. 16A), NpSxph:STX (2Fo-Fc, 1.5 s, grey). NpSxph (marine), STX (pink), and PEG400 (yellow) (FIG. 16B) are shown. STX (pink) from the NpSxph:STX complex is shown in FIG. 16A to compare with the PEG400 position. Select residues are labelled. FIG. 16C. NpSxph and RcSxph superposition using the C-lobes. N- and C-lobes are green/light green and marine/light blue for NpSxph and RcSxph, respectively. Arrow indicate relationships between NpSxph and RcSxph N-lobes. FIG. 16D. Superposition of NpSxph (green) and RcSxph (light green) N-lobes. FIG. 16E. Superposition of NpSxph (marine) and RcSxph (light blue) C-lobes. FIG. 16F. Cartoon diagram of NpSxph and RcSxph superposition from FIG. 16C showing the change in Thy1 domain positions. NpSxph Thy1 domains (orange) and RcSxph Thy1 domains (magenta) are indicated. FIG. 16G. Cartoon diagram of NpSxph and RcSxph Thy1 domains superposed on Thy1-1. NpSxph and RcSxph Thy1-1 and Thy1-2 are light orange and pink and orange and magenta, respectively. FIG. 16H. Superposition of individual NpSxph and RcSxph Thy1-1 and Thy1-2 domains. Disulfide bonds are indicated.

FIG. 17. Structure of the NpSxph:F-STX complex.

FIG. 17A. Exemplar electron density for NpSxph:F-STX(2Fo-Fc, 1.5 s, grey). NpSxph (cyan) and F-STX (orange). FIG. 17B. Comparison of NpSxph:STX (marine) and NpSxph:F-STX STX binding sites. STX from NpSxph is pink. F-STX is orange. Select residues are indicated.

FIG. 18. In vivo rescue. FIG. 18 depicts a summary of IP injection experiments testing the effects of RcSxph. Dots indicate mice having no symptoms. X's indicate mice showing symptoms of STX or TTX poisoning. Box highlights the key rescue experiment that provides the first evidence that RcSxph can act as an STX countermeasure.

### DETAILED DESCRIPTION

The present invention can be understood more readily by reference to the following detailed description of the disclosure and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc.

The term “or” as used herein shall only be interpreted as indicating exclusive alternatives (i.e., “one or the other but not both”) when preceded by terms of exclusivity, “either,” “one of,”. “only one of,” or “exactly one of.”

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein, the terms “biologically significant” refers to an amount or concentration of enzymatic reaction product or enzyme in a sample whose quantity of binding that is detected and is statistically significant as compared to a control when the amount or concentration is normalized for a control. In some embodiments, the terms is used to describe the amount of toxin that is present in a sample at a level sufficient to cause a dysfunctional biological effect. In some embodiments, the biologically significant amount of amino acid sequence (or saxiphilin) is the amount sufficient to bind or neutralize toxins disclosed herein. In some embodiments, the biologically significant amount of amino acid sequence disclosed herein is the amount sufficient to characterize a sample as toxic. In some embodiments, the biologically significant amount of toxin is the amount sufficient to cause PSP. In some embodiments, the biologically significant amount of toxin is the amount sufficient to characterize a sample as being toxic.

As used herein, the term “kit” refers to a set of components provided in the context of a system for delivering materials or diagnosing a subject with having been contaminated with a disclosed toxin or exposed to a disclosed toxin. Such delivery systems may include, for example, systems that allow for storage, transport, or delivery of various diagnostic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a diagnostic assay comprising two or more separate containers that each contain a subportion of total kit components. Containers may be delivered to an intended recipient together or separately. For example, a first container may contain a petri dish or polystyrene plate for use in a cell culture assay, while a second container may contain cells, such as control cells. As another example, the kit may comprise a first container comprising a solid support such as a chip or slide with one or a plurality of ligands with affinities to one or a plurality of biomarkers disclosed herein and a second container comprising any one or plurality of reagents necessary for the detection and/or quantification of the amount of biomarkers in a sample. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits.

As used herein, the phrase “integer from about X to about Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, “cell culture” means growth, maintenance, transfection, or propagation of cells, tissues, or their products. As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo. In some embodiments, culture medium means solution capable of sustaining the growth of the targeted cells either in vitro.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the term “mammal” means any animal in the class Mammalia such as rodent (i.e., a mouse, a rat, or a guinea pig), a monkey, a cat, a dog, a cow, a horse, a pig, or a human. In some embodiments, the mammal is a human. In some embodiments, the mammal refers to any non-human mammal. The present disclosure relates to any of the methods or compositions of matter disclosed herein wherein the sample is taken from a mammal or non-human mammal. The present disclosure relates to any of the methods or compositions of matter disclosed herein wherein the sample is taken from a human or non-human primate.

In some embodiments, a “toxin” is a naturally occurring non-peptidyl neurotoxin in the family of saxitoxin. In some embodiments, a “toxin” is a neurotoxin. In some embodiments, a “toxin” is saxitoxin. In some embodiments, a “toxin” is a saxitoxin family member. There are 57 known saxitoxin family members (Wiese et al., 2010 March Drugs 8:2185-2211, which is hereby incorporated by reference in its entirety). A toxin may comprise one or more than one saxitoxin family member.

In some embodiments, a toxin is a compound having a structure represented by a formula:

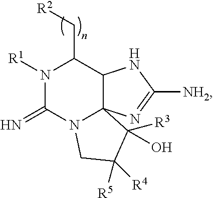

wherein n is selected from 0, 1, and 2; wherein each of R¹ and R³ is independently selected from hydrogen and —OH; wherein R² is selected from hydrogen, —OH, and —OC(O)R¹⁰; wherein R¹⁰, when present, is selected from —NH₂, —CH₃, —NHSO₂H, —NHSO₃⁻, —NHSO₃H, and Ar¹; wherein Ar¹, when present, is a C6 aryl substituted with 0, 1, 2, or 3 groups independently selected from —OH, —SO₃⁻, and —SO₃H; and wherein each of R⁴ and R⁵ is independently selected from hydrogen, —OH, —OSO₃H, and —OSO₃⁻, or a salt thereof.

In some embodiments, a toxin is a structure represented by a formula selected from:

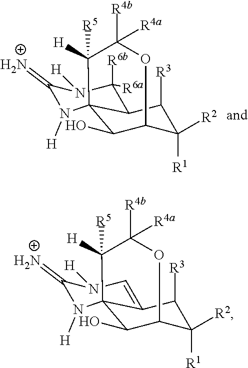

wherein R¹ is selected from hydrogen and —OH; wherein R² is selected from —CH₃, —CH₂OH, and —CH(OH)₂; wherein R³ is hydrogen and wherein each of R^{4a} and R^{4b} together comprise ═O, or wherein R³ and R^{4a} together comprise —O— and wherein R^{4b} is —OH; and wherein R⁵ is —OH and wherein each of R^{6a} and R^{6b} is independently selected from hydrogen and —OH, or wherein R⁵ and R^{6b} together comprise —O— and wherein R^{6a} is hydrogen; or a salt thereof.

In some embodiments, the disclosure relates to a method of treating a subject exposed to a toxin. In some embodiments, the disclosure relates to a method of detecting a toxin in a sample from a human. In some embodiments, the disclosure relates to a method of detecting a toxin in a sample from a mollusk.

TABLE 5
Saxitoxin structures

| | | |
|---|---|---|
| Saxitoxin | STX | 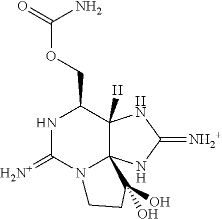 |
| Decarbamoyl Saxitoxin | dcSTX | 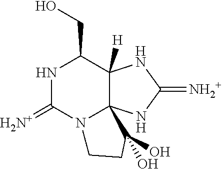 |
| Neosaxitoxin | neoSTX | 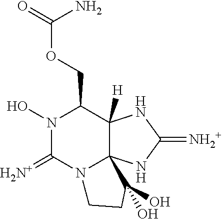 |
| Decarbamoyl Neosaxitoxin | dc-neoSTX | 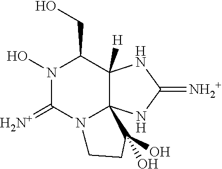 |
| Gonyautoxin IV | GTX5 (B1) | 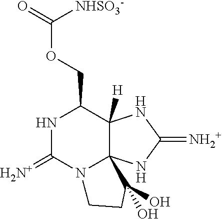 |
| | GTX6 | 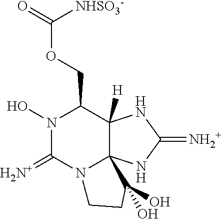 |
| | GTX2/3 | 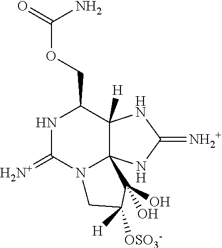 |
| | | 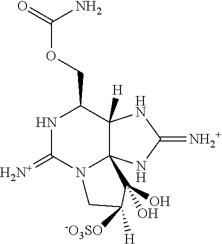 |
| | dcGTX2/3 | 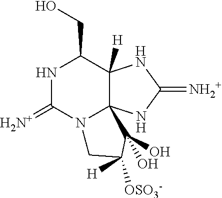 |
| | | 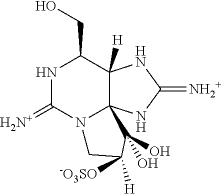 |
| | C1/C2 | 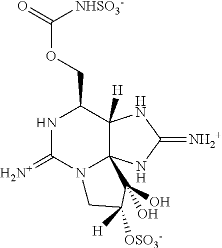 |
| | | 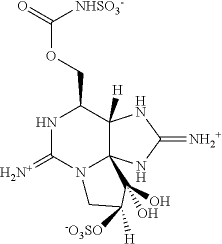 |
| | GTX1/4 | 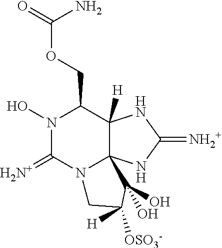 |
| | | 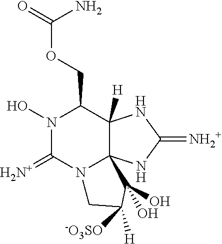 |
| | TTX | 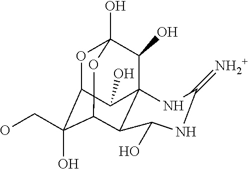 [SMILES: [NH2+]=C1NC(O)C2C3OC4(O)OC([C@@H](O)C2(N1)[C@@H]4O)C3(O)CO] |
| | C13-OBz | 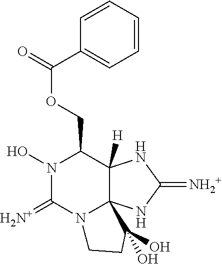 |
| | C13-OAc | 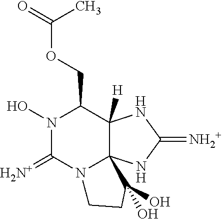 |
| | C13—O-2- CH3-Ph | 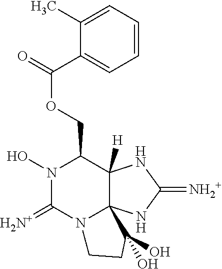 |
| | C13—O-2- F—Ph | 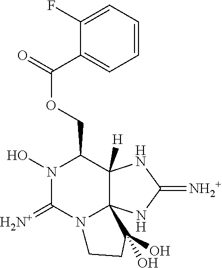 |
| | C13—O-3- CH3—Ph | 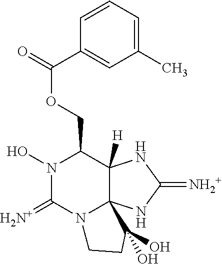 |
| | C13—O-3- F—Ph | 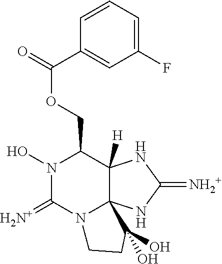 |
| | C13—O-4- CH3—Ph | 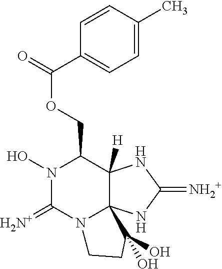 |
| | C13—O-4- F—Ph | 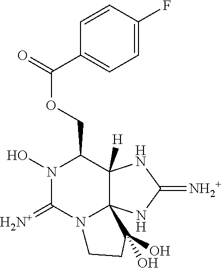 |
| | C13—O-4- OH—Ph | 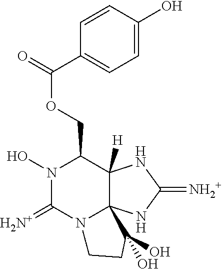 |
| | C13—O-3,4- (OH)₂—Ph | 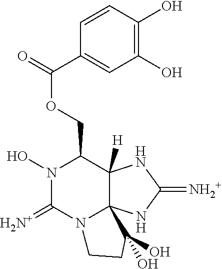 |

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

As used herein, “specific for” or “specifically binds to” means that the binding affinity of a substrate to a specified target nucleic acid sequence, such as a tripeptidyl peptidase, is statistically higher than the binding affinity of the same substrate to a generally comparable, but non-target amino acid sequence. The substrate's Kd to each nucleotide sequence can be compared to assess the binding specificity of the substrate to a particular target nucleotide sequence.

Human or non-human variants of the enzymes above are contemplated by the methods, systems, and devices disclosed herein. Variants of these enzymes include sequences that are at least 70% homologous or identical to the human sequences above. As used herein, the term “variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule or amino acid sequence of the disclosure will have at least about 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular nucleic acid molecule of the disclosure (i.e., the reference amino acid sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 87%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 20, 15, 10, 9, 8, 7, 6, 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X, and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder associated toxicity caused by saxitoxin such as, for example, a PSP, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition.

The term “contacting” or “exposing” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In some embodiments, an IC₅₀ can refer to the concentration of a substance that is required for 50% inhibition in vivo, as further defined elsewhere herein.

As used herein, “EC₅₀” is intended to refer to the concentration of a substance (e.g., a compound or a drug) that is results in a half-maximal response (i.e., 50% of the maximum response) of a biological process, or component of a process, including a protein, subunit, organelle, ribonucleoprotein, etc. In some embodiments, an EC₅₀ can refer to the concentration of a substance that is required to achieve 50% of the maximum response in vivo, as further defined elsewhere herein.

The saxiphilins according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

“Effective amount” refers to an amount of a compound, material, or composition, as described herein effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art. The effective amount of the composition may be dependent on any number of variables, including without limitation, the species, breed, size, height, weight, age, overall health of the subject, the type of formulation, the mode or manner or administration, the type and/or severity of the particular condition being treated, or the need to modulate the activity of the molecular pathway induced by association of the analog to its receptor. The appropriate effective amount can be routinely determined by those of skill in the art using routine optimization techniques and the skilled and informed judgment of the practitioner and other factors evident to those skilled in the art. A therapeutically effective dose of the saxiphilins described herein may provide partial or complete biological activity as compared to the biological activity induced by the wild-type or naturally occurring polypeptides upon which the saxiphilins are derived. A therapeutically effective dose of the proteins or amino acids described herein may provide a sustained biochemical or biological affect and/or an increased resistance to toxicity when administered to a subject as compared with the normal affect observed when the naturally occurring and fully processed translated protein is administered to the same subject.

The term “fragment” refers to any analog of a naturally occurring polypeptide disclosed herein that comprises at least 4 amino acids identical to the naturally occurring polypeptide upon which the analog is based. The term “functional fragment” refers to any fragment of saxiphilin disclosed herein that comprises at least about 75% sequence identity to any of those amino acid sequence chosen from SEQ ID NO: 1 through 15, and shares the function of the naturally occurring polypeptide upon which the saxiphilin is based. In some embodiments, the functional nature of the fragment is to bind or associate a disclosed toxin, and, in some embodiments, neutralize the toxin if within a subject.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad embodiment, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in some embodiments, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl,” as used herein, refers to a monovalent saturated, straight- or branched-chain hydrocarbon radical, having unless otherwise specified, 1-6 carbon atoms. Examples of alkyl radicals include, but are not limited to, methyl, ethyl, n-propyl, isopropyl, n-butyl, iso-butyl, sec-butyl, n-pentyl, tert-pentyl, neopentyl, sec-pentyl, 3-pentyl, sec-isopentyl, hexyl, 2-methylpentane, 3-methylpentane, 2,2-dimethylbutane, 2,3-dimentybutane and the like. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “heteroalkyl,” as used herein, refers to an alkyl group containing at least one heteroatom. Suitable heteroatoms include, but are not limited to, O, N, Si, P, and S, wherein the nitrogen, phosphorous and sulfur atoms are optionally oxidized, and the nitrogen heteroatom is optionally quaternized. Heteroalkyls can be substituted as defined above for alkyl groups.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “9- to 10-membered carbocyclyl” means a 9- or 10-membered monocyclic, bicyclic (e.g., a bridged or spiro bicyclic ring), polycyclic (e.g., tricyclic), or fused hydrocarbon ring system that is saturated or partially unsaturated. The term “9- to 10-membered carbocyclyl” also includes saturated or partially unsaturated hydrocarbon rings that are fused to one or more aromatic or partially saturated hydrocarbon rings (e.g., dihydroindenyl and tetrahydronaphthalenyl). Bridged bicyclic cycloalkyl groups include, without limitation, bicyclo[4.3.1]decanyl and the like. Spiro bicyclic cycloalkyl groups include, e.g., spiro[3.6]decanyl, spiro[4.5]decanyl, spiro [4.4]nonyl and the like. Fused cycloalkyl rings include, e.g., decahydronaphthalenyl, dihydroindenyl, decahydroazulenyl, octahydroazulenyl, tetrahydronaphthalenyl, and the like. It will be understood that when specified, optional substituents on a carbocyclyl (e.g., in the case of an optionally substituted cycloalkyl) may be present on any substitutable position and, include, e.g., the position at which the carbocyclyl group is attached.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, cycloheptyl, cyclooctyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein. In various aspects, the cycloalkyl group and heterocycloalkyl group can be monocyclic, bicyclic (e.g., bridged such as, for example, bicyclo[4.3.1]decanyl or spiro such as, for example, spiro[3.6]decanyl, spiro[4.5]decanyl, spiro [4.4]nonyl), polycyclic (e.g., tricyclic), or a fused hydrocarbon ring system that is saturated or partially unsaturated (e.g., decahydronaphthalenyl, dihydroindenyl, decahydroazulenyl, octahydroazulenyl, tetrahydronaphthalenyl).

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “heterocycle” or “heterocyclyl” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” The heterocycle can be monocyclic, bicyclic (e.g., spiro or bridged), polycyclic, or a fused system that is saturated or partially saturated. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2, or 3 ring heteroatoms or wherein a pyridine ring is fused to a 5- or a 6-membered ring containing 1, 2, or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “9-membered fused heterocyclyl” means a 9-membered saturated or partially unsaturated fused monocyclic heterocyclic ring comprising at least one oxygen heteroatom and optionally two to four additional heteroatoms independently selected from N, O, and S. The terms “heterocycle,” “heterocyclyl,” “heterocyclyl ring,” “heterocyclic group,” “heterocyclic moiety,” and “heterocyclic radical,” are used interchangeably herein. A heterocyclyl ring can be attached to its pendant group at any heteroatom or carbon atom that results in a stable structure. Examples of fused saturated or partially unsaturated heterocyclic radicals comprising at least one oxygen atom include, without limitation, dihydrobenzofuranyl, dihydrofuropyridinyl, octahydrobenzofuranyl, and the like. Where specified as being optionally substituted, substituents on a heterocyclyl (e.g., in the case of an optionally substituted heterocyclyl) may be present on any substitutable position and include, e.g., the position at which the heterocyclyl group is attached.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “heteroaryl,” as used herein, refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “5- or 6-membered heteroaryl” refers to a 5- or 6-membered aromatic radical containing 1-4 heteroatoms selected from N, O, and S. Nonlimiting examples include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. When specified, optional substituents on a heteroaryl group may be present on any substitutable position and, include, e.g., the position at which the heteroaryl is attached.

The term “aldehyde” as used herein is represented by the formula C(O)H. Throughout this specification “C(O)” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain some embodiments, unless expressly indicated to the contrary, individual substituents can be further optionally substituted (i.e., further substituted or unsubstituted).

In some embodiments, a structure of a compound can be represented by a formula:

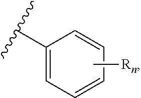

which is understood to be equivalent to a formula:

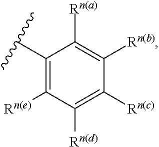

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further embodiments, a structure of a compound can be represented by a formula:

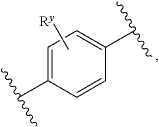

wherein R^{y} represents, for example, 0-2 independent substituents selected from A¹, A², and A³, which is understood to be equivalent to the groups of formulae:
wherein R^{y} represents 0 independent substituents

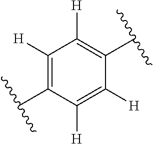

wherein R^{y} represents 1 independent substituent

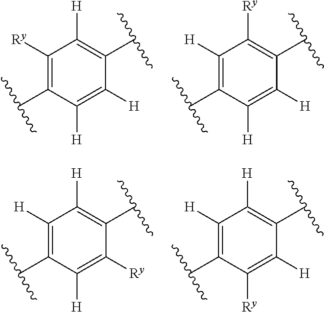

wherein R^{y} represents 2 independent substituents

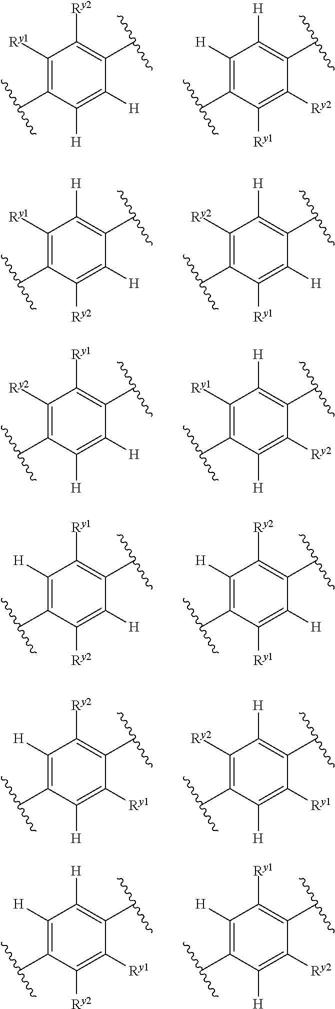

Again, by “independent substituents,” it is meant that each R substituent can be independently defined. For example, if in one instance R^{y1} is A¹, then R^{y2} is not necessarily A¹ in that instance.

In some further embodiments, a structure of a compound can be represented by a formula,

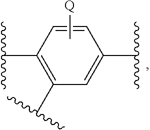

wherein, for example, Q comprises three substituents independently selected from hydrogen and A, which is understood to be equivalent to a formula:

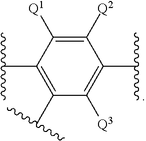

Again, by “independent substituents,” it is meant that each Q substituent is independently defined as hydrogen or A, which is understood to be equivalent to the groups of formulae:
wherein Q comprises three substituents independently selected from H and A

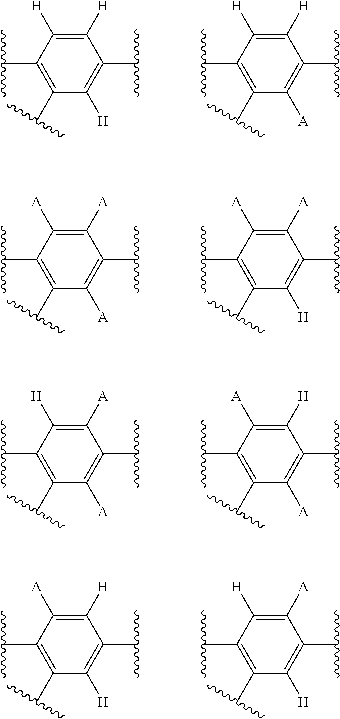

In some embodiment, the disclosed compounds exists as geometric isomers. “Geometric isomer” refers to isomers that differ in the orientation of substituent atoms in relationship to a cycloalkyl ring, i.e., cis or trans isomers. When a disclosed compound is named or depicted by structure without indicating a particular cis or trans geometric isomer form, it is to be understood that the name or structure encompasses one geometric isomer free of other geometric isomers, mixtures of geometric isomers, or mixtures enriched in one geometric isomer relative to its corresponding geometric isomer. When a particular geometric isomer is depicted, i.e., cis or trans, the depicted isomer is at least about 60%, 70%, 80%, 90%, 99%, or 99.9% by weight pure relative to the other geometric isomer.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The pharmaceutical composition can comprise any pharmaceutically acceptable carrier or ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

Disease, disorder, and condition are used interchangeably herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

In some embodiments, the term “effective amount” or “therapeutically effective amount” refers to an amount that is sufficient to achieve the desired result (e.g., that will elicit a biological or medical response of a subject e.g., a dosage from about 0.01 to about 100 mg/kg body weight/day) or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various embodiments, a preparation can be administered in a “prophylactically effective amount”; that is, an amount effective for prevention of a disease or condition.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. The term “salt” refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the compound in question. Salts include those formed from hydrochloric, hydrobromic, sulphuric, phosphoric, citric, tartaric, lactic, pyruvic, acetic, succinic, fumaric, maleic, methanesulphonic and benzenesulphonic acids.

In some embodiments, salts of the compositions comprising a saxiphilin or functional fragment thereof may be formed by reacting the free base, or a salt, enantiomer or racemate thereof, with one or more equivalents of the appropriate acid. In some embodiments, pharmaceutical acceptable salts of the present invention refer to analogs having at least one basic group or at least one basic radical. In some embodiments, pharmaceutical acceptable salts of the present invention comprise a free amino group, a free guanidino group, a pyrazinyl radical, or a pyridyl radical that forms acid addition salts. In some embodiments, the pharmaceutical acceptable salts of the present invention refer to analogs that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present invention may be found in their anhydrous form or as in hydrated crystalline form (i.e., complexed or crystallized with one or more molecules of water).

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). In some embodiments, the subject is a human or human in need of treatment for PSP or neurotoxicity.

The term “associated” or “associated with” in the context of a substance or substance activity or function associated with a disease (e.g., a protein associated disease, a symptom associated with a PSP, a symptom associated with NO neuron activity) means that the disease (e.g., the PSP) is caused by (in whole or in part), or a symptom of the disease is caused by (in whole or in part) the substance or substance activity or function. For example, a symptom of a gut motility disease or condition may be a symptom that results (entirely or partially) from modulation of NO neuron activity (e.g., induction of colonic motility). As used herein, what is described as being associated with a disease, if a causative agent, could be a target for treatment of the disease. For example, PSP, may be treated with an agent (e.g., compound as described herein) effective for modulating neurotoxicity of a toxin.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be an amino acid sequence disclosed herein and a toxin disclosed herein. In some embodiments contacting includes allowing a saxiphilin described herein to interact with a toxin and neutralize or inhibit its biological effect or effects.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The symbol “ [symbol] ” denotes the point of attachment of a chemical moiety to the remainder of a molecule or chemical formula.

As defined herein, the term “activation,” “activate,” “activating,” and the like in reference to a protein-activator (e.g., agonist) interaction means positively affecting (e.g., increasing) the activity or function of the protein relative to the activity or function of the protein in the absence of the activator. In some embodiments, activation refers to an increase in the activity of a signal transduction pathway or signaling pathway. Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein that may modulate the level of another protein or increase/decrease cell survival.

The term “modulator” refers to a composition that increases or decreases the level of a target molecule or the function of a target molecule. In some embodiments, the modulator is a modulator of neurotoxicity. In some embodiments, the modulator is a modulator of neurotoxicity and is a compound that reduces the severity of one or more symptoms of a disease associated with neurotoxicity, such as PSP. In some embodiments, a modulator is a compound that reduces the severity of one or more symptoms of a PSP caused by exposure to or contamination by a saxitoxin.

“Patient” or “subject in need thereof” can be used interchangeably and refers to a living organism suffering from or prone to a disease or condition that can be treated by administration of a compound or pharmaceutical composition, as provided herein. Non-limiting examples include humans, other mammals, bovines, cats, rats, mice, dogs, monkeys, goat, sheep, cows, horses, pigs, deer, and other non-mammalian animals. In some embodiments, a patient is human.

“Disease” or “condition” refer to a state of being or health status of a patient or subject capable of being treated with a compound, pharmaceutical composition, or method provided herein. In some embodiments, the disease is a disease related to (e.g., characterized by) modulation of neurotoxicity. In some embodiments, the disease is a PSP.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs.

The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples of saxiphilins and functional fragments thereof) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms. Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of a PSP), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating symptoms of PSP as further described herein, or with adjunctive agents that may not be effective alone, but may contribute to the efficacy of the active agent.

In some embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In some embodiments, the active and/or adjunctive agents may be linked or conjugated to one another. In some embodiments, the compounds described herein may be combined with treatments for neurodegeneration such as surgery. In some embodiments, the compounds described herein may be combined with treatments for cardiomyopathy such as surgery.

The term “derivative” as applied to a phosphate containing, monophosphate, diphosphate, or triphosphate group or moiety refers to a chemical modification of such group wherein the modification may include the addition, removal, or substitution of one or more atoms of the phosphate containing, monophosphate, diphosphate, or triphosphate group or moiety. In some embodiments, such a derivative is a prodrug of the phosphate containing, monophosphate, diphosphate, or triphosphate group or moiety, which is converted to the phosphate containing, monophosphate, diphosphate, or triphosphate group or moiety from the derivative following administration to a subject, patient, cell, biological sample, or following contact with a subject, patient, cell, biological sample, or protein (e.g., enzyme). In an embodiment, a triphosphate derivative is a gamma-thio triphosphate. In an embodiment, a derivative is a phosphoramidate. In some embodiments, the derivative of a phosphate containing, monophosphate, diphosphate, or triphosphate group or moiety is as described in Murakami et al. J. Med Chem., 2011, 54, 5902; Sofia et al., J. Med Chem. 2010, 53, 7202; Lam et al. ACC, 2010, 54, 3187; Chang et al., ACS Med Chem Lett., 2011, 2, 130; Furman et al., Antiviral Res., 2011, 91, 120; Vernachio et al., ACC, 2011, 55, 1843; Zhou et al, AAC, 2011, 44, 76; Reddy et al., BMCL, 2010, 20, 7376; Lam et al., J. Virol., 2011, 85, 12334; Sofia et al., J. Med. Chem., 2012, 55, 2481, Hecker et al., J. Med. Chem., 2008, 51, 2328; or Rautio et al., Nature Rev. Drug. Discov., 2008, 7, 255, all of which are incorporated herein by reference in their entirety for all purposes.

As used herein, the term “animal” includes, but is not limited to, humans and non-human vertebrates such as wild, domestic, and farm animals.

As used herein, the term “antagonize” or “antagonizing” means reducing or completely eliminating an effect.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “individual,” “subject,” or “patient,” used interchangeably, means any animal, including mammals, such as mice, rats, other rodents, rabbits, dogs, cats, swine, cattle, sheep, horses, or primates, such as humans.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “prodrug” means a derivative of a known direct acting drug, which derivative has enhanced delivery characteristics and therapeutic value as compared to the drug, and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the disclosure can optionally exclude one or more embodiment for purposes of claiming the subject matter.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

a. Methods of Treating a PSP

In some embodiments, compounds and compositions described herein are useful in treating a PSP. Thus, provided herein are methods of treating a PSP, comprising administering to a subject in need thereof, a therapeutically effective amount of a compound as described herein (e.g., modulators of COX, dopamine receptors, sodium channels, serotonin receptors, acetylcholine receptors, GABA receptors, FAAH, adrenergic receptors, histamine receptors, vasopressin receptors, NMDAR, beta amyloid, gamma-secretase, IxB/IKK, glutamate receptors, opioid receptors, TRPV, aldose reductase, calcium channels, glucocorticoid receptors, and/or HMG-CoA reductase) or a pharmaceutically acceptable salt thereof, or a composition comprising a disclosed compound or pharmaceutically acceptable salt thereof. Disorders treatable by the present compounds and compositions include, e.g., achalasia, Hirschsprung's disease, an intestinal pseudo-obstruction, gastroesophageal reflux disease (GERD), functional dysphagia, functional dyspepsia, irritable bowel syndrome (IBS), gastroparesis, functional constipations, functional diarrhea, and fecal incontinence.

In some embodiments, the disclosure relates to any of the above disclosed methods disclosed herein, wherein the administrating step comprises administering a pharmaceutical composition comprising: (i) a pharmaceutically effective amount of any of the disclosed compounds; and (ii) a pharmaceutically acceptable carrier.

Thus, in various embodiments, disclosed are methods for treating a PSP in a subject in need thereof, the method comprising administering to the subject an effective amount of a compound selected from carprofen, mefenamic acid, phenacetin, valdecoxib, fenoldopam mesylate, fluphenazine hydrochloride, bupivacaine HCl, phenazopyridine HCl, alverine citrate, nitenpyram, 4-aminobutyric acid (GABA), PF-3845, esmolol HCl, cimetidine, conivaptan HCl, (+)-MK-801 maleate, MK-0752, R04929097, rosmarinic acid, theophylline, aripiprazole, flopropione, latrepirdine 2HCl, ADX-47273, MPEP, nefopam HCl, phenazopyridine HCl, epinephrine, (+)-matrine, phenothiazine, naproxen sodium, AMG-517, isoliquiritigenin, nilvadipine, prednisone, and simvastatin, or a pharmaceutically acceptable salt thereof. In a further embodiment, the compound is selected from aripiprazole, dexmedetomidine, matrine, and MPEP, or a pharmaceutically acceptable salt thereof. In still further embodiments, the compound is dexmedetomidine.

In further embodiments, the compound is FDA approved.

In further embodiments, the administering is accomplished by oral adminstration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

In various embodiments, the method further comprises administering an effective amount of an agent associated with the treatment of a PSP.

Thus, in various embodiments, the method further comprises administering an agent known for the treatment of a PSP. Examples of agents known for the treatment of PSPs include, but are not limited to, parasympathomimetics, prokinetic agents, opioid antagonists, antidiarrheals, and antibiotics. In further embodiments, the agent is selected from neostigmine, bethanechol, metoclopramide, cisapride, and loperamide.

In some embodiments, the compound and the agent are administered simultaneously. In some embodiments, the compound and the agent are administered sequentially.

In some embodiments, the compound and the agent are co-packaged. In some embodiments, the compound and the agent are co-formulated.

### B. Methods of Neutralizing Neurotoxicity in a Subject

In some embodiments, disclosed are methods of neutralizing neurotoxicity in a subject, the method comprising the step of administering to the subject a therapeutically effective amount of at least one disclosed saxiphilins, or a pharmaceutically acceptable salt thereof.

Thus, in various embodiments, disclosed are methods for modulating neurotoxicity of a toxin in a subject having PSP, the method comprising administering to the subject an effective amount of an amino acid disclosed herein, or a pharmaceutically acceptable salt thereof. In a further embodiment, the compound is selected from a functional fragment of a saxiphilin or a pharmaceutical composition free of SEQ ID NO: 1, SEQ ID NO:2, SEQ ID NO:3, SEQ ID NO:4, SEQ ID NO:5, SEQ ID NO:6, SEQ ID NO: 7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15 or a pharmaceutically acceptable salt thereof.

In further embodiments, neutralization of the toxin prevents death of the subject.

As used herein, “modulation” can refer to either inhibition of toxicity when a subject is exposed to a toxin. For example, the modulation of neurotoxicity can refer to the inhibition of toxin, In some embodiments, the compounds described herein increase survival from exposure to STX or a STX family toxin by a factor from about 1% to about 50%. The survival can be measured by any method including but not limited to the methods described herein.

In further embodiments, modulating is inducing neutralization of toxin.

In further embodiments, the subject is a mammal. In still further embodiments, the subject is a human.

In further embodiments, the subject has been diagnosed with a need for treatment of a PSP prior to the administering step. In still further embodiments, the method further comprises the step of identifying a subject at risk of developing a PSP prior to the administering step.

In further embodiments, the administering is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

In various embodiments, the method further comprises administering an effective amount of an agent associated with the treatment of PSP.

Thus, in various embodiments, the method further comprises administering an agent known for the treatment of PSP. Examples of agents known for the treatment of PSP include, but are not limited to, parasympathomimetics, prokinetic agents, opioid antagonists, antidiarrheals, and antibiotics. In further embodiments, the agent is selected from neostigmine, bethanechol, metoclopramide, cisapride, and loperamide.

In some embodiments, the compound and the agent are administered simultaneously. In some embodiments, the compound and the agent are administered sequentially.

### C. Pharmaceutical Compositions

Also provided herein are pharmaceutical compositions comprising an amino acid sequence or saxiphilin as disclosed herein, or pharmaceutically acceptable salt thereof; and a pharmaceutically acceptable carrier. Thus, in various embodiments, disclosed are pharmaceutical compositions comprising a therapeutically effective amount at least one disclosed amino acid sequence or functional fragment thereof (e.g., SEQ ID NO: 1 through SEQ ID NO:15, and a pharmaceutically acceptable carrier. In a further embodiment, a pharmaceutical composition can be provided comprising a therapeutically effective amount of at least one disclosed compound. In a still further embodiment, a pharmaceutical composition can be provided comprising a prophylactically effective amount of at least one disclosed compound. In yet a further embodiment, the disclosure relates to pharmaceutical compositions comprising a pharmaceutically acceptable carrier and a disclosed compound, wherein the compound is present in an effective amount. In an even further embodiment, the pharmaceutical compositions are useful in inhibiting neurotoxicity in a subject. In a still further embodiment, the pharmaceutical compositions are useful in treating PSP.

Thus, in various embodiments, provided herein are pharmaceutical compositions comprising a therapeutically effective amount of a saxiphilin, analogs and functional fragments of a saxiphilin, or salts thereof. In some embodiments, the amino acid sequences of the disclosure relate to an American Bullfrog saxiphilin sequence (SEQ ID NO:1), or functional fragments and variants thereof. SEQ ID NO:1:

MAPTFQTALFFTIISLSFAAPNAKQVRWCAISDLEQKKCNDLVGSCNVP
DITLVCVLRSSTEDCMTAIKDGQADAMFLDSGEVYEASKDPYNLKPIIA
EPYSSNRDLQKCLKERQQALAKKMIGHYIPQCDEKGNYQPQQCHGSTGH
CWCVNAMGEKISGTNTPPGQTRATCERHELPKCLKERQVALGGDEKVLG
RFVPQCDEKGNYEPQQFHGSTGYSWCVNAIGEEIAGTKTPPGKIPATCQ
KHDLVTTCHYAVAMVKKSSAFQFNQLKGKRSCHSGVSKTDGWKALVTVL
VEKKLLSWDGPAKESIQRAMSKFFSVSCIPGATQTNLCKQCKGEEGKNC
KNSHDEPYYGNYGAFRCLKEDMGDVAFLRSTALSDEHSEVYELLCPDNT
RKPLNKYKECNLGTVPAGTVVTRKISDKTEDINNFLMEAQKRQCKLFSS
AHGKDLMFDDSTLQLALLSSEVDAFLYLGVKLFHAMKALTGDAHLPSKN
KVRWCTINKLEKMKCDDWSAVSGGAIACTEASCPKGCVKQILKGEADAV
KLEVQYMYEALMCGLLPAVEEYHNKDDFGPCKTPGSPYTDFGTLRAVAL
VKKSNKDINWNNIKGKKSCHTGVGDIAGWVIPVSLIRRQNDNSDIDSFF
GESCAPGSDTKSNLCKLCIGDPKNSAANTKCSLSDKEAYYGNQGAFRCL
VEKGDVAFVPHTVVFENTDGKNPAVWAKNLKSEDFELLCLDGSRAPVSN
YKSCKLSGIPPPAIVTREESISDVVRIVANQQSLYGRKGFEKDMFQLFS
SNKGNNLLFNDNTQCLITFDRQPKDIMEDYFGKPYYTTVYGASRSAMSS
ELISACTIKHC

In some embodiments, the one or plurality of saxiphilins are chosen from an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or a variant that comprises at least about 70%, 80%, 87%, 90%, 95%, 96%, 97%, 98%, or 99% sequence identity to the amino acid sequences above.

TABLE 3
Saxiphilin Sequences

| SEQ | | | Common | |
|---|---|---|---|---|
| ID NO. | Saxiphilin | Organism | names | Amino acid sequence |
| 1 | RcSxph | Rana | American | MAPTFQTALFFTIISLSFAAPNAKQVRWCAISDLEQKKCNDLVG |
| | | (Lithobates) | bullfrog, | SCNVPDITLVCVLRSSTEDCMTAIKDGQADAMFLDSGEVYEAS |
| | | catesbeianus | Bullfrog | KDPYNLKPIIAEPYSSNRDLQKCLKERQQALAKKMIGHYIPQCD |
| | | | | EKGNYQPQQCHGSTGHCWCVNAMGEKISGTNTPPGQTRATCE |
| | | | | RHELPKCLKERQVALGGDEKVLGRFVPQCDEKGNYEPQQFHG |
| | | | | STGYSWCVNAIGEEIAGTKTPPGKIPATCQKHDLVTTCHYAVA |
| | | | | MVKKSSAFQFNQLKGKRSCHSGVSKTDGWKALVTVLVEKKLL |
| | | | | SWDGPAKESIQRAMSKFFSVSCIPGATQTNLCKQCKGEEGKNC |
| | | | | KNSHDEPYYGNYGAFRCLKEDMGDVAFLRSTALSDEHSEVYE |
| | | | | LLCPDNTRKPLNKYKECNLGTVPAGTVVTRKISDKTEDINNFL |
| | | | | MEAQKRQCKLFSSAHGKDLMFDDSTLQLALLSSEVDAFLYLG |
| | | | | VKLFHAMKALTGDAHLPSKNKVRWCTINKLEKMKCDDWSAV |
| | | | | SGGAIACTEASCPKGCVKQILKGEADAVKLEVQYMYEALMCG |
| | | | | LLPAVEEYHNKDDFGPCKTPGSPYTDFGTLRAVALVKKSNKDI |
| | | | | NWNNIKGKKSCHTGVGDIAGWVIPVSLIRRQNDNSDIDSFFGES |
| | | | | CAPGSDTKSNLCKLCIGDPKNSAANTKCSLSDKEAYYGNQGAF |
| | | | | RCLVEKGDVAFVPHTVVFENTDGKNPAVWAKNLKSEDFELLC |
| | | | | LDGSRAPVSNYKSCKLSGIPPPAIVTREESISDVVRIVANQQSLY |
| | | | | GRKGFEKDMFQLFSSNKGNNLLFNDNTQCLITFDRQPKDIMED |
| | | | | YFGKPYYTTVYGASRSAMSSELISACTIKHC* |
| 2 | NpSxph | Nanorana | High | MALTFHTALYFTIVGLSFAASDARHVQWCTISHLEQKKCNDLV |
| | | parkeri | Himalaya | GSCNVPDITLACVYRSSTENCMAAIKDGQADAMFLDSGDVYK |
| | | | frog. | ASLDHYNLKPIIAEPYSLHRELTKCLKHRQESLGGDKMVKGRYI |
| | | | Xizang | PQCDEKGNYHPVQCHASTGYCWCVNANGEKIEGTNTTPVQTP |
| | | | Plateau | PTCPSQVLTKCLKERQEALGGKRIAIGRYIPQCDEQGNYRPMQ |
| | | | frog. | CHGSTGYCWCVNAIGEKIEGTNTPPGNTQPTCQSHDWDTCHY |
| | | | Parker's | AVAVVKNSSTFQFGQLKGKRSCHSGLSKTDGWNAPVNVFVEK |
| | | | slow frog, | KLLPWDGLAKGSIERAVSKFFSASCIPGATETNLCKQCIGEEEK |
| | | | mountain | KCKSSHDEPYYGDHGAFRCLQEDKGDVAFLKNTALPDEHSGV |
| | | | slow frog | YELLCPDNTRKPLNKYKECNLGKVPADAVVTRKAGDKTKDIN |
| | | | | DFLLEAQKKKCKLFGSPHGKDLMFDDSTTHLAPLPSEIDAFFFL |
| | | | | GVKWYNAMKALTEDVKLPSKNKVRWCTINKPEMMKCKDWA |
| | | | | AVSGGAIACTEASCPEHCVKQILKGEADAVTLDVQYMYMALM |
| | | | | CGLLPAVEEYPNKDDFHPCQIPGSTIKDFGTKRAVALVKKSNK |
| | | | | DIKWNNLKGKKSCHTHVGDIPGWVIPAGLISNQNDNIDIESFFG |
| | | | | ESCAPGSDTNSKLCKLCIGDPENPKASTRCSLSDKEAYYGNEG |
| | | | | AFRCLVEKGDVAFVPHTVVFANTDGKNPAEWAKDLKSEDFEIL |
| | | | | CLDGSRAPVTNYRGCNLSGLPPRAIVTREESVSDVVRILINQQS |
| | | | | LYGRNGFEKDMFQMFSSAKGQNLLFNDETQCLIEFDRQPKDIM |
| | | | | EDYFGVRYYTAVYSASRSAVPSELIPACTFKHC* |
| 3^{†} | MaSxph | Mantella | Golden | MALTFQTALYFTIIGLSFVTSSARDVRWCVISDLEQKKCNDLVG |
| | | aurantiaca | mantella | SCKVSGITLVCVHRSSTENCMTAIRDEQADAMFLDSGDVYKAS |
| | | | | MSPYNLKPIIAESYSSHKECLKKRQEALGGANVLGQFVPQCDE |
| | | | | KGSYQPQQCYGSTGHCWCVNAIGEEIVGTKTQPGQTQATCER |
| | | | | HDQTKCLKDRQKALAKKMIGGFVPQCDEKGNFKPRQCHGSTG |
| | | | | YCWCTNANGEKIAATNTPPQQSPPTCERHDLDSCHYAVAVVK |
| | | | | KSSIFQFNQLKGKRSCHSGVSKTDGWKAPVSVLVDKNLLSWD |
| | | | | RAAKESIEKAVSKFFSDSCIPGATETNLCNQCMGEGGKKCMSS |
| | | | | HDEPYYGDHGAFRCLKEDKGDVAFLKNTVLSDTHSEDYELLC |
| | | | | PDNTRKPLNKYKECNLGKVPADAVVTRKSGDKIKDINDFLLEA |
| | | | | QKKHCQLFSSAHGKDLMFDDSTTQLSLLPPEVDVFFFLGVQWL |
| | | | | NTMKTLTADVKPPSKNKVRWCTVNNLEKMKCDDWSAVSGGA |
| | | | | IECTEASSPEHCIKQIVKGEADAVTLDVQYMYEALKCGLLPAVE |
| | | | | EYHNKDDFGPCKTPGSEIKDFGAKRAVALVKKSNKDINWHNL |
| | | | | KGKKSCHTGVGDQAGWIIPVGLISRQIDNCNIDSFFSESCAPGSD |
| | | | | TTSNFCKLCIGDPSNPMANTKCSLSDKEAYHGNEGAIRCLVEK |
| | | | | GDVAFVHHTAVFENTDGKNPAEWAANLKSEDFEILCLDGSRAP |
| | | | | VSNYMTCRLSGVPPRAIVTREESVSDVVRILINQQSLFGRNGFE |
| | | | | KDMFQMFSSNKGQNLLFNDGTQCLIEFDRQPRGIMEDYFGKPY |
| | | | | YNAVYNTSKCVLPSELTSACTFKHC* |
| 4^{†} | DtSxph | Dendrobates | Dyeing | MRMTPQVLLWLCLLALSSAAPNIRNVRWCVTSDTEEQKCNDL |
| | | tinctorius | poison dart | VTSCHVDEILLTCVKKSSTEDCVRAISNGEADAISLDSKDVYKA |
| | | | frog, | SLDPFNLKPIMTEAYSEREHTPCMRHRQSVLGGKNMKIGAFVP |
| | | | Dyeing dart | KCDEKGNYVPKQCHGSTGYCWCLNENGEEIEGTRTPPGNSGLT |
| | | | frog | CENKANKPPCLKERQKLLSAKPSPAVFVPECDEKGNYRPQQSH |
| | | | | VYTWCVDEYGEEVFGSRNFPGKPPKPCEASGETACIKERNKVL |
| | | | | SVAEPLLGAFLPDCDENGYFSPLQFHGSTGYSWCVTKNGEEIK |
| | | | | GTRTGPGQTPPTCEVSAPVTLHYAVAVVKKSSSFQLDQLKGKR |
| | | | | SCHSAVGEAAGWVAPLNVLLKKKLLLWEEPEQKSIEKVASEIF |
| | | | | SASCAPGAQEANLCEQCAGQEDQCTRGPGEPYYGDEGAFRCL |
| | | | | RDGKGDVAFVEDTALTGQYSDNFELLCPDNTRRPLSQYKICNF |
| | | | | GRIPRHSVVTRSTGDKMQDITEYLLQAQKKECKLFSSTHGKDL |
| | | | | LFEDTTSALIALPSAMDTFLFLGPDLFNGMKTLNGARPPRVKQ |
| | | | | QIRWCPQSKNEHKKCEDWSSVSGGAIKCTEPSSALECVKKILK |
| | | | | DEADAVNLDTAHTYTALKCGLLPSLDEYRNKDDLIPCQIPGAD |
| | | | | YTEFGSYRIVAIVKKKDKDITWNNLQGKKSCHTGVGDMMGW |
| | | | | NIPVYLISKKTKNCDLGSYFSQSCAPGSPIDSNLCKLCIGDPQNT |
| | | | | EANTKCSLSDKEAYYGNEGAVRCLVEKGDVAFVPHTAVFENT |
| | | | | DGKNPALWAKDLKSADFELLCPDGSRAPVSNYKTCKLSGIAN |
| | | | | QVTVTRPESVKDVVRITQNQQSLYGRTGSLKDIFQMFSTSYGQ |
| | | | | NLLFSDRTQCLVEFDRMLDRDIMDDYFGKPFHKHLIRDNDCFPI |
| | | | | SALATACSFHH* |
| 5^{†} | OsSxph | Oophaga | Diablito, | MRMTLQVLLCLCLLVLSSAAPNSRNVRWCVTSDTEEQKCNDL |
| | | sylvatica | Little devil | VTSCHVDEILLICVKKSSKEDCVRAISNGEADAISLDSKDVYKA |
| | | | poison frog | SLDPFNLKPIMTEAYSESEQTPCMRHRQSVLGGKKMIIGAFVPK |
| | | | | CDEKGNYVPKQCHDSTGFCWCLNENGEEIEGTRTPPGNSGLTC |
| | | | | ENKANKPPCLKERQKLLSAKPSPAVFVPECDEKGNYRPEQSHV |
| | | | | YSWCVDEYGEEVLGSRTFPGKPPKPCEASGETPCIKERNKVLSA |
| | | | | AEPLPGAFVPDCDEKGYFSPLQFHGSTGHSWCVTKNGEEIKGT |
| | | | | RTRPGQTPPTCDVPAPATLHYAVAVVKKSSSFQLDQLKGKRSC |
| | | | | HSAVGEAAGWVAPLNVLLKKKFLLWEEPEQKSTEKVASEIFSA |
| | | | | SCAAGAQEANLCEQCAGQEDKCTRGPGEPYYGDEGAIRCLRD |
| | | | | DKGDVAFVEDTALTGQYSDNFELLCPDNTRRPLSQYKICNFGS |
| | | | | VPRHAVVTRSTGDKMKEITEYLLQAQKKECKLFSSTHGKDLLF |
| | | | | EDTTSALIALPSGMDTFLFLGPDLFNGMKTLNGAHPPRVKQKIR |
| | | | | WCPQNKNEKKKCDDWSSVSGGSIKCTEPSSALECIKMILKDEA |
| | | | | DAVNLDAEHAYTALKCGLLPSLEEYRNKDDLIVCQIPGAEYSE |
| | | | | FGSYRIVALVKKTDKDITWNNLQGKKSCHTHVGDMIGWNIPV |
| | | | | YLISKKTKNCDLGSYFSQSCAPGSPIDSNLCKLCIGDPQNTEANT |
| | | | | KCSLSDKEAYYGNEGAVRCLVETGDVAFVPHTAVFENTDGKN |
| | | | | PALWAKDLKSTDFELLCPDGSRAPVSDYKKCKLSGIANHVTVT |
| | | | | RPESVKDVVRITQNQESLYGRTGSQKDIFQMFSSSYGQNLLFND |
| | | | | RTQCLFEFDRMLDRDIMDDYLGKPFHKHLMSDNDCLPKSALA |
| | | | | TACSFHH* |
| 6^{†} | RiSxph | Ranitomeya | Mimic | MRRSSYAASQEMRMTLRVLLCLCLWALSSAAPNTRNVRWCV |
| | | imitator | poison | TSDTEEQKCNDLVTSCHVDEILLICVKKSSTEDCVRAISNGEAD |
| | | | frog. | AISLDSKDVYKASLDPFNLKPIMTEAYSESEHTPCMRHRQSVLG |
| | | | Poison | GKIMKIGAFVPKCDEKGNYVPKQCHGSTGYCWCLNENGEEIEG |
| | | | arrow frog | TRTPPGNPGLTCENKANKPPCLKERQKLLSAKPSPAVFVPECDE |
| | | | | KGNYRPQQSHVYSWCVDEYGEEVFGSRSFPGKPPKPCEASGET |
| | | | | PCIKERNKVLSAAEPLLGAFVPDCDEKGYFSPLQSHGSTGYSW |
| | | | | CVTKNGDEIKGTRTGPGQSPPTCEVPAPVTLHYAVAVVKKSSS |
| | | | | FHLDQLKGKRSCHSAVGEAAGWVAPLNVLLKKKLLLWEEPEP |
| | | | | KTIEKVASEIFSASCAPGAQEANLCEQCAGQEDKCTRGPGEPYY |
| | | | | GDEGAFRCLREDKGDVAFVEDTALTGQYSDNFELLCPDNTRRP |
| | | | | LSQYKICNFGRIPRHAVVTRSSGDKMKDITEYLLQAQKKECKLF |
| | | | | SSTHGKDLLFEDTTSALIALPSAMDTFLFLGPDLFNGMKTLNGA |
| | | | | RPPQVNQQIRWCPQSKNEKKKCDDWSSVSGGAIKCTEPSSGLE |
| | | | | CIKMILKGEAEAVNLDIEHSYIALKCGLLPSVDEYRNKDDLIPC |
| | | | | QIPGSEYSEFGSYRIVALVKKTDKDLTWNNLQGKKSCHTRAGD |
| | | | | MIGWNIPVYLISKKTKNCDLGSYFSQSCAPGSPIDSNLCKLCIGD |
| | | | | PQNPQANTKCSISDKEAYYGHEGAIRCLVEKGDVAFVPHTAIFE |
| | | | | NTDGKNPALWAKDLKSTDFELLCPDGSRAPVNDYKKCKLSGI |
| | | | | ANQIAITRPESVKDVVRIIQNQQSLYGRTGSQKDIFQMFSSSYG |
| | | | | QNLLFSDRTQCLVEFDRMLDRDIMDDYFGKPFHKHLLRDNDC |
| | | | | LPKSALASACSFHH* |
| 7^{†} | PtSxph | Phyllobates | Golden dart | MRMTTAFQVLLCVCLLALSSAAPNARNVRWCVTSDAEEQKCN |
| | | terribilis | frog, | DLVTSCHVDEILLTCVKKYSTEDCVRAISNGEADAISLDSKDVY |
| | | | Golden | KASLHPFNLKPIVTEAYSEKEHTPCMRHRQSVLGGKKIKIGAFV |
| | | | poison | PKCDEKGNYVPKQCHGSTGYCWCLNENGEEIEGTRTPPGTKHL |
| | | | frog, | TCEDTADKPPCLKKRQKLLSAKPSPAVFVPECDEKGNYRPEQS |
| | | | Golden | HVYTWCVDEYGEEVFGSRNFPGKPPKPCVASDEPLCIKERNKV |
| | | | | LGTTEPLLGAFVPDCDEKGYFSPLQFHGSTGYSWCVTENGVEI |
| | | | poison | KGTRTGPGQTPPTCEVPAPMTLNYAVAVVKKSSSFHLDQLKG |
| | | | arrow frog | KRSCHSAVGEAAGWVAPLNVLLKKKLLSWGGPEQKSIEKVAS |
| | | | | EIFSASCAPGAQEANLCEQCAGQEDKCTRGPGEPYYGDEGAFR |
| | | | | CLRDDKGDVAFVEDTVLSGQYSDNFELLCPDNTRRPLSQYKIC |
| | | | | NFGRIPRHAVVTRTTGDKMKDITEYLVQGQKKECKLFSSTHGK |
| | | | | DLLFEDTTTALITLPSAMDTFFFLGSELFKGMKTLEGTRPPRVH |
| | | | | REIRWCPQNKNEKKKCDDWSSVSGGAIKCTEPSSALECIEMILK |
| | | | | GEAEAVNLDAEHAYTALKCGLLPSVEEYRNKDDLIPCQVPGAE |
| | | | | YSEFGGNRIVALVKKTDKDITWNDIQGKKSCHTHVGDMIGWNI |
| | | | | PIYLIYKKTKNCDIGSFFSQSCAPGSTIDSNLCKLCIGDPQNPEAN |
| | | | | TKCSLSDKEAYYGNEGAIRCLVEKGDVAFVPHTAVYENTDGK |
| | | | | NPALWAKDLKSTDFELLCLDGSRAPVSDYKKCKLSGIVNQVTV |
| | | | | TRPESVKDVVRIIHNQESLYGRTGFQKDIFQMFSSSYGENLLFS |
| | | | | DRTQCLFEFDRMLDRDIMEDYLGKPFHKYVMRDNECLPKSAL |
| | | | | ATACSFHH* |
| 8^{†} | EtSxph | Epipedobates | Phantasmal | MVSSRKLRMTSAFQLLLCLCLMVLSFAAPNARNVRWCVTSDA |
| | | tricolor | poison | EEHKCNDLVNSCHVNDILLKCVKKSSTEDCVRAISNGEADTISL |
| | | | frog, | DSKDVYKASLHPFNLKPIMTEGYSEREHTPCMRHRQSVLEGKK |
| | | | Phantasmal | MKIGAFVPKCDEKGNYAPKQCHGSSGYCWCLNENGEEIEGTR |
| | | | poison- | TPPGTKSLTCEDAANKPPCLKERQKLLSSKPAPTVFVPECDEKG |
| | | | arrow frog | NYRPQQCHIYCWCVDEYGEEVLFSRSFPGKPPKKCEPSGETLCL |
| | | | | KQRNKVLSATEPLLGTFVPECDDKGYFTPQQFHGSTGYSWCVT |
| | | | | KNGDEIPGTRTGPGQTPHICEVKAPVTLHYAVAVVKKSSSFQL |
| | | | | DELKGKRSCHSAVGEAAGWVAPLKILLKKKLLSLEGAEVKSIE |
| | | | | KAASEIFSASCAPGAHEAKLCEQCAGQEGKCHRGPGEPYYGDE |
| | | | | GAFRCLRDNKGDVAFVEDTALSGQYSDNYELLCPDNTRRPLSQ |
| | | | | YKICNFGRVPRHAVVTRSSGDKMKDITEYLLEAQKKECKLFSS |
| | | | | THGKDLLFEDTTTGLIALPSAMDTFLFLGPELFHGMKTLDGAHP |
| | | | | PSSQEIRWCPLTKNEKKKCDDWSSVSGGAIKCTEPSSGQKCIEK |
| | | | | ILKGEADVVTLGVEHAYTALKCGLHISFEEYRNKDDLIPCQVLE |
| | | | | AEYTEFGSYRIVAVVKKTDKDITWNNLQGKKSCHTSVNDMVG |
| | | | | WKIPHALLYKKTKSCDFGSYFSKSCAPGSAIDSNLCKLCIGDPQ |
| | | | | NTKSNTKCSLSDKEAYYGNDGAIRCLVEKGDVAFVPHTAIFEN |
| | | | | TDGKNPALWAKDLKSTDFELLCLDGSRAPVSDYKTCKLSGIAN |
| | | | | QVTVNRPERQADVLRIILNQQSLYGRTGSQKDIFQMFSSSYGQN |
| | | | | LLFNDRTQCLFVFDRMLDRDIVDDYIGKAMQRDVLRDNECFP |
| | | | | QTALTNACLFHH* |
| 9^{†} | AfSxph | Allobates | Brilliant- | MRMTPALLGLLCLCLLVLSSAAPNSRTVRWCVTSDTEQQKCN |
| | | femoralis | thighed | DLVNSCHVDEIVLSCVKKSNTEDCVRAISTGEADAISLDSKDVY |
| | | | poison | KASLDPFHLKPIMTEGYSERDHTPCMKHRQSVLGGKQLIIGAFV |
| | | | frog, | PKCDEKGNYAPKQCHGSSGYCWCLNENGEEIKGTRSRPGTKV |
| | | | Brilliant- | LTCEDAANKPPCLKERQKLLSENPSPTVFVPECDEKGNYRPRQ |
| | | | thighed | CHDYCWCVDEYGEEIFGSRTFPGKPPKACEASGETLCIKERNK |
| | | | poison | VLSTAEPLRGAFLPECDEKGYFSPMQCHSSTGHCWCTTKDGEE |
| | | | arrow frog | IEGTRTGPGQSRPTCDIPAPGTVHYAVAVVKKSSNFQLDGLKG |
| | | | | KRSCHSAVGESGGWVAPLNIFLKKNLLSWEGPEQKSIEKAASE |
| | | | | FFSASCAPGAQESTLCEQCAGQEDKCKRGPGEPYYGDEGAIKC |
| | | | | LKDDKGDVAFVEDTILSGQYSNDFELLCSDNTRRPLSQYKMCN |
| | | | | FGKIPRHAVVTRSSGDKMEDITEYLLKAQKKECKLFGSAVGKD |
| | | | | LLFEDTTTSLIVLPSAMDTFLYLGPELFSGMKTLEGAHPPSNQEI |
| | | | | RWCPQSKNEKKKCDDWSSVSGGAIKCTEPSSALECIEKILKGEA |
| | | | | EAVTLDGEHAYTALKCGLVPSLDEYRNKDDLVPCQIPGAEYSE |
| | | | | FGSYRIVALVKKTDKDITWDNLQGKKSCHTSVGDMMGWTVPI |
| | | | | SLIYKKTKNCDIGSYFSQSCAPGSAIDSNLCELCIGDPGNPTANT |
| | | | | KCSQNDKEAYYGNEGAIRCLVEKGDVTFVPHTAVYENTDGKN |
| | | | | PALWAKDLKSTDFELLCPDGSRAPVSDYKRCKLSGIANQITVT |
| | | | | RSESLKDVVRIILNQQSLYGRNGFQKDIFQLFSSVHGQNLLFSD |
| | | | | RTQCLVEFDRMLDRDIMDDYFGKPFHKTVFRDNDCLPKSALA |
| | | | | TACSFHH* |
| 10^{†} | RmSxph | Rhinella | Marine | MTPAFQVLLCLCLLGMSYAAPQARNVRWCVTSDAEEEKCNDL |
| | | marina (Bufo | toad, | VNTCTVKEILLICVKKSSTEDCIRAISAGEADAISLDSEDIYQASL |
| | | marinus) | South | KPFHLKPIMTETYASKKNIKLNQSPCQKEHQHLLEGGPKCDETE |
| | | | American | STPCLRHRQRVLGARKPQIGEFVPECDEKGNYFPKQCYGTTGY |
| | | | cane toad, | CWCVDEHGDEIPVGRAKQGKVNISCEYAASEKPCMKEQRKAL |
| | | | Cane toad, | SGGQPLRGAFIPNCDEKGNYSPKQCHGSTGYCWCVDENGAEIS |
| | | | Giant toad | GSRTPPGQQVPTCGSYVGATCIKDRYKVLGAGKPLPGAFVPDC |
| | | | | DEKGDYRPQQCHGSTGHCWCVSKDGVEIQGTRAAPGQSPPTC |
| | | | | EDPDLVKSHYAVAVVKKSSTLQFNHLKGKRSCHSAVGKTAGW |
| | | | | IAPLYALYKKHFLLWDGSEEKSFEQAASEFFSASCAPGAKEENL |
| | | | | CKQCAGQEDNCKSSPGEPYYGDEGALRCLREDKGDVAFVEHT |
| | | | | ALSGKDSDNFELLCPDNTRSPVSEHKDCNFGKVPRHAVVTRDT |
| | | | | GDKSKDIIEYLLEAQKKECKLFSSTCGKDLMSGDTTTNLIALPS |
| | | | | GMDAFLFLGPELFNAMKTLHGAPLPSKNEILWCTQNKNEKTKC |
| | | | | DDWSAVSGGAIKCTEPLSADQCIEKILKREADAVTLHTEHMYT |
| | | | | ALKCGLVPAVEGYYNKDDFTPCKTDGDFYKDFGGTYVVALVK |
| | | | | KSDKDITWNNLKGKKSCHTGVGHWTGWMIPVSQISKQTKNCD |
| | | | | IGSYFSQSCAPGSAIDSNLCELCIGDQQNTKAQTQCSPSDKEAY |
| | | | | YGGAGAIRCLVEKGDVAFVTDRDVFKNTDGNNPALWAKDLK |
| | | | | STDFELLCLDGSRAPVTDYKNCNLPGNPNLSVVSREENVNDVV |
| | | | | RIFLNQQSLYGRNGSQKDKFQMFSSYGENLLFSDDAQCMIEFD |
| | | | | MIKGRDLIDDVLGKHFSATMLRNNPCLPKSELRTACTFRAEKI |
| 11 | BbSxph | Bufo | Common | MKHSSSSAQKEVRMTPAFQVLLCLCLLGMSYAAPQARNVRW |
| | | | toad, | CVTSDAEEEKCNDLVNSCPVKEILLICVKKSSIEDCMRAISAGE |
| | | | Caucasian | ADAISLDSENIYKASLKPFHLKPIMTETYISKKDIKLTQGPCQKE |
| | | | toad | HQHLLEGRQHKCEETESTPCLRHRQRVLGAKKPHIGEFMPECD |
| | | | | EKGNYFPKQCYGTTGYCWCVDENGAERHVGRAKQGKVNISC |
| | | | | EYTESQKPCMKERRKVLSGGQPLPGAFIPDCDEEGNYNPKQCH |
| | | | | GSTGYCWCVDENGAEISGSRMPTGQSDPTCGTYAGATCIKDRY |
| | | | | KVLGAGEALRGAFVPDCDEKGDYRSKQCHGSTGYCWCVSKY |
| | | | | GVEIQGTRTAPGQSPTTCEIPGGEKPCMKERRKSLSGGQPLPGA |
| | | | | FMPDCDEKGNYSPKQCHGSTGYCWCVNENGKEISGSRTPPGQ |
| | | | | QVPTCVASVLSAETSCIKERQKVLGAVKPILGAFVPDCDEKGEY |
| | | | | RPKQCHGSTGYCWCVSKDGKEIQGTRAAPGQSPPTCQDTGEK |
| | | | | LVLSAETSCIKERQKVLGAVKPILGAFVPDCDEKGEYRPKQCH |
| | | | | GSTGYCWCVSKDGKEIQGTRAARGQSPPTCQDTGEKLVLSAET |
| | | | | SCIKELQKVLGAVKPMVGAFVPDCDEKGEYRPKQCHGSTGYC |
| | | | | WCVSKDGKEIQGTRVAPGQSPPTCQDTVLSAETSCIKERQKVL |
| | | | | GAVKPILGAFLPDCDEKGEYRPKQCHGSTGYCWCVSKDGKEIQ |
| | | | | GTRAAPGQSPPTCQDTGEKLVLSAETSCIKERQKVLGAVKPIVG |
| | | | | AFVPDCDEKGEYRPKQCHGSTGHCWCVSKDGKEIQGTRVAPG |
| | | | | QSPPTCQDTVLSAETSCIKERQKVLGAVKPILGAFLPDCDEKGE |
| | | | | YRPKQCHGSTGYCWCVSKDGKEIQGTRAAPGQSPPTCQDTVLS |
| | | | | AETSCIKERQKVLGAVKPILGAFLPDCDEKGEYRPKQCHGSTG |
| | | | | YCWCVSKDGKEIQGTRAAPGQSPPTCQDTGEKLVLSAETSCIK |
| | | | | ERQKVLGAVKPIVGAFVPDCDEKGEYRPKQCHGSTGHCWCVS |
| | | | | KDGKEIQGTRVAPGQSPPTCQDTVLSAETSCIKERQKVLGAVKP |
| | | | | ILGAFLPDCDEKGEYRPKQCHGSTGYCWCVSKDGKEIQGTRAA |
| | | | | PGQSPPTCQDTGEKLVLSAETSCIKERQKVLGAVKPIVGAFVPD |
| | | | | CDEKGEYRPKQCHGSTGHCWCVSKDGKEIQGTRVAPGQSPPT |
| | | | | CQDTVLSAETSCIKERQKVLGAVKPILGAFVPDCDEKGEYRPK |
| | | | | QCHGSTGYCWCVSKDGKEIQGTRAAPGQSPPTCQDTGEKLVLS |
| | | | | AETSCIKERQKVLGAVKPIVGAFVPDCDEKGEYRPKQCHGSTG |
| | | | | HCWCVSKDGKEIQGTRVAPGQSPPTCQDTDPVTCHYAVAVVK |
| | | | | KSSTLQFNQLKGKRSCHSAVGKTAGWIAPLYKLYKKNLLLWE |
| | | | | KPEEKSFEKAASEFFSVSCAPGAKEENLCKQCAGKEDKCKRSP |
| | | | | GELYYGDEGALRCLREDKGDVAFLEDIALSGQDLDNFELLCPD |
| | | | | NTKSPLSEQKHCHFGKVPSHAVVTRSTGDKSKDIIEYLLEAQKK |
| | | | | GCKLFSSTHGKDLMFEHTTTNVIALPSAMNTFLFLGPELFDAM |
| | | | | KTLHGTPLPSKNEVRWCTQNQNEKTKCDDWSSVSGGAIKCTEP |
| | | | | SSVQQCIEKILKHEADAVTLSAEHMYTALKCGLVPAVDEYHNK |
| | | | | DDFAPCRTLGDIYTDFGTPRAVALVKKSNKDITWNNLKGKKSC |
| | | | | HTGVGHMAGWVIPLSLISKQTNNCDLGSYFSQSCAPGSAIDSNL |
| | | | | CKLCIGDPQNTKAQTQCSPSDKEAYYDSAGAIRCLVEKGDVAF |
| | | | | LPHTAVFENTDGNNPALWAKDLKSTDFELLCPDGSRAPVTDYK |
| | | | | NCKLLSISSPSVVTREESVSDVVRIVLSQQSLFGRKGFEKDMFQ |
| | | | | MFSSSNGKNLLFSDGTQCLLEFDRIIGRDIMEDYFGKPFHTAVH |
| | | | | RDNQCLPTSELASACAFHHC* |
| 12 | BgSxph | Bufo | Asiatic | MKHSSSSAQKEVRMTPAFQVLLCLCLLGMSYAAPQARNVRW |
| | | gargarizans | toad | CVTSNAEEKKCNDLVNSCTVKEIVLICVKKSSTEDCMRAISAGE |
| | | | | ADAISLDSENIYKASLNPFDLKPIMTETYPSQKEIKVNQGPCQKE |
| | | | | RQRQRERGRPLLGAFEPKCDEKGNYQPKQCHGSTGYCWCVNE |
| | | | | EGKTIDGTKTPPGQKSVTCEDHQQSTPCLRHRQSVLGANKPQIG |
| | | | | AFVPDCDEKGNYSPKQCFGSTGYCWCVDEHGDEIEGVRAKQG |
| | | | | KVNITCEYTGGEKPCMKERRKSLSGGQPLPGAFMPDCDEKGN |
| | | | | YSPKQCHGSTGYCWCVNENGKEISGSRTPPGQQVPTCGASVLS |
| | | | | AETSCIKERQKVLGAATPILGAFVPDCDAKGDYRPKQCHGSTG |
| | | | | HCWCVSKDGKEIQGTRTAPGQTPPTCEIPGGEKPCMKERRKSL |
| | | | | SGGQPLPGAFMPDCDEKGNYSPKQCHGSTGYCWCVNENGKEI |
| | | | | SGSRTPPGQQVPTCGASVLSAETSCIKERQKVLGAEKPILGAFV |
| | | | | PDCDEKGDYRPKQCHSSTGHCWCVSKDGKEIQGTRTAPGQTPP |
| | | | | TCEIPGGEKPCMKERQKSLSGGQPLPGAFMPDCDEKGNYSPKQ |
| | | | | CHGSTGYCWCVNENGKEISGSRTPPGQQVPTCGASVLSADTSC |
| | | | | IKERQKVLGAEKPILGAFEPDCDEKGDYRPKQCHGSTGHCWCV |
| | | | | SKDGKEIQGTRTARGQSPPTCEIPVLSAETSCIKERQKVLGAATP |
| | | | | ILGAFVPDCDEKGDYRPKQCHGSTGHCWCVSKDGKEIQGTRA |
| | | | | APGQTPPTCEDRVLSAETSCIKERQKVLGAATPILGAFVPDCDE |
| | | | | KGEYRPKQCHGSTGHCWCVSKDGKEIQGTRAGPGQTPPTCED |
| | | | | TAETSCIKERQKVLGAAKPILGAFVPDCDEKGEYRPKQCHGST |
| | | | | GHCWCVSKDGKEIQGTRAAPRQSPPTCEIPVLSAETSCIKEQQK |
| | | | | VRAGKPILGAFVPDCDEKGDYRPKQCHGSTGHCWCVSKDGKE |
| | | | | IQGTRAACGQSPPTCEIPAETSCIKERQKVLGAEKPILGAFVPDC |
| | | | | DEKGEYRPKQCHGSTGHCWCVSKDGKEIQGTRAGPGQSPPTC |
| | | | | EDTVLSAETSCIKERQKVLGAATPILGAFVPDCDEKGDYRPKQC |
| | | | | HGSTGHCWCVSKDGKEIQGTRAGPGQSPPTCEIPAEKSCIKERQ |
| | | | | KVRSPRKPILGAFVPDCDEKGDYRPKQCHSSTGHCWCVSKDG |
| | | | | KEIQGTRAARGQSPPTCEDPDPVTCHYAVAVVKKSSTLQFNQL |
| | | | | KGKRSCHSAVGKTASWIAPLYKLYKKNLLLWEKPEEKSFEKA |
| | | | | ASEFFSVSCAPGAKEENLCKQCAGKEEKCKRSPGEPYYGDEGA |
| | | | | LRCLRDDKGDVAFVEHTALSGQDLDNFELLCPDNTRSPLSEHK |
| | | | | HCHFGKVPRHAVVTKSTGDKSKDIIEYLLEAQKKGCKLFSSTH |
| | | | | GKDLIFEHTTKNLIALPSAMNTFLFLGPELFDAMKTLHGAPLPS |
| | | | | KNEVRWCTQNKKEKTKCDDWSSVSGGAIKCTEPSSVQQCIEKI |
| | | | | LKHEADAVTLSAEHMYTALKCGLVPAIDEYHNKDDLAPCKTL |
| | | | | GDIYTDFGTPRAVALVKKSDKGITWNNLKGKKSCHTGVGHMA |
| | | | | GWVIPLSLISKQTNNCDMGSYFSQSCAPGSAIDSNLCKLCIGDP |
| | | | | QNTKAQTKCSPSDKEAYFGSAGAIRCLVEKGDVAFLPHTAVIE |
| | | | | NTDGNNPALWAKDLKSTDFELLCPDGSRAPVTDYKNCKLLSIS |
| | | | | SPSVVTREESVSDVVRIVLNQQSLFGQKGFEKDIFQMFSSSNGK |
| | | | | NLLFSDGTQCLLEFDRIIGRDIMEDYFGKRFHTAVHRDNQCLPT |
| | | | | SEFASACAFHHC* |
| 13 | EpSxph | Engystomops | Tungara | MTAISHGEADAISLHGENIYKASFKPFNLIPIITESYHYQKGGRPL |
| | | pustulosus | frog | LGAFVPKCDEKGNYSPTQCHGGTGYCWCVTAEGKEIDGTKKP |
| | | | | PGESPTCGEETKKTPCLNKREKALSGGQPLLGAFAPDCDEEGN |
| | | | | FKPRQCYGSTGYCWCVDDNGVEISATRTPPGEKPPTCGATDPV |
| | | | | TCHYAVAVVKKSSTFQFNQLKGKRSCHSAVGESAGWVAPMS |
| | | | | ALLDKKFLLWEGPAKKSFEKAASEFFSASCAPGAKEENLCKQC |
| | | | | AGQQDKCKQSPGEPYYGDEGAFRCLREDKGDVAFVEHTVLSG |
| | | | | QYSDNYELLCPDKTRKPLSQYEHCHFGKVPRPAVVTRHTGRKT |
| | | | | KDITDYLLEAQKKECKLFSSTHGKNPPDTTTTLTSLPLAMDTFL |
| | | | | FLGPELFNAMKRMHGEPLPSNKEVRWCPQSSDEKKKCDDWSA |
| | | | | VSGGAIKCTEPFTTLQCIEKVLKGEAEAVTLNVENMYTALKCG |
| | | | | LVPIVEEYHNKDDFNPCQSRGAQIKDFGTVKAVAVVKKRDKDI |
| | | | | NWNNLKGKKSCHTGVGHLAGWVVPVSLINKQSRTCDVESYFN |
| | | | | QSCAPGSTTKSNLCKLCIGDPQNTKAKTKCSPNDKEAYYGNEG |
| | | | | AIRCLAEKGDVAFVPHTAVFENTDGKNPALWAKDLKSTDFELL |
| | | | | CPDGSRAPVSNYKNCKLAGIPPRAVVTRKDSASDVTKIIVNQQS |
| | | | | LFGRKGFQKDIFQMFSSSNGQNLLFSDNTQCLIEFDRMIEKDIM |
| | | | | EDYFGKTYYTAVHSDNQCFPPSALASACSFHHC* |
| 14 | EcSxph | Eleuthero- | Puerto | MTPAFQLLLCLCLLGLSYTAPQGRNVRWCVTSDIEDKKCNDLA |
| | | dactylus | Rican | NSCTVNEIHLKCVKKSSTLDCVKTISDGEADAISLDGEDIYKAS |
| | | coqui | coqui | LNPFNLKPIMVESHYHRAKNLPCLKERQEALGGGHPLPGAFVP |
| | | | | NCDEKGNYNPKQCHGSTGYCWCVNKNGQKINGTETPPGQPSP |
| | | | | TCEEEKSTPCLKHRQRLLGGNKPKIGAFVPKCDEKGDYIPKQC |
| | | | | HGSTGQCWCVNTDGEEIAGTRTGPGKAPPTCEDAEPVTCHYA |
| | | | | VAVVKKSSTLQFDQLKGKRSCHSAVGTAAGWAAPLNALMKR |
| | | | | NLLVWKGPKEKTIEKAASEFFLSSCAPGAKETNLCKQCAGKEA |
| | | | | NCKHSSEEPYYGDEGAFKCLRDDKGDVAFVENSVLSEEHTDSF |
| | | | | ELLCPDNTRKQLSQYKDCNFGKVPRHAVVTRSSGDKIKDITEY |
| | | | | LLGAQKKECELFSSTHGKRFLFEDTTTNLIALPSAMDTFLFLGPE |
| | | | | LFDAMKALHGEHPPSKNEVRWCTLSEKEKLKCDDWSSVSGGA |
| | | | | IKCTEPSLAEECIEKVLKGEADVVSLSKKHMYAALTCGLIP AFE |
| | | | | EYHNKDDFGPCQTPGTQYTDFGMTSNVALVKKKDQDITWNNL |
| | | | | KGKKSCHPEVDDSAGWVDPLILIKKQTKSCDLGSFFSKSCAPGS |
| | | | | AIDSNLCELCIGDSQNSKANTKCSRSDKEAYYGNDGAIRQKGD |
| | | | | VAFVPHFAVSDNTDGKNPALWAKDLKSSDFELLCPDGSRAPVS |
| | | | | DHKKCKLGGASIQTVVCREDRVSDVVRIIQNQQSLYGRKGFQK |
| | | | | DIFQIFSSRHGRNILFHDDTQCLVDFDRVKEIDIMYDFFGKEHY |
| | | | | DSIDFCLLIPSLIRRLGNRDLVIEMSLLVVFIPPS* |
| 15 | RtSxph | Rana | European | MAPTFKAALFFTIISLSFAAPNAKQVRWCAISDLEQKKCNDLVG |
| | | temporaria | common | SCNVQDITLVCVLRTSTEDCLTAIKDGQADAMFLDSGDVYEAS |
| | | | frog, | KDPYNLKPIIAEPYSSHKDLQKCLKEREQALAKKMIGHHIPQCD |
| | | | European | EKGNYQPQQCHGSTGQCWCVNGMGEKISGTNTPPGQTRATCE |
| | | | common | RHELPKCLKERQVALGGDEIVLGRFVPQCDEKGNYEPQQFHGS |
| | | | brown frog, | TGYSWCVNEIGEEIAGTKTPPGKIPVTCEKHDLVTTCHYAVAM |
| | | | European | VKKSSTFQFNQLKGKRSCHSGVSKTDGWKALVTVLVEKKLLS |
| | | | grass frog | WDGPAKESIQQAMSKFFSVSCIPGATQTNLCKQCKGEEGKNCK |
| | | | | NSHDEPYYGNHGASRCLKEDKGDVAFLRSTALSDEDSEAYELL |
| | | | | CPDNTRKPLNKYKECNLGTVPVGAVVTRKIGDKTVDINNFLME |
| | | | | AQKKQCKLFSSAHGKDLMFDDSTLQLALLSSGVDAFLYLGVK |
| | | | | LFNTMKALTEDAHLSSKNKVRWCTINKLEKMKCDDWSAVSG |
| | | | | GAIECTEASCPKCCIKQILKGEADAVKLEVQYMYEALTCGLLP |
| | | | | AVEEYHNKDDFGPCKTPGSPYTDFGSPRAVALVKKSNKDINW |
| | | | | NNIKGKKACLTGVGDIAGWVIPVSLIRRQNDNCDIDSFFGESCA |
| | | | | PGSDTKSNLCKLCIGDPKNSVANTKCSLGDNEAYHGNKGAFRC |
| | | | | LVEKGDVAFVPHTVVFENTDGKNPAEWAKKLKSEDFELLCLD |
| | | | | GSRSPVSNYKSCKLSGIPPPAIVTREQSVTDVVRIVTNQQSLYGR |
| | | | | KGFEKDIFQLFSSNKGKNLLFNDNTQCLIEFDRQPKDIMEDYFG |
| | | | | KSYYTTVYGASRSAMSSELISACTVKHC* |

*End of sequence
^{†}Sequences that we obtained directly.

TABLE 2
RcSxph and NpSxph Mutations

| | NpSxph Mutations in Nanorana |
|---|---|
| RcSxph Mutations in Rana | parkeri (High Himalaya frog, |
| (Lithobates) catesbeianus | Xizang Plateau frog, Parker's |
| (American bullfrog, Bullfrog) | slow frog, mountain slow frog) |
| I782A/Y558A | I559Y |
| Y558I | |
| Y558A | |
| I782A | |
| D785N | |
| K789A | |
| T563A | |
| Y558F | |
| Q787E | |
| Y795A | |
| Q787A | |
| F784Y | |
| I782F | |
| F561A | |
| D785A | |
| F784L | |
| P727A | |
| E540D | |
| F784A | |
| D794E | |
| F784C | |
| D794N | |
| F784S | |
| E540Q | |
| D794A | |
| E540A | |

The present disclosure provides pharmaceutical compositions comprising proteins or amino acid sequences comprising at least 80% o sequence identity to SEQ ID NO: 1 and comprising at least one of the following amino acid substitutions in SEQ ID NO: 1: an alanine for the isoleucine at position 782; an alanine for the tyrosine at position 558; an isoleucine for the tyrosine at position 558; an asparagine for the aspartic acid at position 785; an alanine for the lysine at position 789; an alanine for the threonine at position 563; a phenylalanine for a tyrosine at position 558; a glutamic acid for the glutamine at position 787; an alanine for a tyrosine at position 795; an alanine for the glutamine at position 787; a tyrosine for the phenylalanine at position 784; a phenylalanine for the isoleucine at position 782; an alanine for the phenylalanine at position 561; an alanine for the aspartic acid at position 785; a leucine for the phenylalanine at position 784; an alanine for the proline at position 727; an aspartic acid for the glutamic acid at position 540; an alanine for the phenylalanine at position 784; a glutamic acid for the aspartic acid at position 794; a cysteine for the phenylalanine at position 784; a asparagine for an aspartic acid at position 794; a serine for the phenylalanine at position 784; a glutamine for the glutamic acid at position 540; an alanine for the aspartic acid at position 794; an alanine for the glutamic acid at position 540.

The present disclosure provides pharmaceutical compositions comprising proteins or amino acid sequences comprising at least 80% sequence identity to SEQ ID NO:1 and comprises a tyrosine for the isoleucine at position 559. In some embodiments, the protein comprises at least 80% sequence identity to SEQ ID NO:1, and comprises at least one of the amino acid substitutions in SEQ ID NO:1. In some embodiments, the protein comprises at least 90% sequence identity to SEQ ID NO:1, and comprises at least one of the amino acid substitutions in SEQ ID NO:1. In some embodiments, the protein comprises at least 95% sequence identity to SEQ ID NO:1, and comprises at least one of the amino acid substitutions in SEQ ID NO:1. In some embodiments, the protein comprises at least 99% sequence identity to SEQ ID NO:1, and comprises at least one of the amino acid substitutions in SEQ ID NO:1. In some embodiments, the protein comprises at least 80% sequence identity to SEQ ID NO:2, and comprises at least one of the amino acid substitutions in SEQ ID NO:2. In some embodiments, the protein comprises at least 90% sequence identity to SEQ ID NO:2, and comprises at least one of the amino acid substitutions in SEQ ID NO:2. In some embodiments, the protein comprises at least 95% sequence identity to SEQ ID NO:2, and comprises at least one of the amino acid substitutions in SEQ ID NO:2. In some embodiments, the protein comprises at least 99% sequence identity to SEQ ID NO:2, and comprises at least one of the amino acid substitutions in SEQ ID NO:2.

In some embodiments, the present disclosure provides pharmaceutical compositions comprising an amino acid sequence comprising at least 80% sequence identity to SEQ ID NO:1, and comprises more than one of the amino acid substitutions in SEQ ID NO:1. In some embodiments, the protein or amino acid sequence comprises at least 90% sequence identity to SEQ ID NO:1, and comprises more than one of the amino acid substitutions in SEQ ID NO:1. In some embodiments, the protein comprises at least 95% sequence identity to SEQ ID NO:1, and comprises more than one of the amino acid substitutions in SEQ ID NO:1. In some embodiments, the protein comprises at least 96% sequence identity to SEQ ID NO:1, and comprises more than one of the amino acid substitutions in SEQ ID NO:1. In some embodiments, the protein comprises at least 97% sequence identity to SEQ ID NO:1, and comprises more than one of the amino acid substitutions in SEQ ID NO:1. In some embodiments, the protein comprises at least 98% sequence identity to SEQ ID NO:1, and comprises more than one of the amino acid substitutions in SEQ ID NO:1. In some embodiments, the protein comprises at least 99% sequence identity to SEQ ID NO:1, and comprises more than one of the amino acid substitutions in SEQ ID NO:1.

In some embodiments, the protein or amino acid sequence comprises at least 80% sequence identity to SEQ ID NO:2, and comprises more than one of the amino acid substitutions in SEQ ID NO:2. In some embodiments, the protein comprises at least 90% sequence identity to SEQ ID NO:2, and comprises more than one of the amino acid substitutions in SEQ ID NO:2. In some embodiments, the protein comprises at least 95% sequence identity to SEQ ID NO:2, and comprises more than one of the amino acid substitutions in SEQ ID NO:2. In some embodiments, the protein comprises at least 96% sequence identity to SEQ ID NO:2, and comprises more than one of the amino acid substitutions in SEQ ID NO:2. In some embodiments, the protein comprises at least 97% sequence identity to SEQ ID NO:2, and comprises more than one of the amino acid substitutions in SEQ ID NO:2. In some embodiments, the protein comprises at least 98% sequence identity to SEQ ID NO:2, and comprises more than one of the amino acid substitutions in SEQ ID NO:2. In some embodiments, the protein comprises at least 99% sequence identity to SEQ ID NO:2, and comprises more than one of the amino acid substitutions in SEQ ID NO:2.

In some embodiments, the protein or amino acid sequence comprises SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15. In some embodiments, the protein or amino acid sequence comprises at least about 70% or 80% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO: 11, SEQ ID NO:12, SEQ ID NO:13, SEQ ID NO:14, or SEQ ID NO:15 but is free of SEQ ID NO:1, SEQ ID NO:2, SEQ ID NO:3, SEQ ID NO:4, SEQ ID NO:5, SEQ ID NO:6, SEQ ID NO:7, SEQ ID NO:9, SEQ ID NO:10, SEQ ID NO:11, SEQ ID NO:12, SEQ ID NO:13, SEQ ID NO:14, SEQ ID NO:15, respectively.

The present invention also provides nucleic acids encoding any of the proteins described above. In some embodiments, the nucleic acid comprises. The present invention also provides vectors comprising any of the nucleic acids described above encoding any of the proteins described above. In some embodiments, the vector is a plasmid. In some embodiments, the vector is a retrovirus.

In some embodiments, the protein or amino acid sequence comprises at least 80% sequence identity to SEQ ID NO:1, and comprises more than one of the amino acid substitutions in SEQ ID NO:1. In some embodiments, the mutation is a substitution or deletion mutation 540, 558; 561, 563, 727, 782, 784, 785, 787, 789, 794 and/or 795.

The present disclosure relates to a pharmaceutical composition comprising: (i) a therapeutically effective amount of SEQ ID NO:1 or an amino acid sequence comprising about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1; and (ii) a pharmaceutically acceptable carrier. In some embodiments, the ratio of amino acid sequence to carrier is 1:1 or 1:2 or 2:1. The present disclosure relates to a pharmaceutical composition comprising: (i) a therapeutically effective amount an amino acid sequence comprising about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1; and (ii) a pharmaceutically acceptable carrier, wherein the amino acid sequence comprises one or a combination of substitutions or deletion mutants at position: 540, 558; 561, 563, 727, 782, 784, 785, 787, 789, 794 and/or 795.

The present disclosure relates to a pharmaceutical composition comprising: (i) a first amino acid sequence that comprises at least about 70% sequence identity to X₁X₂X₃X₄X₅X₆X₇X₈X₉X₁₀X₁₁X₁₂X₁₃X₁₄; and (ii) a pharmaceutically acceptable carrier; wherein the first amino acid sequence comprises at least about 70% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V. In some embodiments, the amino acid sequence comprises at least 90% sequence identity to X₁X₂X₃X₄X₅X₆X₇X₈X₉X₁₀X₁₁X₁₂X₁₃X₁₄; wherein X₁=I, V, F, L; X₂=any amino acid; X₃=F; X₄=D; X₅=any amino acid; X₆=M, Q, I; X₇=any amino acid; X₈=R, K; X₉=any amino acid; X₁₀=any amino acid; X₁₁=any amino acid; X₁₂=any amino acid; X₁₃=D; X₁₄=Y, V; or a pharmaceutically acceptable salt thereof, and a pharmaceutically acceptable carrier.

In some embodiments, the first amino acid sequence comprises at least about 75% sequence identity to an amino acid comprising contiguous amino acids with Formula: X_{B}−[from about 53 to about 56 amino acids]−[[X₁X₂X₃X₄X₅X₆X₇X₈X₉X₁₀X₁₁X₁₂X₁₃X₁₄]; wherein X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid sequence comprises at least about 75% sequence identity to an amino acid with contiguous amino acids of Formula III: [X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein X₁ₐ=Y; X₂ₐ=any amino acid; X₃ₐ=an amino acid; X₄ₐ=F; X₅ₐ=any amino acid; X₆ₐ=S or G; X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V. In some embodiments, the first amino acid sequence comprises at least about 75% sequence identity to an amino acid with contiguous amino acids of Formula IV: [X_{A}]−[from about 15 to about 20 amino acids]−[X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein X_{A}=D or E; X₁ₐ=Y; X₂ₐ=any amino acid; X₃ₐ=an amino acid; X₄ₐ=F; X₅ₐ=any amino acid; X₆ₐ=S or G; X_{B}=N or P; X₁=I, V, F, or L; X₂=any amino acid; X₃=F; X₄=D; X₅=any amino acid; X₆=M, Q, or I; X₇=any amino acid; X₈=R or K; X₉=any amino acid; X₁₀=any amino acid; X₁₁=any amino acid; X₁₂=any amino acid; X₁₃=D; X₁₄=Y or V.

In some embodiments, the first amino acid is chosen from an amino acid sequence comprising at least about 75% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14 or SEQ ID NO:15 or a functional fragment thereof. In some embodiments, the first amino acid is a functional fragment comprising at least about 75% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12. In some embodiments, the first amino acid is chosen from an amino acid sequence comprising a substitution mutation at amino acid number chosen from: 540, 558, 559, 561, 563, 727, 782, 784, 785, 787, 789, 794 and/or 795, in relation to such amino acid numbers identified in any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO:15, or SEQ ID NO:16.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In further embodiments, the pharmaceutical composition is administered to a mammal. In still further embodiments, the mammal is a human. In an even further embodiment, the human is a patient.

In further embodiments, the pharmaceutical composition is administered following identification of the mammal in need of treatment of a disorder associated with NO neuron activity. In still further embodiments, the mammal has been diagnosed with a need for treatment of a disorder associated with PSP prior to the administering step.

In further embodiments, the pharmaceutical composition is administered following identification of the mammal in need of treatment of a PSP. In still further embodiments, the mammal has been diagnosed with a need for treatment of a PSP prior to the administering step.

In various embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1, 3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcelluslose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example. dimethyldialkylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, PA, Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the treatment (e.g., prophylactic or therapeutic) of a PSP. The method also includes the administration of a therapeutically effect amount of the compound for the treatment of patient having a predisposition for being afflicted with a PSP. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal, the body weight of the animal, as well as the severity and stage of the disorder.

The total amount of the compound of the present disclosure administered in a typical treatment is preferably from about 1 mg/kg to about 100 mg/kg of body weight for mice, and from about 10 mg/kg to about 50 mg/kg of body weight, and from about 20 mg/kg to about 40 mg/kg of body weight for humans per daily dose. This total amount is typically, but not necessarily, administered as a series of smaller doses over a period of about one time per day to about three times per day for about 24 months, and over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In certain some embodiments, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

A compound described herein can be administered alone or can be coadministered with an additional therapeutic agent. Thus, the preparations can also be combined, when desired, with other active substances (e.g., to reduce metabolic degradation). Additional therapeutic agents include, but are not limited to, other active agents known to be useful in treating a PSP as further described herein.

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, 1.2% saline, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, tosylate, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A.R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present disclosure is in the form of a liquid wherein the active agent is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of the compounds described herein in accordance with the present disclosure include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof, phenoxyethanol; chlorophenoxyethanol; phenoxypropanol; chlorobutanol; chlorocresol; phenylethyl alcohol; disodium EDTA; and sorbic acid and salts thereof.

It is understood that the disclosed compositions can be prepared from the disclosed compounds. It is also understood that the disclosed compositions can be employed in the disclosed methods of using.

### D. Kits

In some embodiments, disclosed are kits comprising a disclosed amino acid sequence herein, a functional fragment thereof, or a pharmaceutically acceptable salt thereof, and one or more selected from: (a) instructions for treating a PSP; and (b) instructions for administering the amino acid in connection with treating PSP.

In further embodiments, the kit comprises the agent known for the treatment of a PSP. Examples of agents known for the treatment of PSP include, but are not limited to, antibodies specific for a disclosed toxin.

In further embodiments, the compound and the at least one agent are co-formulated. In further embodiments, the compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the disclosure concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the disclosure to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### EXAMPLES

### Example 1: Definition of a Saxitoxin (STX) Binding Code Enables Discovery and Characterization of the Anuran Saxiphilin Family

To characterize RcSxph:STX interactions in detail, we developed a suite of assays comprising thermofluor (TF) measurements of ligand-induced changes in RcSxph stability, fluorescence polarization (FP) binding to a fluorescein-labeled STX, and isothermal titration calorimetry (ITC). We paired these assays with a scanning mutagenesis strategy (Clackson and Wells, Wells 1991) to dissect the energetic contributions of RcSxph STX binding pocket residues. These studies show that the core RcSxph STX recognition code comprises two ‘hot spot’ triads. One engages the STX tricyclic bis-guanidinium core through a pair of carboxylate groups and a cation-p interaction (Infield et al.) in a manner that underscores the convergent STX recognition strategies shared by RcSxph and Na_{V}s (Infield et al., Thomas-Tran and Du Bois, Heinemann et al., Satin et al., Leffler et al., Sivilotti et al.). The second triad largely interacts with the C13 carbamate group of STX and is the site of interactions that can enhance STX binding affinity and the ability of RcSxph to act as a ‘toxin sponge’ that can reverse the effects of STX inhibition of Na_{V}s (Mahar et al., Abderemaine-Ali et al.).

Although Sxph-like STX binding activity has been reported in extracts from diverse organisms including arthropods (Llewellyn et al.), amphibians (Doyle et al., Llewellyn et al., Tanaka et al.), fish (Llewellyn et al.), and reptiles (Llewellyn et al.), the molecular origins of this activity have remained obscure. Definition of the RcSxph STX recognition code enabled identification of ten new Sxphs from diverse frogs and toads. This substantial enlargement of the Sxph family beyond RcSxph and the previously identified High Himalaya frog (Nanorana parkeri) Sxph (NpSxph) (Yen et al.) reveals a varied STX binding pocket surrounding a conserved core of ‘hot spot’ positions and dramatic differences in the number of thyroglobulin (Thy1) domains inserted into the modified transferrin fold upon which Sxph is built. Biochemical characterization of NpSxph, Oophaga sylvatica Sxph (OsSxph) (Caty et al.), Mantella aurantica Sxph (MaSxph), and Ranitomeya imitator Sxph (RiSxph), together with structural determination of NpSxph shows that the different Sxphs retain the capacity for high affinity STX binding and that binding site preorganization (Yen et al.) is a critical factor for STX binding. Together, these studies establish a STX molecular recognition code that provides a framework for understanding how diverse STX binding proteins engage the toxin and its congeners and uncover that Sxph family members are abundantly found in the most varied and widespread group of amphibians, the Anurans.

### 1. Results

### Establishment of a Suite of Assays to Probe RcSxph Toxin Binding Properties

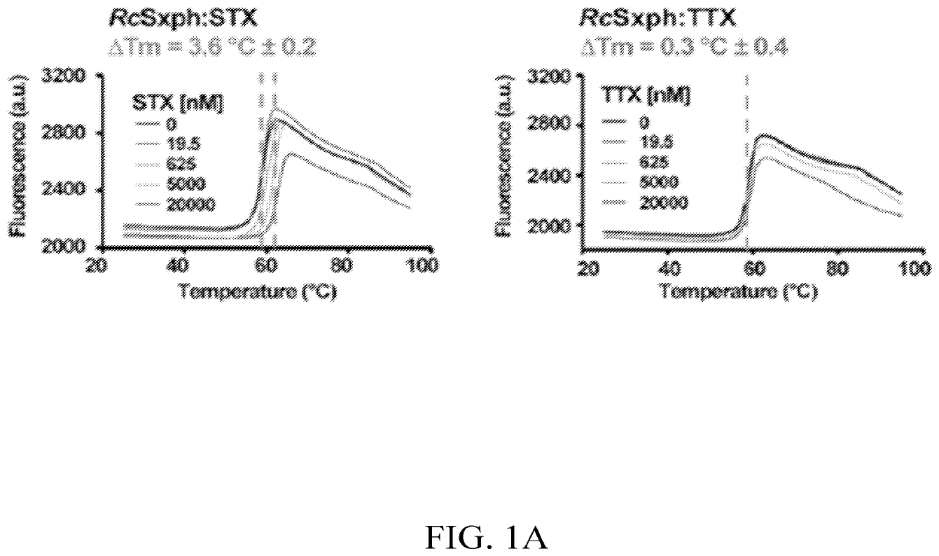

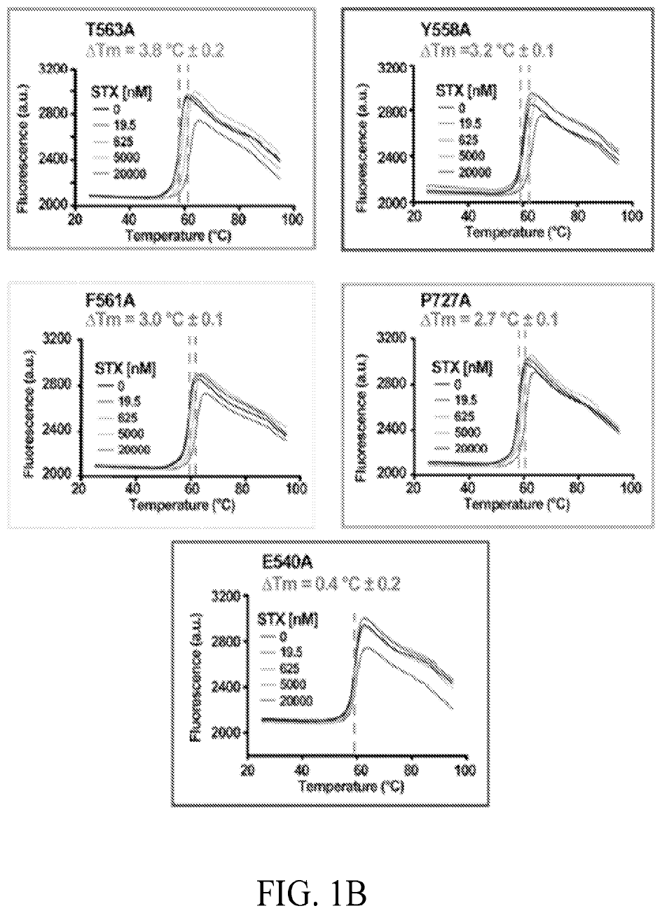

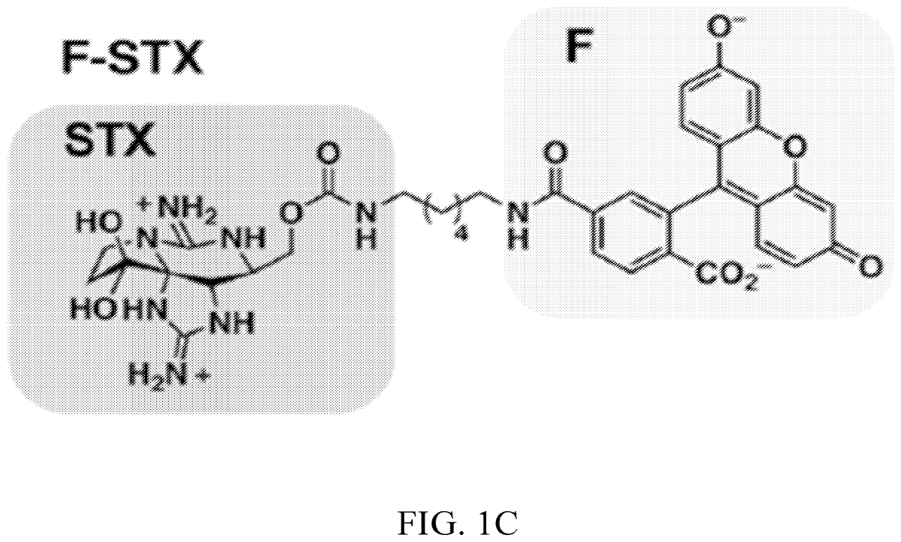

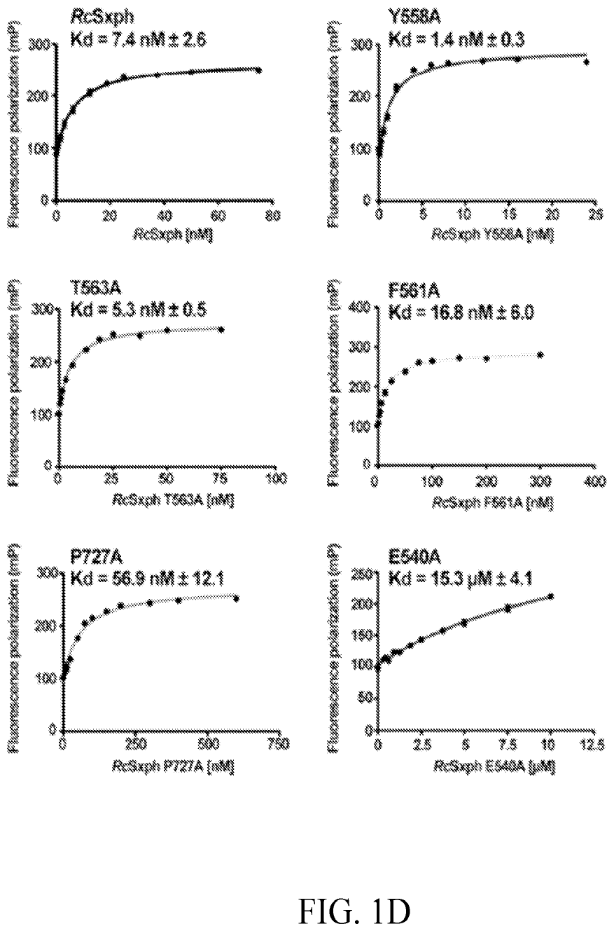

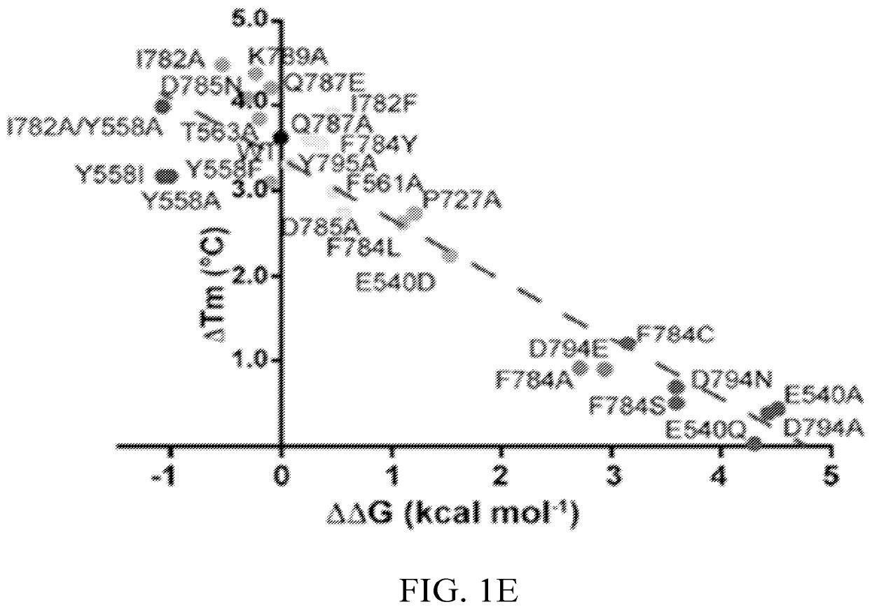

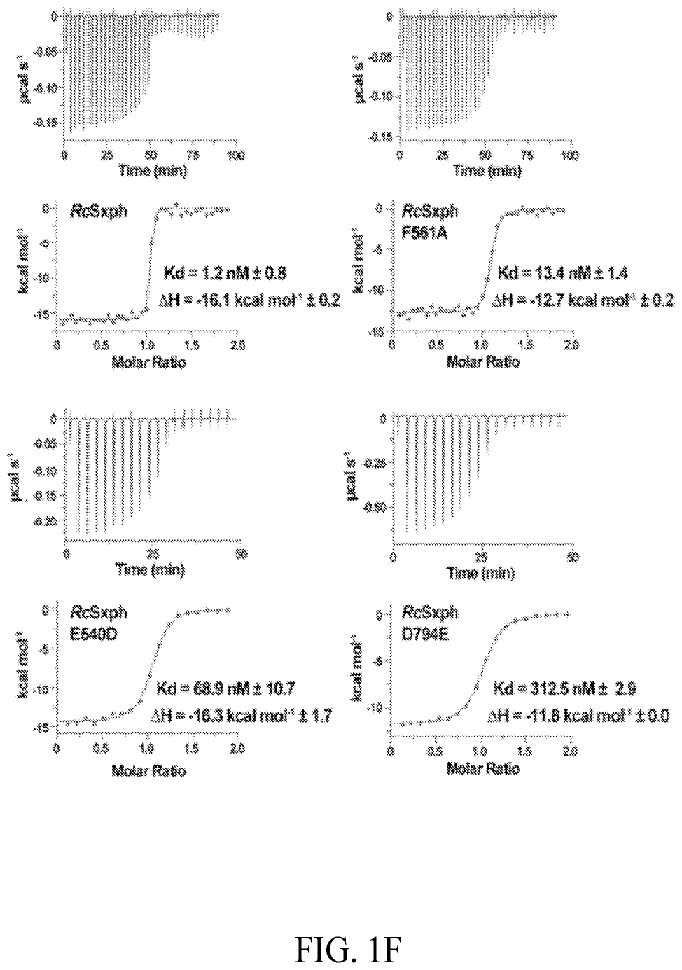

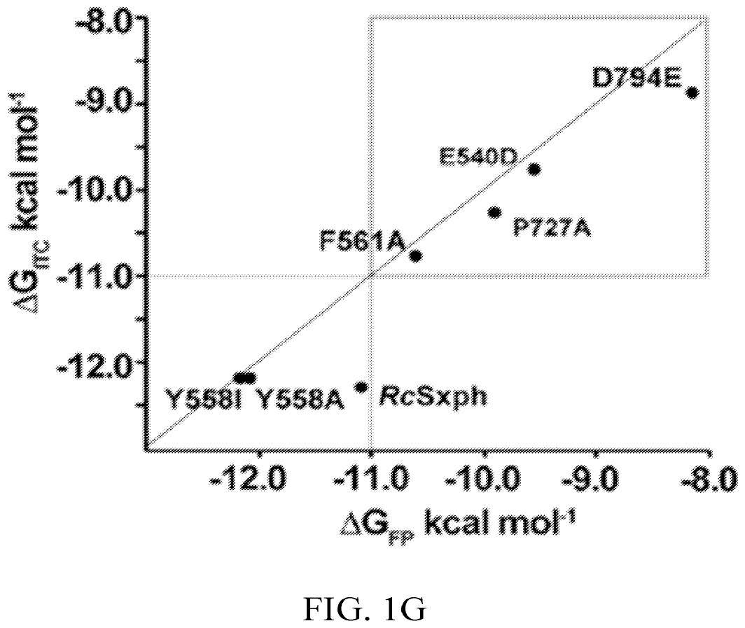

To investigate the molecular details of the high-affinity RcSxph:STX interaction, we developed three assays to assess the effects of STX binding site mutations. A key criterion was to create assays that could be performed in parallel on many RcSxph mutants using minimal amounts of purified protein and toxin. To this end, we first tested whether we could detect STX binding using a thermofluor (TF) assay (Huynh and Partch, Niesen et al.) in which STX binding would manifest as concentration-dependent change in the apparent RcSxph melting temperature (Tm). Addition of STX, but not the related guanidinium toxin, tetrodotoxin (TTX), over a 0-20 μM range to samples containing 1.1 μM RcSxph caused concentration-dependent shifts in the RcSxph melting cure (FIG. 1A)(DTm=3.6° C.±0.2 versus 0.3° C.±0.4 for STX and TTX, respectively). These differential effects of STX and TTX are in line with the ability of RcSxph to bind STX (Mahar et al., Llewellyn et al., Llewellyn and Moczydlowski) but not TTX (Mahar et al., Abderemane-Ali et al.) and indicate that DTm is a consequence of the RcSxph:STX interaction.

To investigate the contributions of residues that comprise the STX binding site, we coupled the TF assay with alanine scanning (Clackson and Wells) as well as deeper mutagenesis studies targeting the eight residues that directly contact STX (Glu540, Phe561, Thr563, Tyr558, Pro727, Phe784, Asp785, Asp794) (Yen et al.) and four second shell sites that support these residues (Tyr795, Ile782, Gln787, and Lys789) (FIGS. 1A-B and FIG. S1A). Measurement of the STX-induced DTm changes for these twelve, purified RcSxph alanine mutants revealed DTm changes spread over a ˜4° C. range that included DTm increases relative to wild-type (e.g. I782A and D785N) as well as those that caused complete loss of the thermal shift (e.g. E540A and D794A). All mutations had minimal effects on protein stability (FIG. S1B) and there was no evident correlation between Tm and DTm (FIG. S1C). Hence, the varied DTms indicate that each of the twelve positions contribute differently to STX binding.

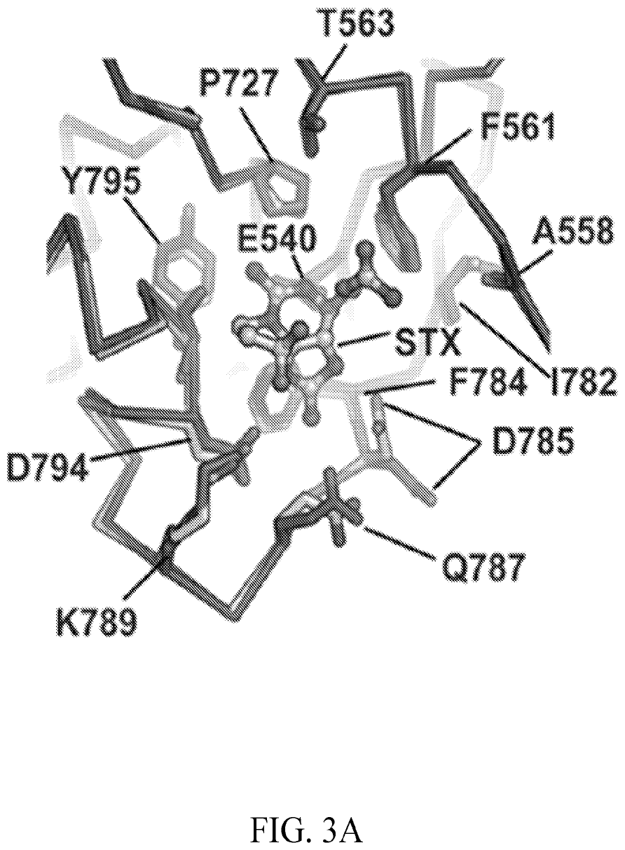

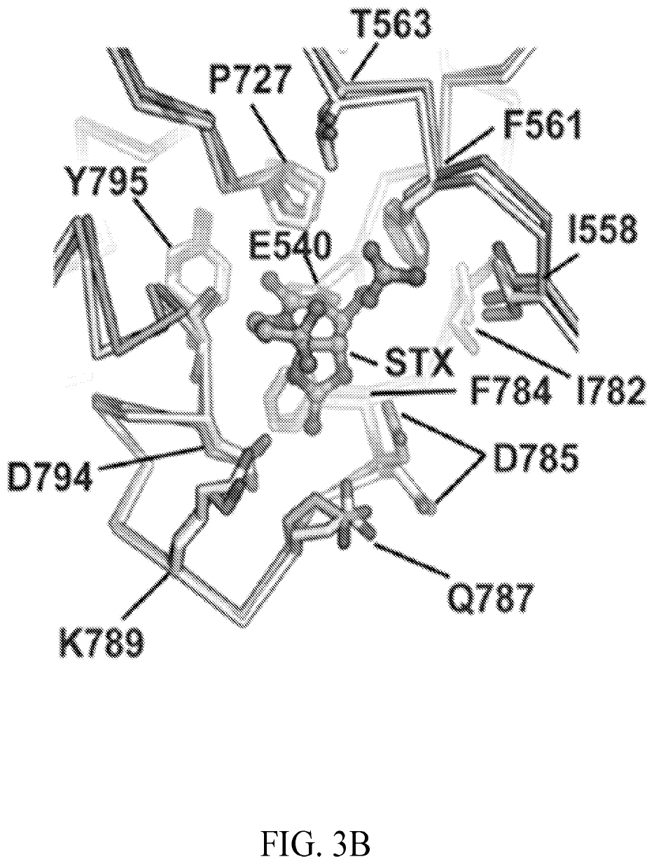

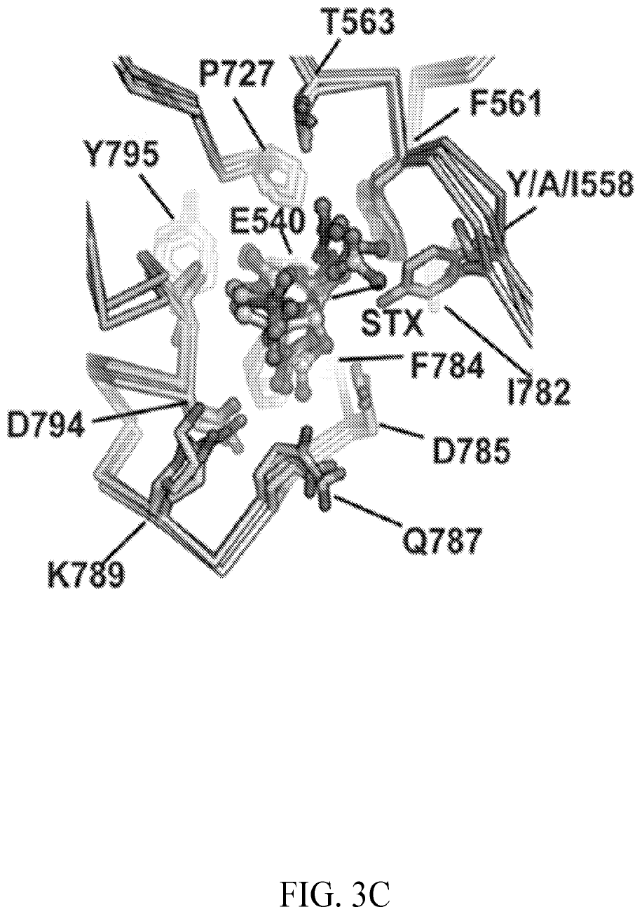

Because DTm interpretation can be complex, especially in the case of a multidomain protein such as RcSxph, and may not necessarily indicate changes in ligand affinity (Huynh and Partch, Niesen et al.), we developed a second assay to measure the effects of mutations on RcSxph binding affinity. We synthesized a fluorescein-labeled STX derivative (F-STX) by functionalization of the pendant carbamate group with a 6-carbon linker and fluorescein (Andresen and DuBois, Ondrus et al.) (FIGS. 1C and FIG. S2) and established a fluorescence polarization (FP) assay (Huang and Aulabaugh, Zhang et al.) to measure toxin binding. FP measurements revealed a high-affinity interaction between F-STX and RcSxph (Kd=7.4 nM±2.6) that closely agrees with prior radioligand assay measurements of RcSxph affinity for STX (˜1 nM) (Llewellyn and Moczydlowski). The similarity between the F-STX and STX Kd values is consistent with the expectation from the RcSxph:STX structure that STX carbamate derivatization should have a minimal effect on binding, as this element residues on the solvent exposed side of the STX binding pocket (Yen et al.). To investigate the F-STX interaction further, we soaked RcSxph crystals with F-STX and determined the structure of the RcSxph:F-STX complex at 2.65 Å resolution by X-ray crystallography (FIG. 3A, Table S1). Inspection of the STX binding pocket revealed clear electron density for the F-STX bis-guanidinium core as well as weaker density that we could assign to the fluorescein heterocycle (FIG. S3A), although the high B-factors of the linker and fluorescein indicate that these moieties are highly mobile (FIG. S3B). Structural comparison with the RcSxph:STX complex (Yen et al.) showed no changes in the core STX binding pose or STX binding pocket residues (RMSD_{Ca}=0.279 Å) (FIG. S3C). Together, these data demonstrate that both F-STX and STX bind to Sxph in the same manner and indicate that there are no substantial interactions with the fluorescein label.

FP measurement of the RcSxph alanine scan mutants uncovered binding affinity changes spanning a ˜13,000 fold range that correspond to free energy perturbations (DDG) of up to ˜5.60 kcal mol⁻¹ (FIG. 1D and FIG. S4, Table 1). The effects were diverse, encompassing enhanced affinity changes (Y558A Kd=1.2 nM±0.3) and large disruptions (E540A Kd=15.3 μM±4.1). As indicated by the TF data, each STX binding pocket residue contributes differently to STX recognition energetics. Comparison of the TF DTm and FP DDG values shows a strong correlation between the two measurements (FIG. 1E) and indicates that the changes in unfolding free energies caused by protein mutation and changes in STX binding affinity do not incur large heat capacity or entropy changes relative to the wild-type protein (Becktel and Schellman, Arrigoni and Minor). Hence, DTm values provide an accurate estimate of the STX binding affinity differences.

TABLE 1
RcSxph STX binding pocket mutant binding parameters

| | | | ΔΔG (kcal | |
|---|---|---|---|---|
| Class | Construct | Kd (nM) | mol⁻¹) | n |
| Enhanced binding | I782A/ | 1.2 ± 0.2 | −1.07 | 6 |
| ΔΔG < 1 kcal | Y558A | | | |
| mol⁻¹ | Y558I | 1.2 ± 0.2 | −1.07 | 6 |
| | Y558A | 1.4 ± 0.3 | −1.00 | 6 |
| Mild enhancement | I782A | 3.0 ± 0.8 | −0.53 | 4 |
| 1 ≤ ΔΔG ≤ 0 | D785N | 4.4 ± 0.6 | −0.30 | 4 |
| kcal mol⁻¹ | K789A | 5.1 ± 1.7 | −0.22 | 4 |
| | T563A | 5.3 ± 0.5 | −0.20 | 6 |
| | Y558F | 6.3 ± 2.3 | −0.10 | 4 |
| | Q787E | 6.4 ± 1.6 | −0.09 | 4 |
| | RcSxph | 7.4 ± 2.6 | 0 | 10 |
| Mild disruption | Y795A | 8.4 ± 2.1 | 0.08 | 4 |
| 0 ≥ ΔΔG ≥ 1 kcal | Q787A | 11.3 ± 1.1 | 0.25 | 4 |
| mol⁻¹ | F784Y | 13.8 ± 1.0 | 0.37 | 4 |
| | I782F | 16.1 ± 4.1 | 0.46 | 4 |
| | F561A | 16.8 ± 6.0 | 0.48 | 4 |
| | D785A | 19.5 ± 2.5 | 0.57 | 4 |
| Disruption | F784L | 48.0 ± 6.8 | 1.11 | 4 |
| 1 ≥ ΔΔG ≥ 2 kcal | P727A | 56.9 ± 12.1 | 1.21 | 4 |
| mol⁻¹ | E540D | 99.9 ± 25.1 | 1.54 | 4 |
| Strong disruption | F784A | 725.1 ± 108.7 | 2.71 | 4 |
| 2 ≥ ΔΔG ≥ 3 kcal | D794E | 1074.1 ± 69.3 | 2.94 | 4 |
| mol⁻¹ | | | | |
| Very strong | F784C | 1510.5 ± 346.1 | 3.15 | 4 |
| disruption | D794N | 3228 ± 397 | 3.60 | 4 |
| ΔΔG ≥ 3 | F784S | 3240 ± 508 | 3.60 | 4 |
| | E540Q | 10640 ± 1325 | 4.30 | 4 |
| | D794A | 13172 ± 6871 | 4.43 | 4 |
| | E540A | 15294 ± 4134 | 4.52 | 10 |

Kd, dissociation constant; n, number of observations
ΔΔG = RT In (Kd_{Sxph mutant}/Kd_{Sxph}); T = 298K
Errors for measurements are S.D.

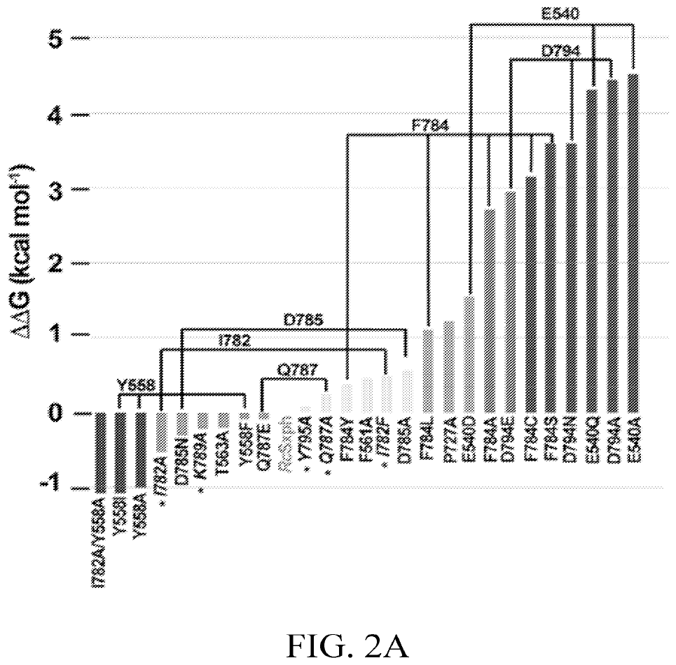

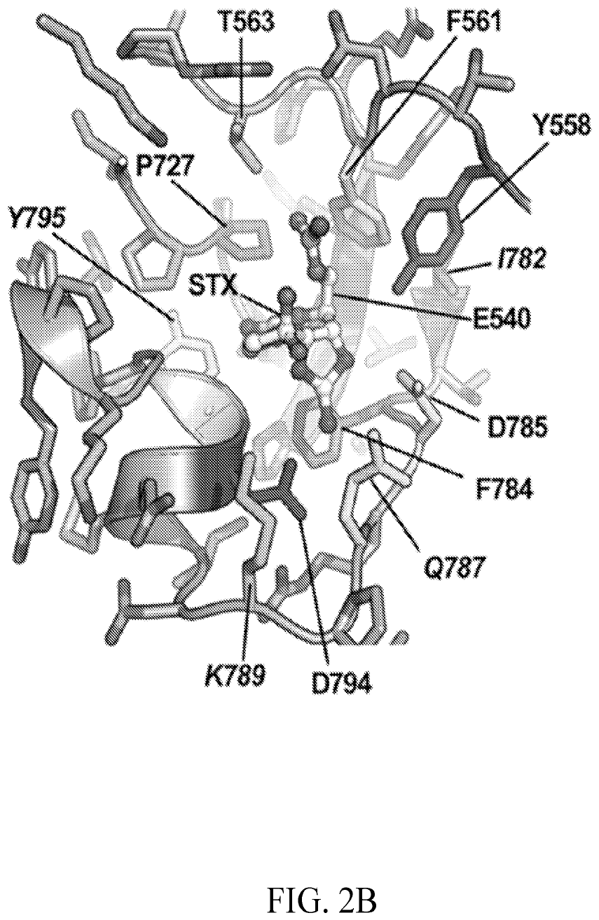

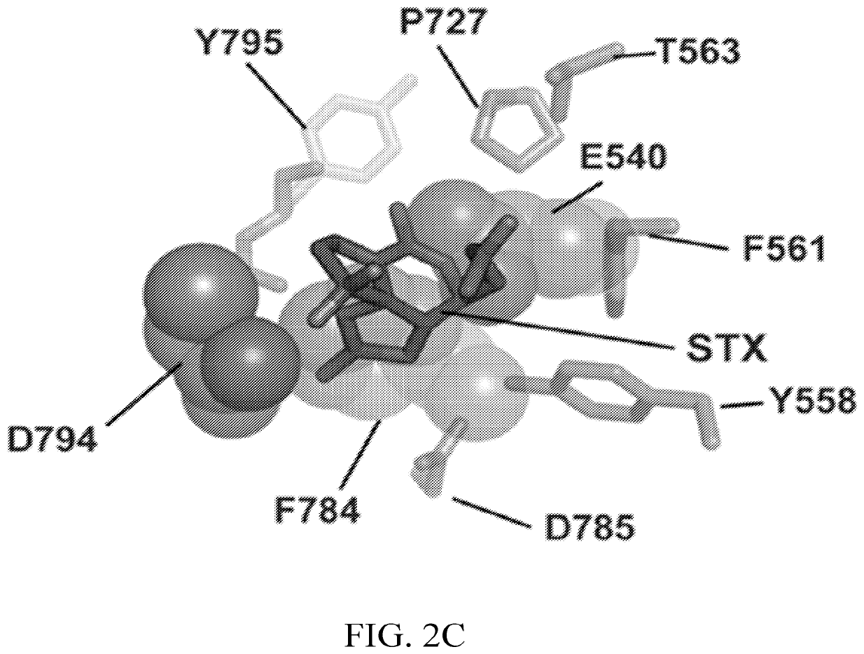

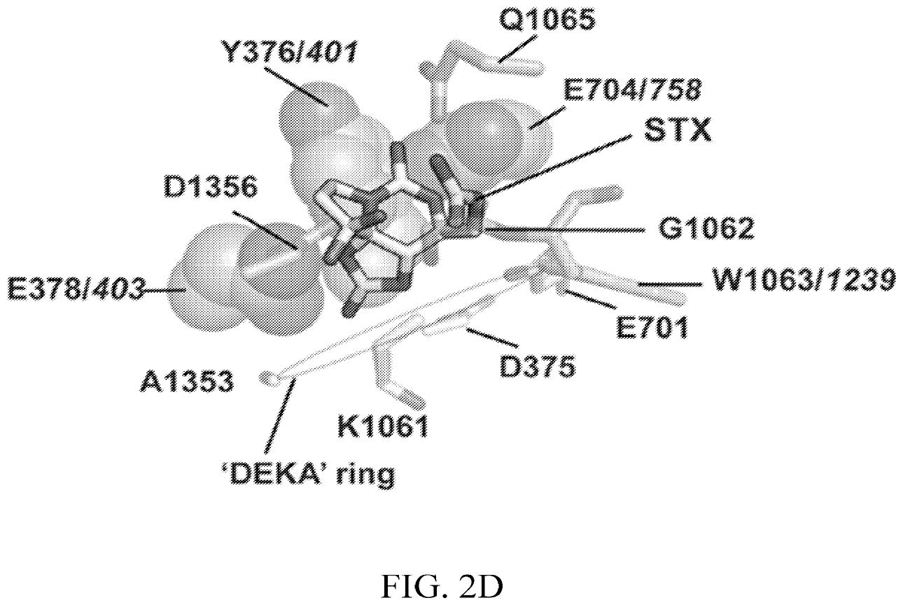

To investigate the STX affinity changes further, we used isothermal titration calorimetry (ITC) (FIG. 1F, and Table S2), a label-free method that reports directly on ligand association energetics (Velazquez-Campoy et al.), to examine the interaction of STX with RcSxph and six mutants having varied effects on binding (E540D, Y558I, Y558A, F561A, P727A, and D794E) (FIGS. 1F, 2A, S5A-C, and Table S2). Experiments with RcSxph confirm the 1:1 stoichiometry and high affinity of the RcSxph: STX interaction (Kd˜nM) reported previously (Mahar et al., Yen et al., Llewellyn and Moczydlowski) and reveal a large, favorable binding enthalpy (DH −16.1±0.2 kcal mol⁻¹) in line with previous radioligand binding studies (Llewellyn and Moczydlowski). In almost all mutants, binding affinity loss correlated with a reduction in enthalpy, consistent with a loss of interactions (Table S2). The one exception to this trend is E540D for which STX association yielded a binding enthalpy (DH −16.3±1.7 kcal mol⁻¹) very similar to wild type RcSxph that was offset by a ˜two-fold unfavorable change in binding entropy. The ITC measurements were unable to measure the affinity enhancement for Y558A and Y558I accurately due to the fact that these mutants, as well as RcSxph have Kds at the detection limit of direct titration methods (˜1 nM) (Velazquez-Campoy et al.). Nevertheless, DG_{ITC} from mutants having STX Kds within the ITC dynamic range (Kds ˜30-300 nM) showed an excellent agreement with DG_{FP} measurements made with F-STX (FIG. 1G). These data further validate the TF and FP assay trends and support the conclusion that RcSpxh:F-STX binding interactions are very similar to the RcSpxh:STX interactions. Together, these three assays (FIGS. 1E and G) provide a robust and versatile suite of options for characterizing STX:Sxph interactions.

### Sxph STX Binding Code is Focused on Two Sets of ‘Hot Spot’ Residues

To understand the structural code underlying STX binding, we classified the effects of the alanine mutations into six groups based on DDG values (FIG. 2A and Table 1) and mapped these onto the RcSxph structure (FIG. 2B). This analysis identified a binding ‘hot spot’ comprising three residues that directly contact the STX bis-guanidinium core (Glu540, Phe784, and Asp794) (Yen et al.) and an additional site near the carbamate (Pro727) where alanine mutations caused substantial STX binding losses (DDG≥1 kcal mol-1). Conversely, we also identified a site (Tyr558) where alanine caused a notable enhancement of STX binding (DDG≤−1 kcal mol⁻¹) (FIG. 2B, Table 1).

To examine the physicochemical nature of key residues critical for STX binding further, we made mutations at select positions guided by the alanine scan. Mutations at Glu540 and Asp794 (Yen et al.), residues involved in charge pair interactions with the STX guanidinium rings, that neutralized the sidechain while preserving shape and volume (FIG. 2A, Table 1) disrupted binding strongly, similar to their alanine counterparts (DDG=4.30 and 3.60 kcal mol⁻¹, for E540Q and D794N, respectively) (FIGS. 2A, S4, Table 1). Altering sidechain length while preserving the negative charge at these sites also greatly diminished STX affinity, but was notably less problematic at Glu540 (DDG=1.54 and 2.94 kcal mol⁻¹, for E540D and D794E, respectively). To probe contacts with Phe784, which makes a cation-p interaction (Infield et al.) with the STX five-membered guanidinium ring (Yen et al.), we tested changes that preserved this interface (F784Y), maintained sidechain volume and hydrophobicity (F784L), and that mimicked substitutions (F784C and F784S) found in the analogous residue in STX-resistant Na_{V}s (Na_{V}1.5, Na_{V}1.8 and Na_{V}1.9) (Yen et al., Infield et al., Heinemann et al., Satin et al., Leffler et al., Sivilotti et al.) (FIG. 2A, Table 1). Preserving the cation-p interaction with F784Y caused a modest binding reduction (DDG=0.37 kcal mol⁻¹), whereas F784L was disruptive (DDG=1.11 kcal mol⁻¹) and F784C and F784S were even more destabilizing than F784A (DDG=3.15, 3.60, and 2.71 kcal mol⁻¹, respectively).

We also examined two other positions that form part of the Sxph binding pocket near the five-membered STX guanidinium ring. Asp785 undergoes the most dramatic conformational change of any residue associated with STX binding moving from an external facing conformation to one that engages this STX element (Yen et al.). Surprisingly, D785A and D785N mutations caused only relatively modest binding changes (FIG. 2A, Table 1) (DDG=0.57 and −0.30 kcal mol⁻¹, for D785A and D785N, respectively). Because of the proximity of the second shell residue Gln787 to Asp785 and Asp794 (FIG. 2B), two residues that coordinate the five-membered STX guanidinium ring (Yen et al.), we also asked whether adding additional negative charge to this part of the STX binding pocket would enhance toxin binding affinity. However, Q787E had essentially no effect on binding (DDG=−0.09 kcal mol⁻¹).

Two residues, Tyr558 and Ile782, stood out as sites where alanine substitutions enhanced STX affinity (FIGS. 2A-B, Table 1). Tyr558 interacts with both the STX five-membered guanidinium ring and carbamate and moves away from the STX binding pocket upon toxin binding (Yen et al.), whereas Ile782 is a second shell site that buttresses Tyr558. Hence, we hypothesized that affinity enhancements observed in the Tyr558 and Ile782 mutants resulted from the reduction of Tyr558-STX clashes. In accord with this idea, Y558F had little effect on STX binding (DDG=−0.10 kcal mol⁻¹), whereas shortening the sidechain but preserving its hydrophobic character, Y558I, enhanced binding as much as Y558A (DDG=−1.07 and −1.00 kcal mol⁻¹, respectively). Increasing the sidechain volume at the buttressing position, I782F, reduced STX binding affinity (DDG=0.46 kcal mol⁻¹). Combining the two affinity enhancing mutants, Y558A/I782A, yielded only a marginal increase in affinity in comparison to Y558A (DDG=−1.07 and −1.00 kcal mol⁻¹, respectively) but was better than I782A alone (DDG=−0.53 kcal mol⁻¹). This non-additivity in binding energetics (Wells, 1990) is in line with the physical interaction of the two sites and the direct contacts of Tyr558 with the toxin. Together, these data support the idea that the Tyr558 clash with STX is key factor affecting STX affinity and suggest that it should be possible to engineer Sxph variants with enhanced binding properties by altering this site.

Collectively, this energetic map of the RcSxph STX binding pocket highlights the importance of two amino acid triads. One (Glu540, Phe784, and Asp794) engages the STX bis-guanidinium core of the toxin. The second (Tyr558, Phe561, and Pro727), forms the surface surrounding the carbamate unit (FIG. 2B). The central role of the Glu540/Phe784/Asp794 triad in the energetics of binding the bis-guanidinium STX core underscores the toxin receptor site similarities between RcSxph and Na_{V}s (Yen et al.) (FIGS. 2C-D). In both, STX binding relies on two acidic residues that coordinate the five and six-membered STX rings (Sxph Asp794 and Glu540, and rat Na_{V}1.4 Glu403 and Glu758 (Thomas-Tran and DuBois)), and a cation-p interaction (Sxph Phe784 and rat Na_{V}1.4 Tyr401 (Thomas-Tran and DuBois) and its equivalents in other Na_{V}s (Heinemann et al., Satin et al., Leffler et al., Sivilotti et al.)). Hence, both the basic architecture and binding energetics appear to be conserved even though the overall protein structures presenting these elements are dramatically different.

### Structures of Enhanced Affinity RcSxph Mutants

To investigate the structural underpinnings of the affinity enhancement caused by mutations at the Tyr558 site, we determined crystal structures of RcSxph-Y558A and RcSxph-Y558I alone (2.60 Å and 2.70 Å resolution, respectively) and as co-crystallized STX complexes (2.60 Å and 2.15 Å, respectively) (FIG. S6A-D, Table S1). Comparison of the apo- and STX-bound structures reveals little movement in the STX binding pocket upon ligand binding (RMSD_{Ca}=0.209 Å and 0.308 Å comparing apo- and STX-bound RcSxph-Y558A and RcSxph-Y558I, respectively) (FIGS. 3A-B, Supplementary movies, M1 and M2). In both, the largest conformational change is the rotation of Asp785 into the binding pocket to interact with the five-membered guanidinium ring of STX, as seen for RcSxph (FIG. 3C) (Yen et al.). By contrast, unlike in RcSxph, there is minimal movement of residue 558 and its supporting loop, indicating that both Y558A and Y558I eliminate the clash incurred by the Tyr558 sidechain. Comparison with the RcSxph:STX complex also shows that the STX carbamate in both structures has moved into a pocket formed by the mutation at Tyr558 (RMSD_{Ca}=0.279 Å and 0.327 Å comparing RcSxph:STX with RcSxph-Y558A:STX and RcSxph-Y558I:STX, respectively) (FIG. 3C). This structural change involves a repositioning of the carbamate carbon by 2 Å in the RcSxph-Y558I:STX complex relative to the RcSxph:STX complex. These findings are in line with the nearly equivalent toxin binding affinities of Y558A and Y558I, as well as with the idea that changes at the Tyr558 buttressing residue, Ile782, relieve the steric clash with STX. They also demonstrate that one strategy for increasing STX affinity is to engineer a highly organized binding pocket that requires minimal conformational changes to bind STX.

TABLE S1
Crystallographic data collection and refinement statistics

| | | | RcSxph -Y588A:STX | | | | RcSxph -Y558I:STX | |
|---|---|---|---|---|---|---|---|---|
| | RcSxph -Y558A | | (co-crystal) | | RcSxph -Y558I | | (co-crystal) | |
| Data Collection | | | | | | | | |
| Space group | P2₁2₁2₁ | | P2₁2₁2₁ | | P2₁2₁2₁ | | P2₁2₁2₁ | |
| Cell dimensions a/b/c (Å) | 96.61, 109.05, 254.89 | | 95.98, 107.14, 253.04 | | 96.39, 107.15, 254.79 | | 96.03, 107.81, 253.58 | |
| ⊏/⊏/⊐⊏(°) | 90, 90, 90 | | 90, 90, 90 | | 90, 90, 90 | | 90, 90, 90 | |
| Resolution (Å) | 47.81-2.60 | (2.65-2.60) | 47.37-2.60 | (2.65-2.60) | 47.61-2.70 | (2.76-2.70) | 47.5-2.15 | (2.19-2.15) |
| Rmerge (%) | 0.108 | (4.094) | 0.115 | (3.964) | 0.159 | (4.833) | 0.089 | (1.558) |
| I/σI | 12.9 | (0.9) | 12.5 | (0.8) | 8.8 | (0.6) | 16.2 | (1.2) |
| CC(½) | 0.998 | (0.532) | 0.998 | (0.458) | 0.998 | (0.419) | 0.999 | (0.599) |
| Completeness (%) | 99.6 | (100) | 99.9 | (100) | 99.9 | (99.8) | 99.5 | (93.6) |
| Redundancy | 13.4 | (13.9) | 13.4 | (14.0) | 13.3 | (14.0) | 12.2 | (6.2) |
| Total reflections | 1116931 | (62978) | 1085831 | (61318) | 975607 | (62994) | 1736282 | (40652) |
| Unique reflections | 83173 | (4517) | 81054 | (4377) | 73319 | (4504) | 142848 | (6596) |

| Wilson B-factor | 83.42 | 84.83 | 90.34 | 44.01 |
|---|---|---|---|---|
| Wavelength (Å) | 1.033 | 1.033 | 1.033 | 1.033 |
| Refinement | | | | |
| R_{work}/R_{free} (%) | 22.79/26.37 | 23.20/26.17 | 23.42/27.21 | 20.73/23.37 |
| No. of chains in AU | 2 | 2 | 2 | 2 |
| No. of protein atoms | 12616 | 12616 | 12622 | 12622 |
| No. of ligand atoms | 0 | 42 | 0 | 72 |
| No. of water atoms | 77 | 60 | 79 | 794 |
| RMSD bond lengths (Å) | 0.002 | 0.002 | 0.003 | 0.003 |
| RMSD angles (°) | 0.50 | 0.49 | 0.55 | 0.59 |
| Ramachandran favored/ | 94.90/4.91/0.18 | 94.29/5.47/0.25 | 93.61/6.08/0.31 | 95.33/4.30/0.37 |
| allowed/outliers (%) | | | | |

TABLE S1
Crystallographic data collection and refinement statistics (continued)

| | RcSxph: F-STX | | | | NpSxph + STX | | NpSxph:F-STX | |
|---|---|---|---|---|---|---|---|---|
| | (soaked) | | NpSxph | | (co-crystal) | | (soaked) | |
| Data Collection | | | | | | | | |
| Space group | P2₁2₁2₁ | | R3 | | R3 | | R3 | |
| Cell dimensions a/b/c (Å) | 96.44, 109.37, 256.36 | | 229.046, 229.046, 67.428 | | 228.848, 228.848, 67.224 | | 229.186, 229.186, 67.347 | |
| \|/\|/\|\| (°) | 90, 90, 90 | | 90, 90, 120 | | 90, 90, 120 | | 90, 90, 120 | |
| Resolution (Å) | 47.97-2.65 | (2.70-2.65) | 43.29-2.2 | (2.279-2.2) | 39.88-2.0 | (2.071-2.0) | 42.62-2.2 | (2.279-2.2) |
| Rmerge (%) | 0.112 | (4.136) | 0.05218 | (0.8687) | 0.05456 | (0.9961) | 0.07078 | (1.889) |
| I/σI | 11.1 | (0.8) | 12.66 | (0.89) | 14.11 | (1.11) | 19.64 | (1.35) |
| CC(½) | 0.999 | (0.513) | 0.999 | (0.465) | 0.998 | (0.637) | 0.999 | (0.602) |
| Completeness (%) | 99.9 | (99.8) | 98.60 | (91.37) | 99.89 | (99.61) | 99.90 | (99.97) |
| Redundancy | 10.4 | (11.0) | 3.3 | (2.1) | 5.2 | (4.8) | 10.6 | (11.0) |
| Total reflections | 830262 | (49019) | 219806 | (12669) | 459480 | (42277) | 707304 | (73200) |
| Unique reflections | 79524 | (4469) | 66067 | (6127) | 88641 | (8856) | 66898 | (6682) |

| Wilson B-factor | 83.73 | 52.73 | 49.89 | 55.11 |
|---|---|---|---|---|
| Wavelength (Å) | 1.033 | 1.033167 | 1.033167 | 1.033167 |
| Refinement | | | | |
| R_{work}/R_{free} (%) | 23.46/26.51 | 19.50/23.55 | 19.25/22.09 | 19.64/24.26 |
| No. of chains in AU | 2 | 1 | 1 | 1 |
| No. of protein atoms | 12630 | 6373 | 6385 | 6346 |
| No. of ligand atoms | 110 | 38 | 59 | 93 |
| No. of water atoms | 88 | 229 | 287 | 161 |
| RMSD bond lengths (Å) | 0.002 | 0.004 | 0.004 | 0.006 |
| RMSD angles (°) | 0.50 | 0.60 | 0.62 | 0.75 |
| Ramachandran favored/ | 94.23/5.41/0.37 | 95.71/4.17/0.12 | 95.85/3.90/0.24 | 94.19/5.56/0.25 |
| allowed/outliers (%) | | | | |

Sxph STX Binding Affinity Changes Alter Na, Rescue from STX Block

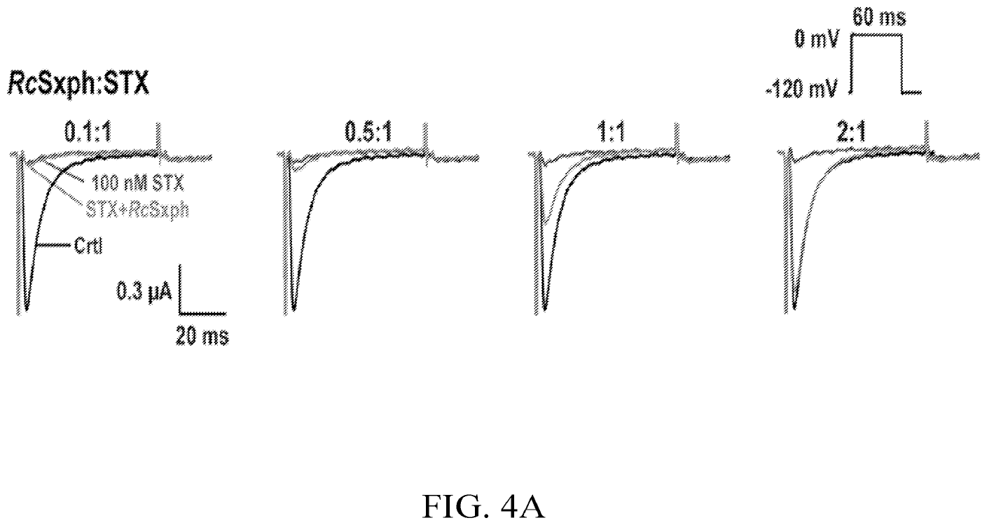

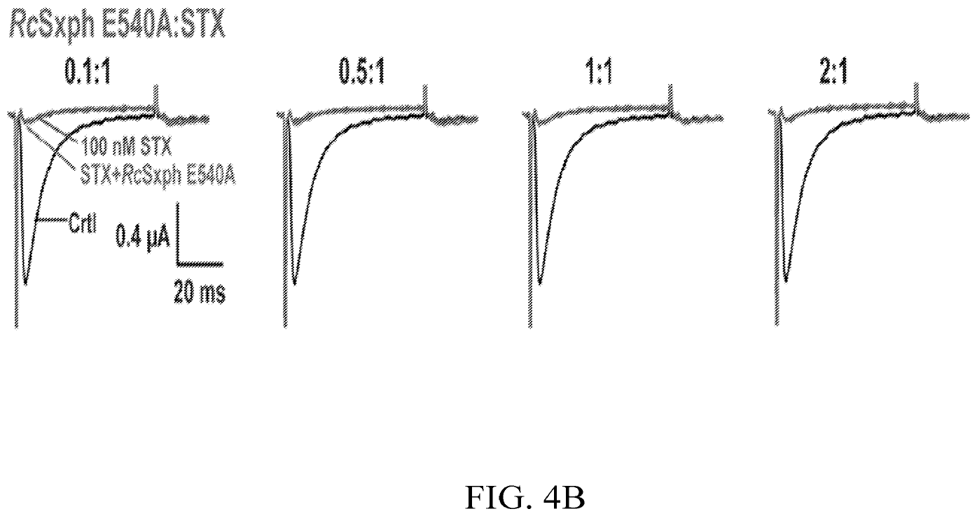

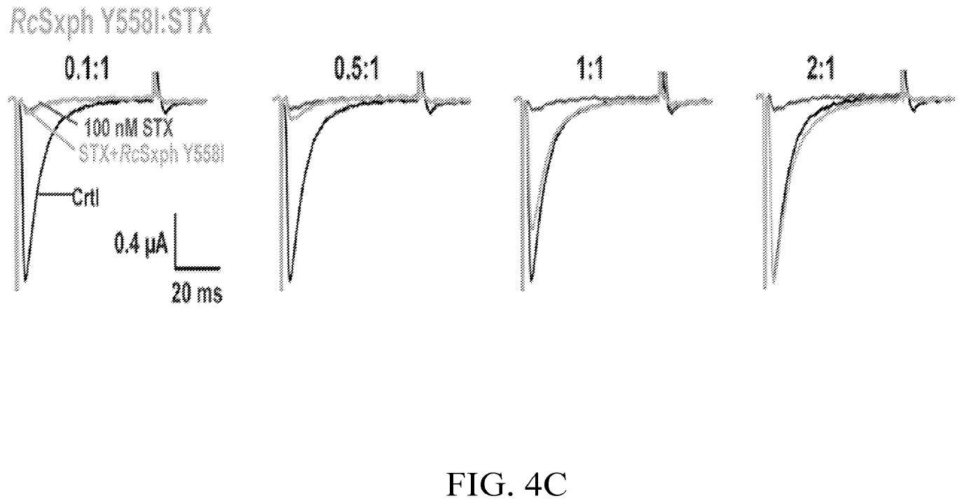

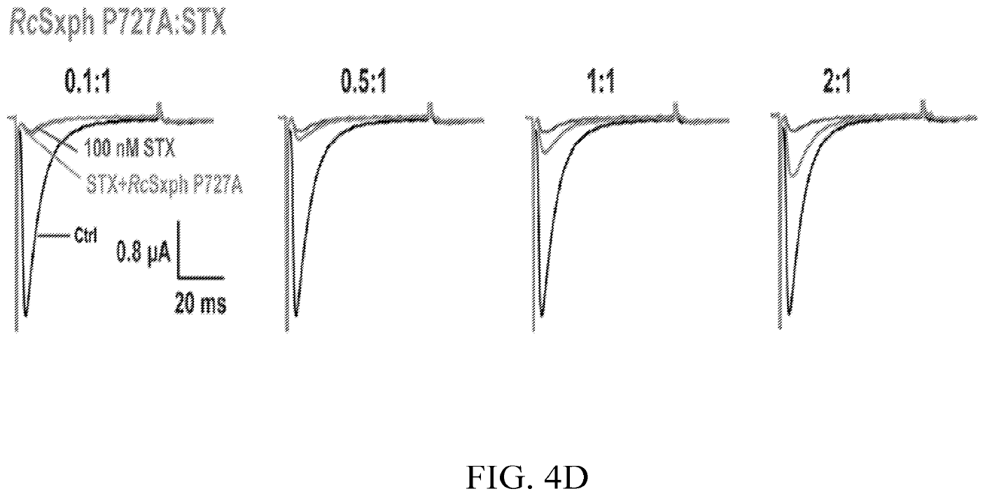

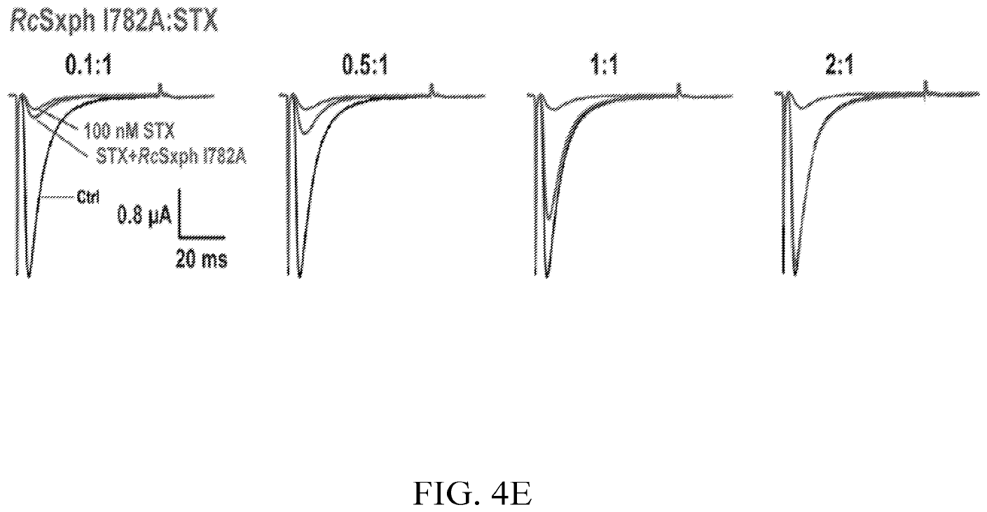

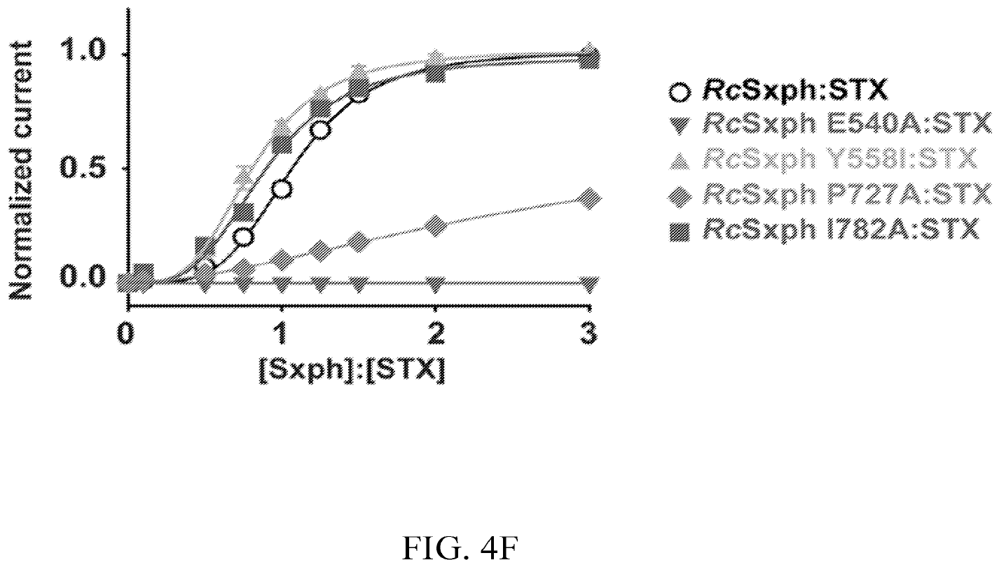

RcSxph acts as a ‘toxin sponge’ that can reverse STX inhibition of Na_{V}s (Abderemane-Ali et al.). To test the extent to which this property is linked to the intrinsic affinity of RcSxph for STX, we evaluated how STX affinity altering mutations affected RcSxph rescue of channels blocked by STX. As shown previously, titration of different RcSxph:STX ratios against Phyllobates terribilis Na_{V}1.4 (PtNa_{V}1.4), a Na_{V} having an IC₅₀ for STX of 12.6 nM (Abderemane-Ali et al.), completely reverses the effects of STX at ratios of 2:1 RcSxph:STX or greater (FIGS. 4A and F). Incorporation of mutations that affect STX affinity altered the ability of RcSxph to rescue Na_{V}s and followed the binding assay trends. Mutants that increased STX affinity, Y558I and I782A, improved the ability of RcSxph to rescue PtNa_{V}1.4 (Effective Rescue Ratio₅₀ (ERR₅₀)=0.81±0.01, 0.87±0.02, and 1.07±0.02 for Y558I, I782A, and RcSxph, respectively), whereas mutations that compromised STX binding reduced (P727A, ERR₅₀>4) or eliminated (E540A) the ability of RcSxph to reverse the STX inhibition (FIG. 4B-F). This strong correlation indicates that the ‘toxin sponge’ property of Sxph (Abderemane-Ali et al.) depends on the capacity of Sxph to sequester STX and adds further support to the idea that Sxph has a role in toxin resistance mechanisms (Mahar et al., Abderemane-Ali et al.).

### Expansion of the Sxph Family

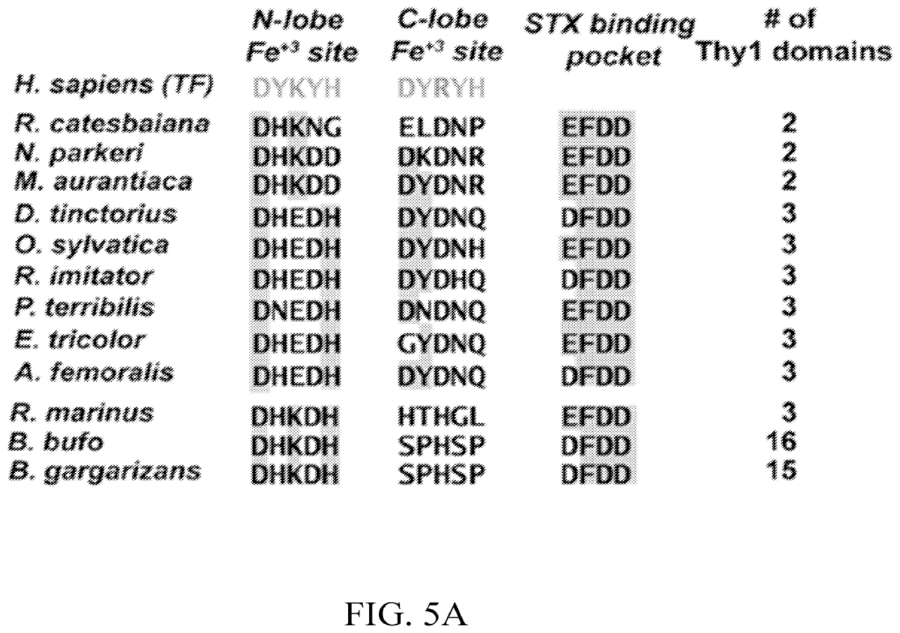

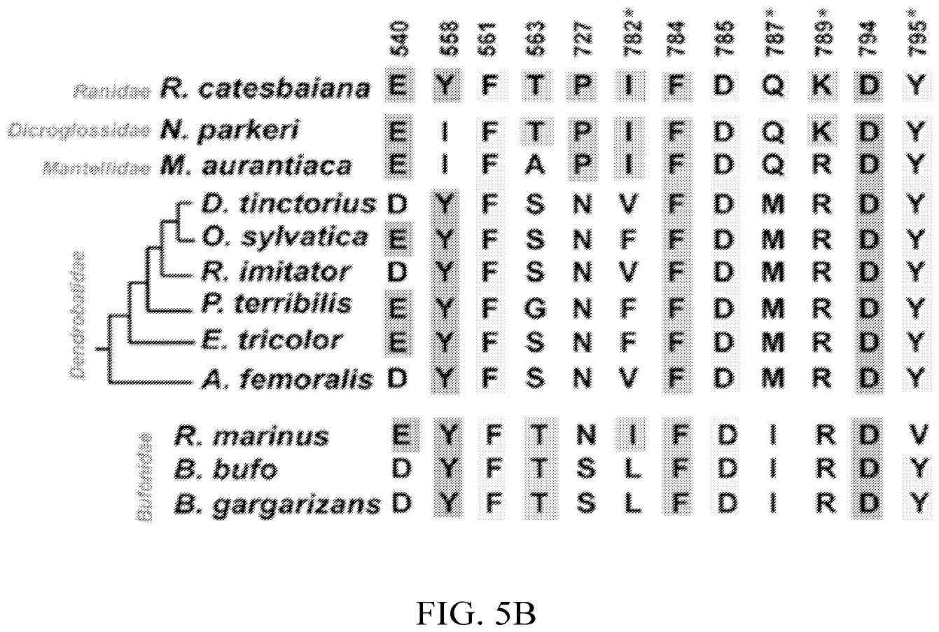

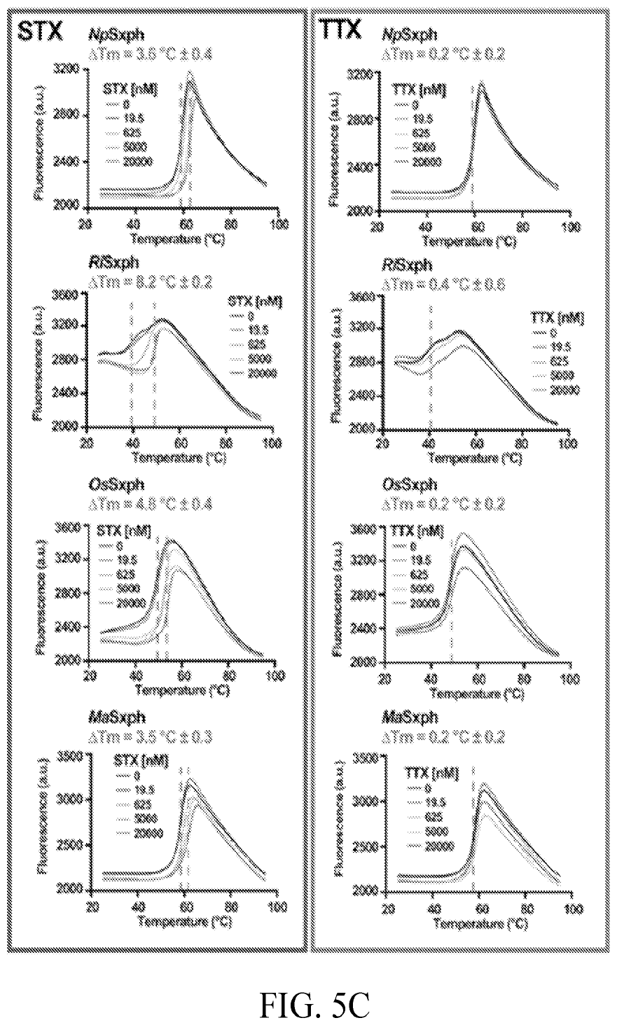

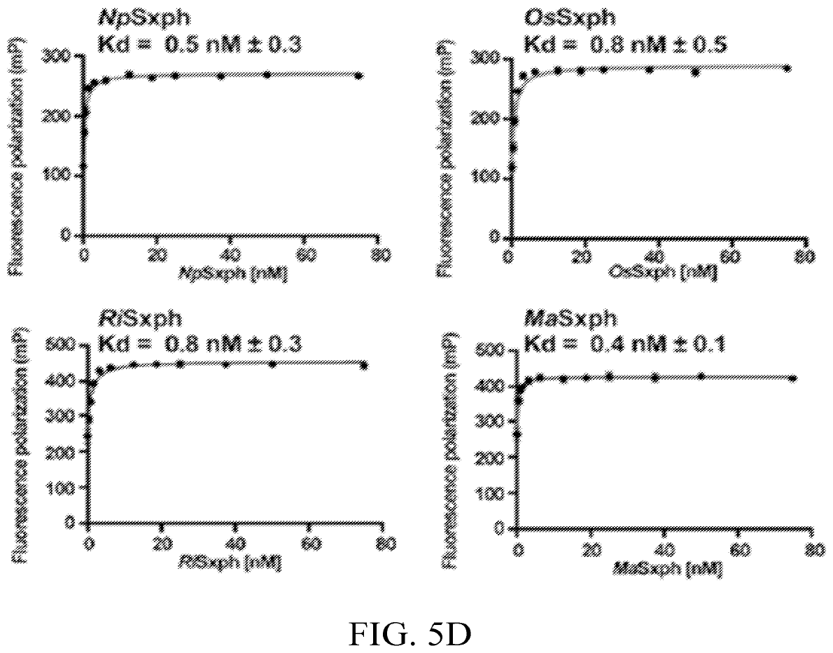

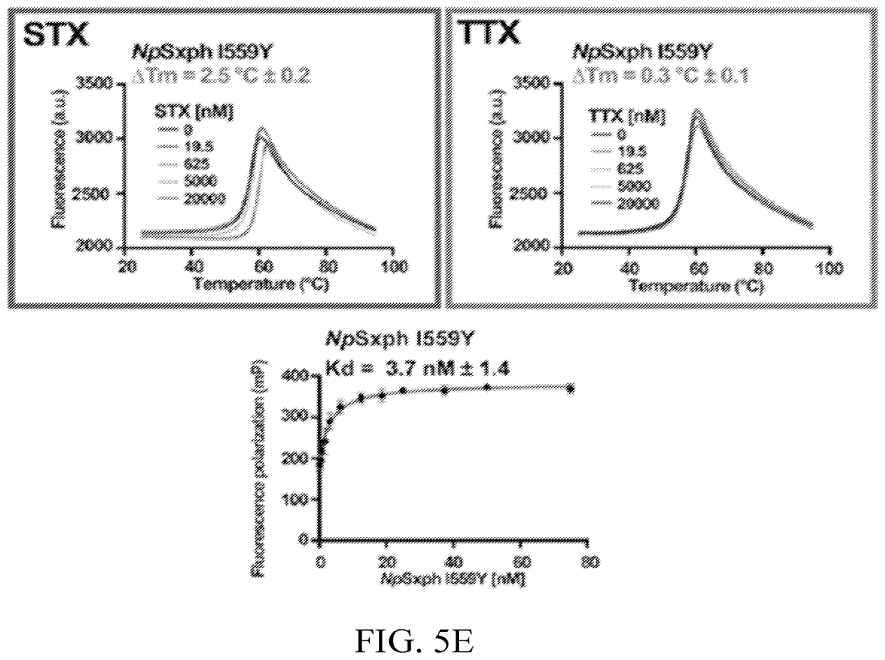

STX binding activity has been reported in the plasma, hemolymph, and tissues of diverse arthropods, amphibians, fish, and reptiles (Doyle et al., Llewellyn et al.), suggesting that many organisms harbor Sxph-like proteins. Besides RcSpxh, similar Sxphs have been identified in only two other frogs, the High Himalaya frog Nanorana parkeri (Yen et al.) and the little devil poison frog Oophaga sylvatica (Caty et al.). As a number of poison frogs exhibit resistance to STX poisoning (Abderemane-Ali et al.), we asked whether the STX binding site ‘recognition code’ could enable identification of Sxph homologs in other amphibians. To this end, we determined the sequences of ten new Sxphs (FIGS. 5A and B, FIG. S7, and FIG. S8). These include six Sxphs in two poison dart frog families (Family Dendrobatidae: Dyeing poison dart frog, Dendrobates tinctorius; Little devil poison frog, O. sylvatica; Mimic poison frog, Ranitomeya imitator; Golden dart frog, Phyllobates terribilis; Phantasmal poison frog, Epipedobates tricolor; and Brilliant-thighed poison frog, Allobates femoralis; and Family: Mantellidae Golden mantella, Mantella aurantiaca), and three Sxphs in toads (Caucasian toad, Bufo; Asiatic toad, Bufo gargarizans; and South American cane toad, Rhinella marina). The identification of the OsSxph sequence confirms its prior identification by mass spectrometry (Caty et al.) and the discovery of RmSxph agrees with prior reports of Sxph-like STX binding activity in the cane toad (R. marinus) (Llewellyn et al., Tanaka et al.).

Sequence comparisons (FIGS. S7 and S8) show that all of the new Sxphs share the transferrin fold found in RcSxph comprising N- and C-lobes each having two subdomains (N1, N2 and C1, C2, respectively) (Yen et al., Morabito and Moczdlowski) and the signature ‘EFDD’ motif (Yen et al.) or a close variant in the core of the C-lobe STX binding site (FIG. 5A). Similar to RcSxph, the new Sxphs also have amino acid differences relative to transferrin that should eliminate Fe³⁺ binding (Yen et al., Morabito and Moczdlowski, Li et al.), as well as a number of protease inhibitor thyroglobulin domains (Thy1) inserted between the N1 and N2 N-lobe subdomains (Yen et al., Lenarčič et al.) (FIG. 5A and FIGS. S7-S9). These Thy1 domain insertions range from two in RcSxph, NpSxph, and MaSxph, to three in the dendrobatid poison frog and cane toad Sxphs, to 16 and 15 in toad BbSxph and BgSxph, respectively (FIG. 5A and FIGS. S7-S9).

We used the STX recognition code defined by our studies as a template for investigating cross-species variation in the residues that contribute to STX binding (FIG. 5B). This analysis shows a conservation of residues that interact with the STX bis-guanidium core (Glu540, Phe784, Asp785, Asp794, and Tyr795) and carbamate (Phe561). Surprisingly, five of the Sxphs (D. tinctorius, R. imitator, A. femoralis, B. bufo, and B. gargarizans) have an aspartate instead of a glutamate at the Glu540 position in RcSxph that contributes the most binding energy (FIG. 2A). The equivalent change in RcSxph, E540D, reduced STX affinity by ˜100 fold (Table 1) and uniquely alters enthalpy and entropy binding parameters compared to other affinity-lowering mutations (Table S2). Additionally, we identified variations at two sites for which mutations increase RcSxph STX binding, Tyr558 and Ile782 (FIGS. 2A, 4C, 4E, and Table 1). NpSxph and MaSxph have an Ile at the Tyr558 site, whereas eight of the new Sxphs have hydrophobic substitutions at the Ile782 position (FIG. 5B). The striking conservation of the Sxph scaffold and STX binding site indicate that this class of ‘toxin sponge’ proteins is widespread among diverse Anurans, while the amino acid variations in key positions (Glu540, Tyr558, and Ile782), raise the possibility that the different Sxph homologs have varied STX affinity or selectivity for STX congeners.

TABLE S2
RcSxph:STX and NpSxph:STX thermodynamic binding parameters

| | | | | ΔH (kcal | ΔS (cal | ΔG (kcal | |
|---|---|---|---|---|---|---|---|
| | | N (sites) | Kd (nM) | mol⁻¹) | mol⁻¹ K⁻¹) | mol⁻¹) | n |
| RcSxph | WT | 1.02 ± 0.01 | 1.2 ± 0.8 | −16.1 ± 0.2 | −12.7 ± 0.9 | −12.3 ± 0.5 | 3 |
| | Y558A | 1.01 ± 0.01 | 1.2 ± 0.4 | −15.3 ± 0.0 | −11.1 ± 1.1 | −12.2 ± 0.2 | 2 |
| | Y558I | 1.05 ± 0.03 | 1.1 ± 0.5 | −15.5 ± 0.3 | −10.5 ± 0.6 | −12.2 ± 0.3 | 2 |
| | F561A | 1.07 ± 0.01 | 13.4 ± 1.4 | −12.7 ± 0.2 | −6.6 ± 0.9 | −10.8 ± 0.1 | 3 |
| | P727A | 0.97 ± 0.03 | 31.3 ± 11.6 | −11.5 ± 0.0 | −4.2 ± 0.9 | −10.3 ± 0.2 | 2 |
| | E540D | 0.98 ± 0.02 | 68.9 ± 10.7 | −16.3 ± 1.7 | −21.9 ± 5.7 | −9.8 ± 0.1 | 4 |
| | D794E | 0.99 ± 0.01 | 312.5 ± 2.9 | −11.8 ± 0.0 | −8.9 ± 0.1 | −8.9 ± 0.1 | 2 |
| NpSxph | WT | 0.92 ± 0.02 | 2.5 ± 0.1 | −18.7 ± 0.2 | −23.2 ± 0.8 | −11.8 ± 0.1 | 2 |
| | I559Y | 0.94 ± 0.03 | 2.5 ± 0.8 | −16.8 ± 0.2 | −16.9 ± 1.1 | −11.8 ± 0.2 | 4 |

Diverse Sxph Family Members have Conserved STX Binding Properties

To explore the STX binding properties of this new set of Sxphs and to begin to understand whether changes in the binding site composition affect toxin affinity, we expressed and purified four representative variants. These included two Sxphs having STX binding site sequences similar to RcSxph (NpSxph and MaSxph) and two Sxphs bearing more diverse amino acid differences (RiSxph and OsSxph), including one displaying the E540D substitution (RiSxph). This set also represents Sxphs having either two Thy1 domains similar to RcSxph (NpSxph and MaSxph) or three Thy1 domains (OsSxph and RiSxph) (FIG. 5A). TF experiments showed STX-dependent DTms for all four Sxphs. By contrast, equivalent concentrations of TTX had no effect (FIG. 5C), indicating that, similar to RcSpxh (FIG. 1A) (Mahar et al., Abderemane-Ali et al.), all four Sxphs bind STX but not TTX. Unlike the other Sxphs, the RiSxph melting curve showed two thermal transitions; however, only the first transition was sensitive to STX concentration (FIG. 5C). FP binding assays showed that all four Sxphs bound F-STX and revealed affinities stronger than RcSxph (FIG. 5D and Table 2). The enhanced affinity of NpSxph and MaSxph for STX relative to RcSxph is consistent with the presence of the Y558I variant (FIG. 5B). Importantly, the observation that RiSxph has a higher affinity for STX than RcSxph despite the presence of the E540D difference suggests that the other sequence variations in the RiSxph STX binding pocket compensate for this Glu→Asp change at Glu540.

TABLE 2
Comparison of Sxph STX binding properties

| Construct | Kd (nM) | ΔΔG (kcal mol⁻¹) | n |
|---|---|---|---|
| MaSxph | 0.4 ± 0.1 | −1.73 | 4 |
| NpSxph | 0.5 ± 0.3 | −1.60 | 13 |
| RiSxph | 0.8 ± 0.3 | −1.32 | 4 |
| OsSxph | 0.8 ± 0.5 | −1.32 | 10 |
| NpSxph I559Y | 3.7 ± 1.4 | −0.04 | 8 |
| RcSxph | 7.4 ± 2.6 | 0 | 10 |

Kd, dissociation constant; n, number of observations
ΔΔG = RT In (K_{d(XSxph)}/K_{d(RcSxph)}); T = 298K.
Errors for measurements are S.D.

Because NpSxph has a higher affinity for STX than RcSxph (FIGS. 1D, 5D and Table 2) and has an isoleucine at the Tyr558 site (FIG. 5B), we asked whether the NpSxph I559Y mutant that converts the NpSxph binding site to match RcSxph would lower STX affinity. TF measurements showed that NpSxph I559Y had a ˜1° C. smaller DTm than NpSxph (DTm=3.6° C.±0.4 versus 2.5° C.±0.2 for NpSxph and NpSxph I559Y, respectively), indicative of a decreased binding affinity (FIGS. 5C and E). This result was validated by FP (DDG=−1.56 kcal mol⁻¹), yielding a result of similar magnitude to the RcSxph Y558I differences (FIG. 5E, Tables 1 and 2). ITC confirmed the high affinity of the interaction (FIG. S5D-F), but could not yield an explicit Kd given its low nanomolar value (FIG. S5F). Nevertheless, these experiments validate the 1:1 stoichiometry of the STX:NpSTX interaction (Table S2) and show that the I559Y change reduced the binding enthalpy, consistent with perturbation of NpSxph:STX interactions (DH=−18.7±0.2 vs. −16.8±0.2 kcal mol⁻¹, NpSxph and NpSxph 1559Y, respectively) (Table S2). Taken together, these experiments establish the conserved nature of the STX binding pocket among diverse Sxph homologs and show that the STX recognition code derived from RcSxph studies (FIG. 5B) can identify key changes that influence toxin binding.

### Structures of Apo- and STX Bound NpSxph Reveal a Pre-Organized STX Binding Site

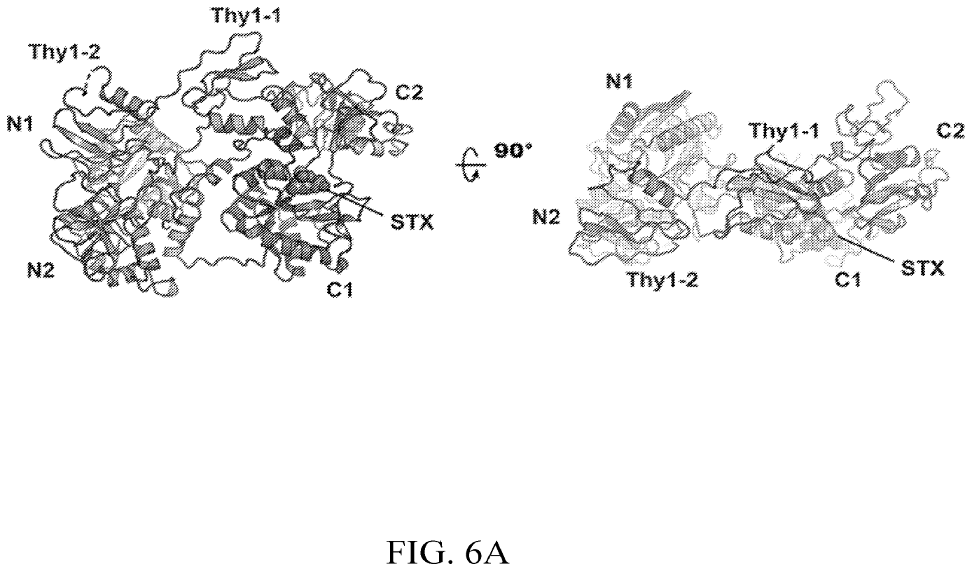

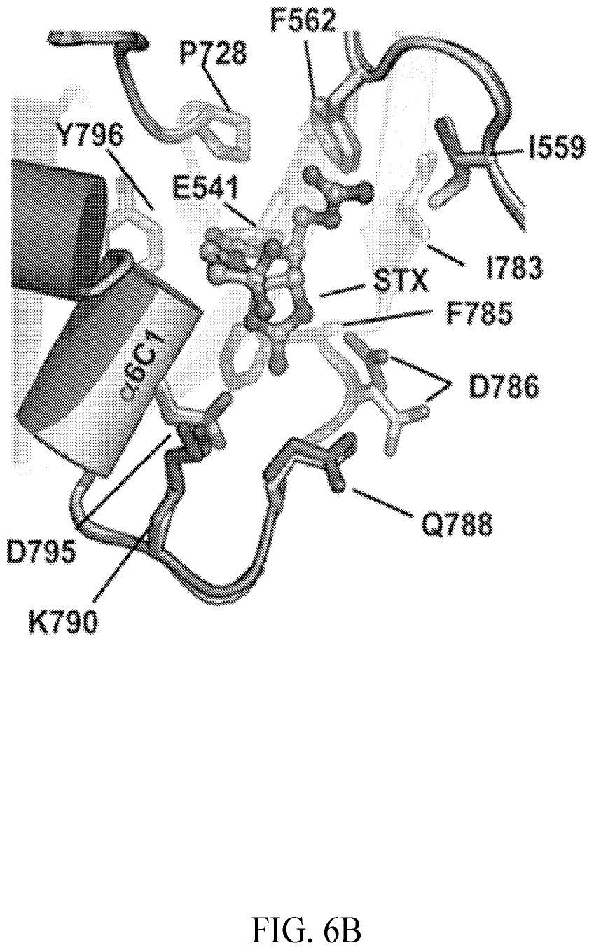

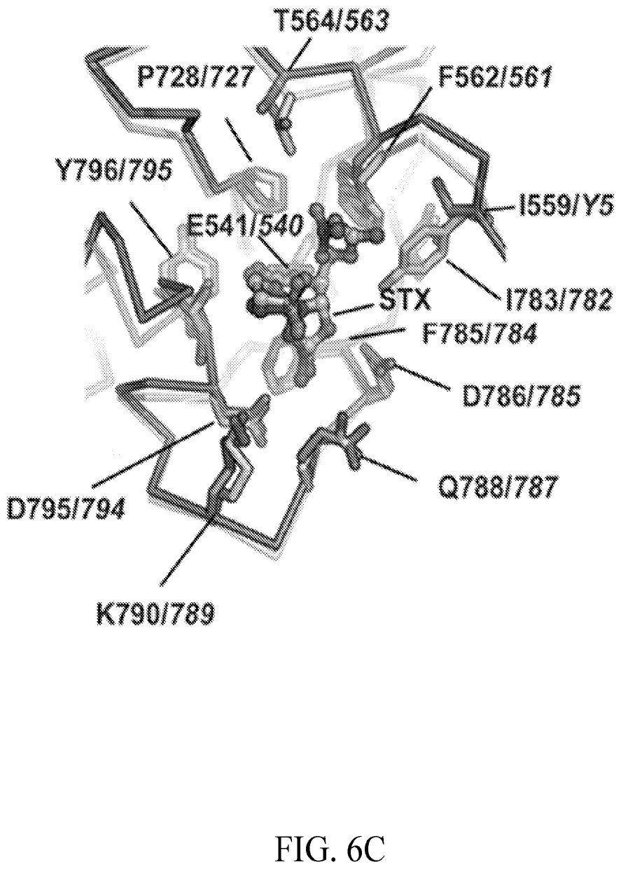

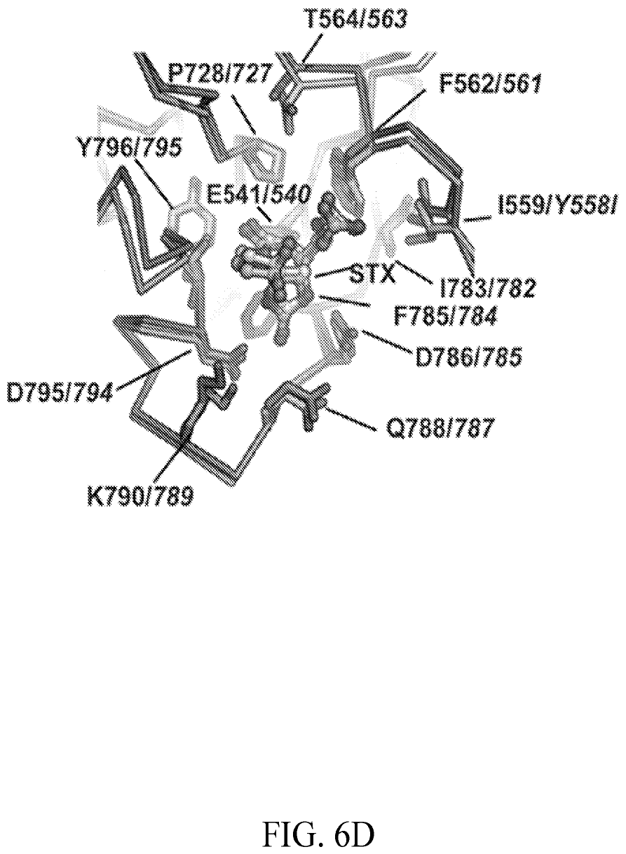

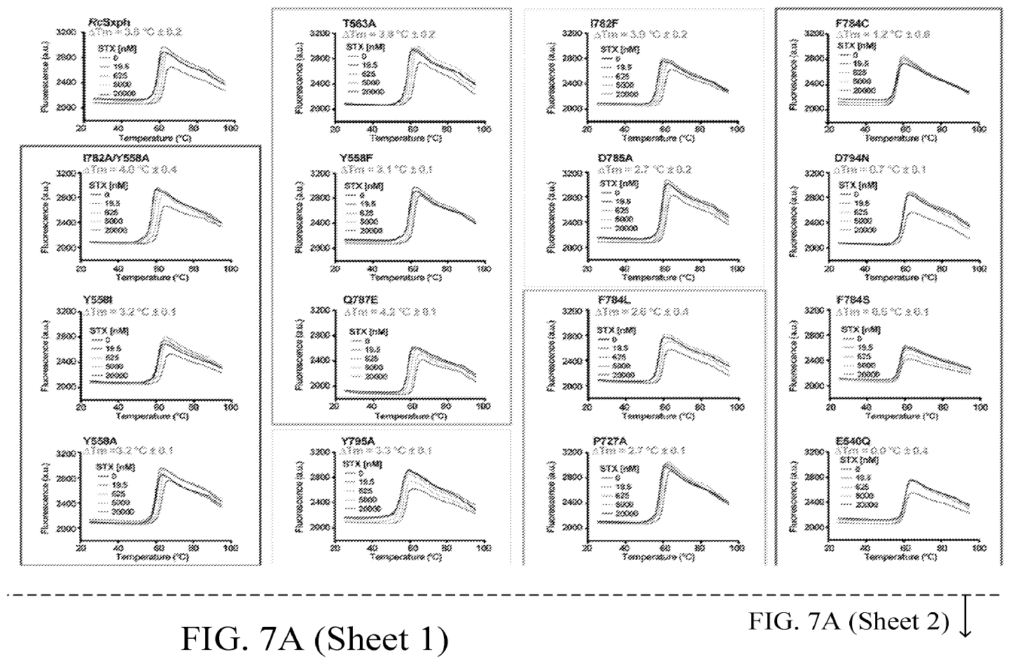

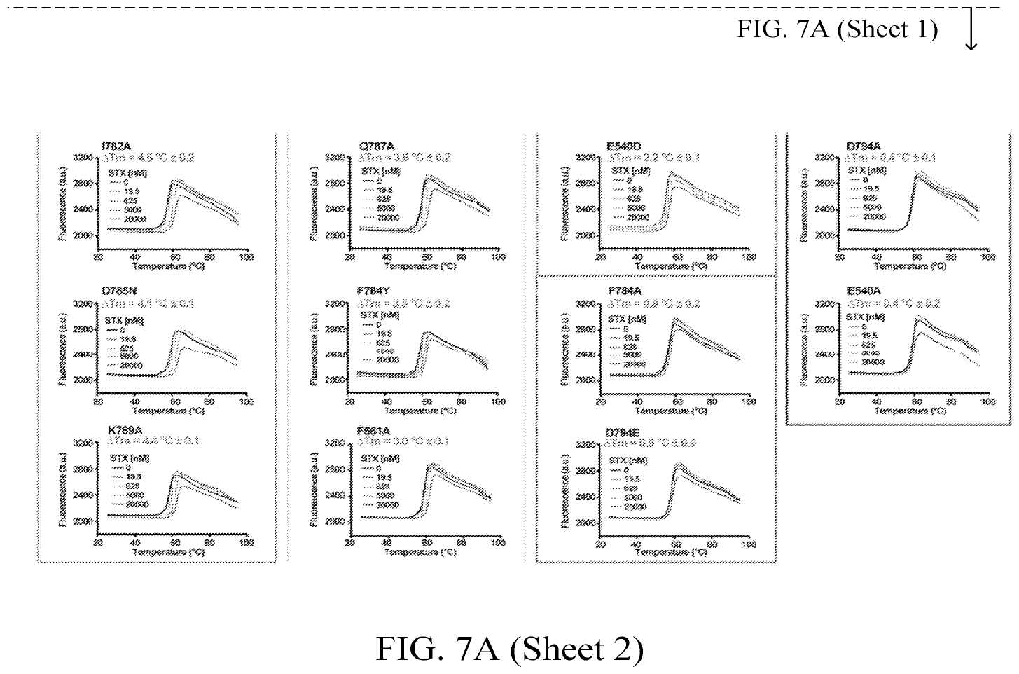

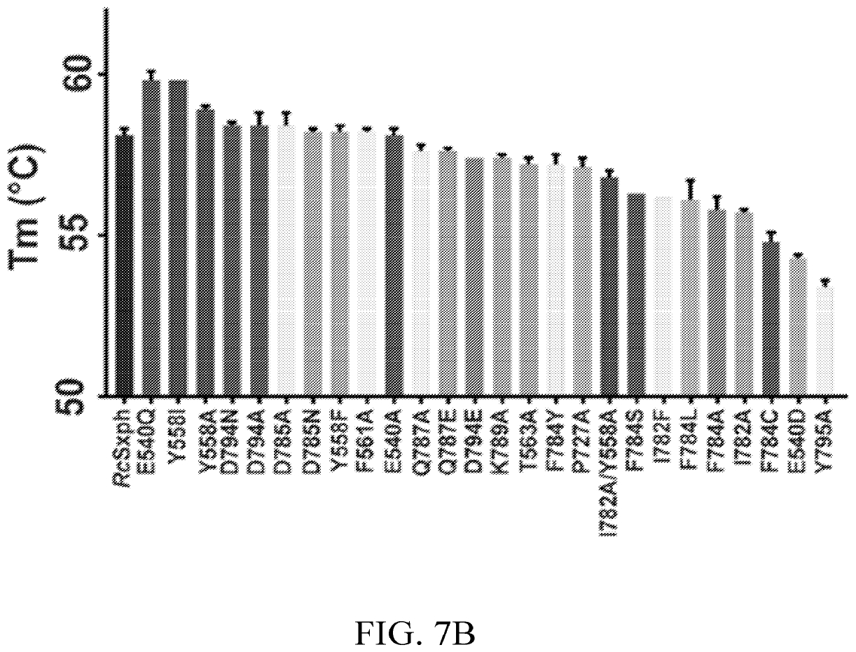

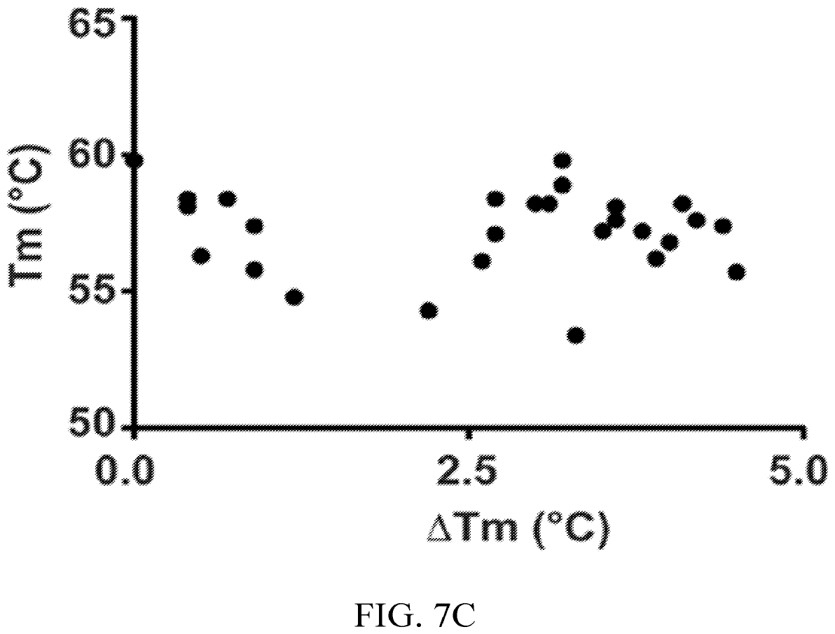

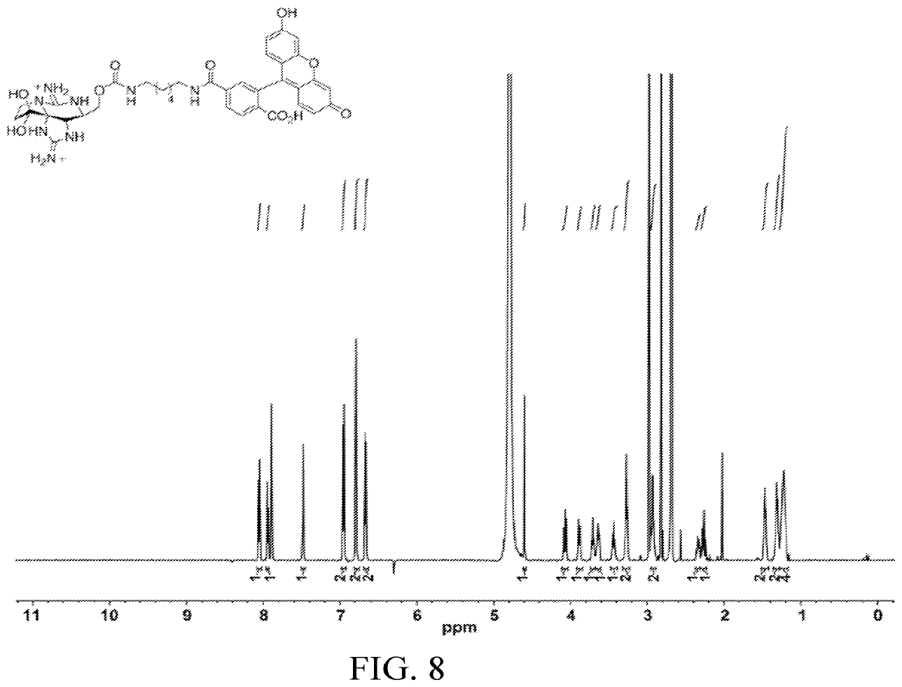

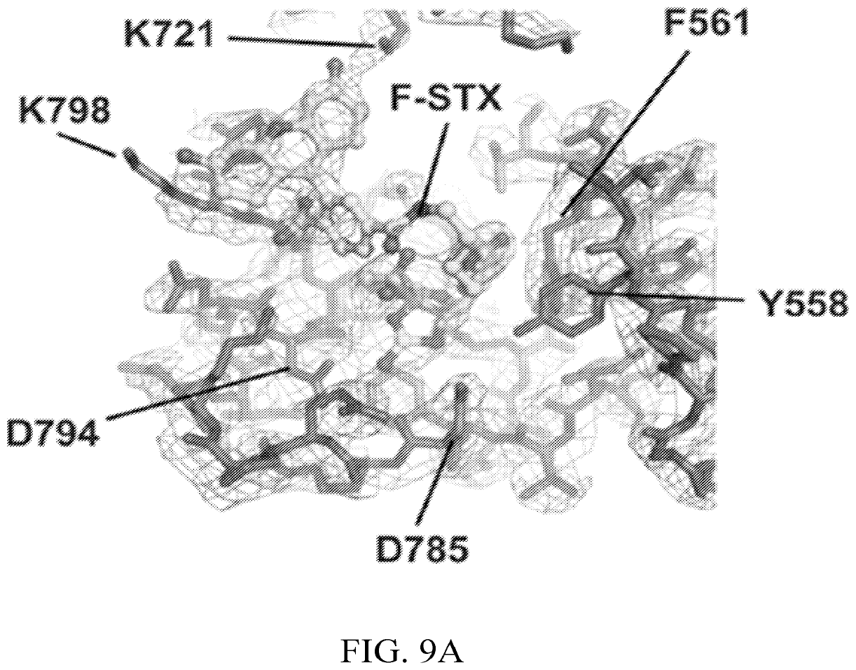

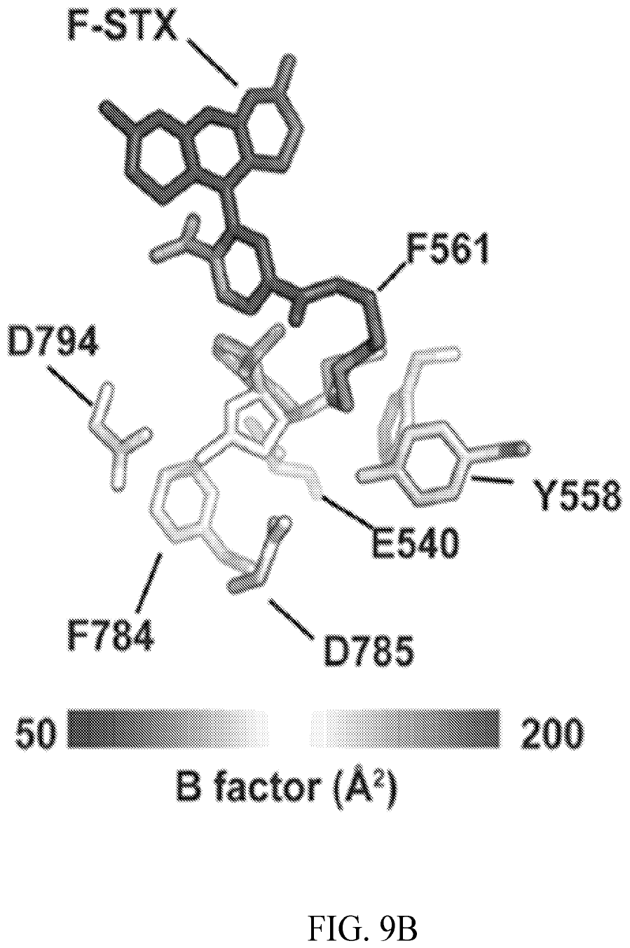

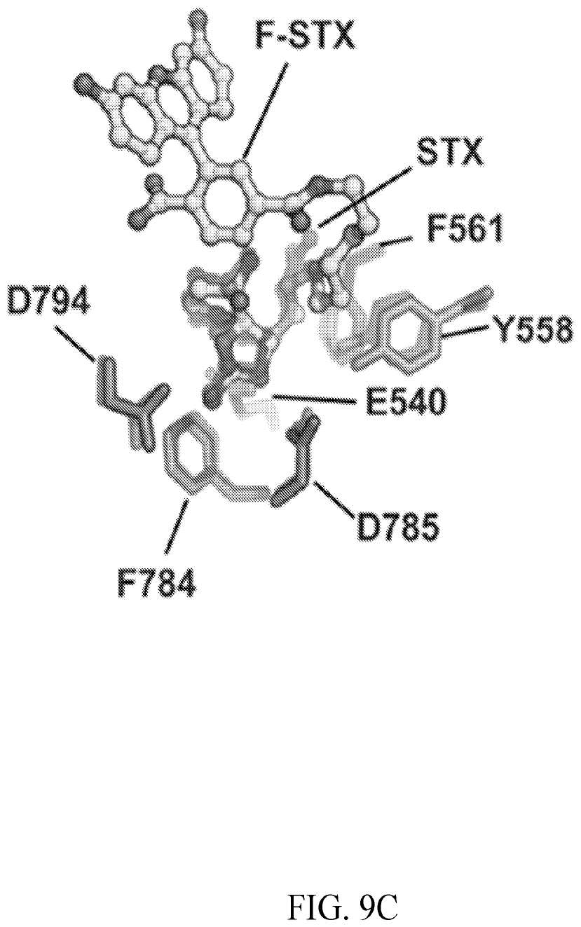

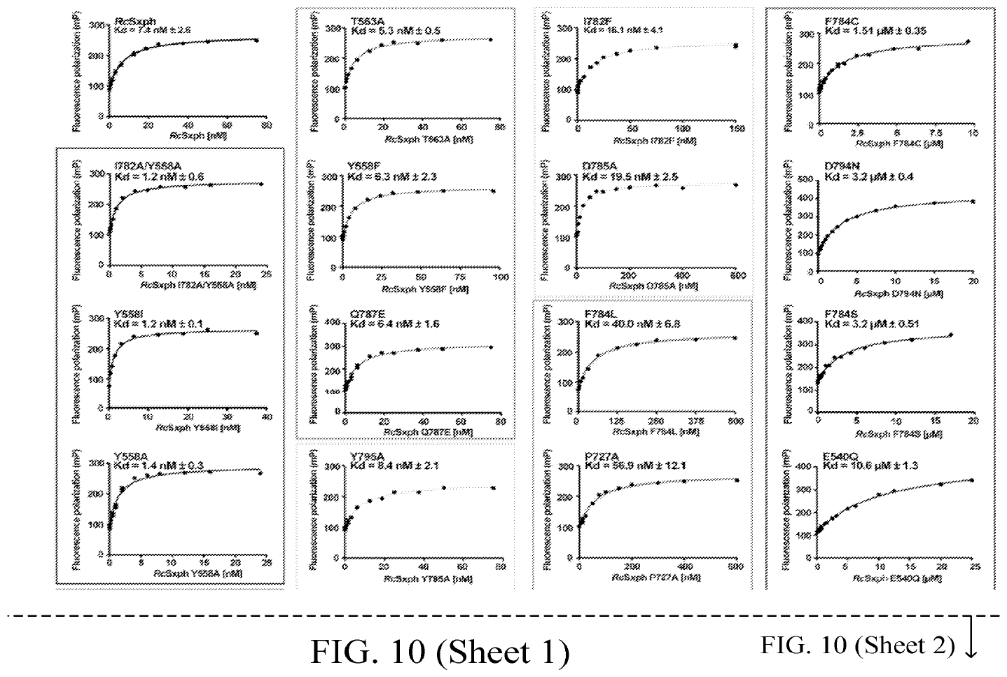

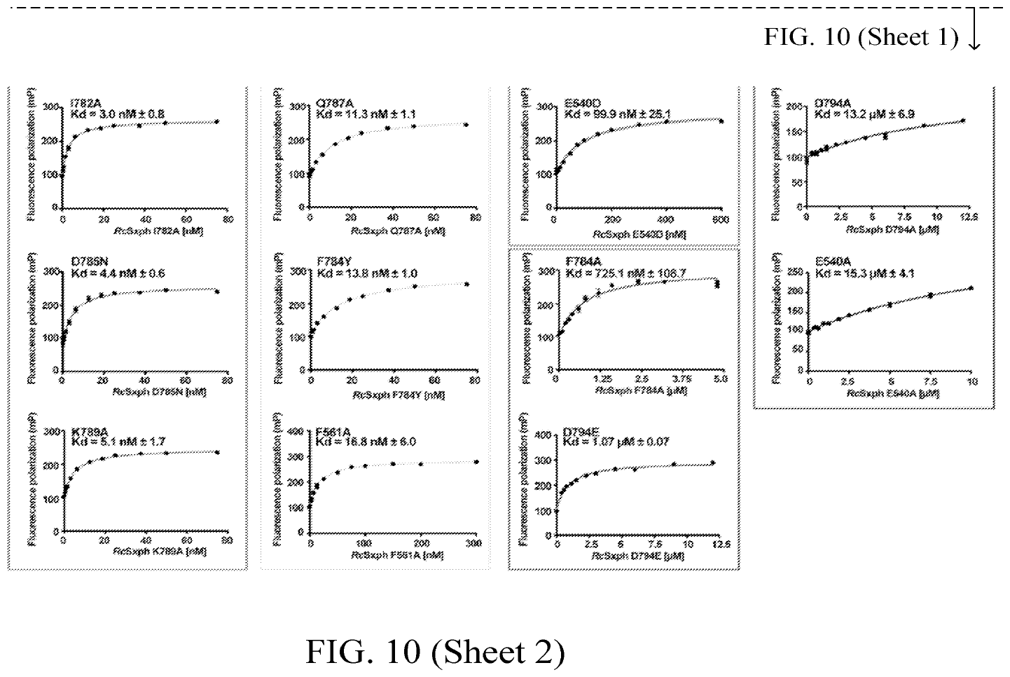

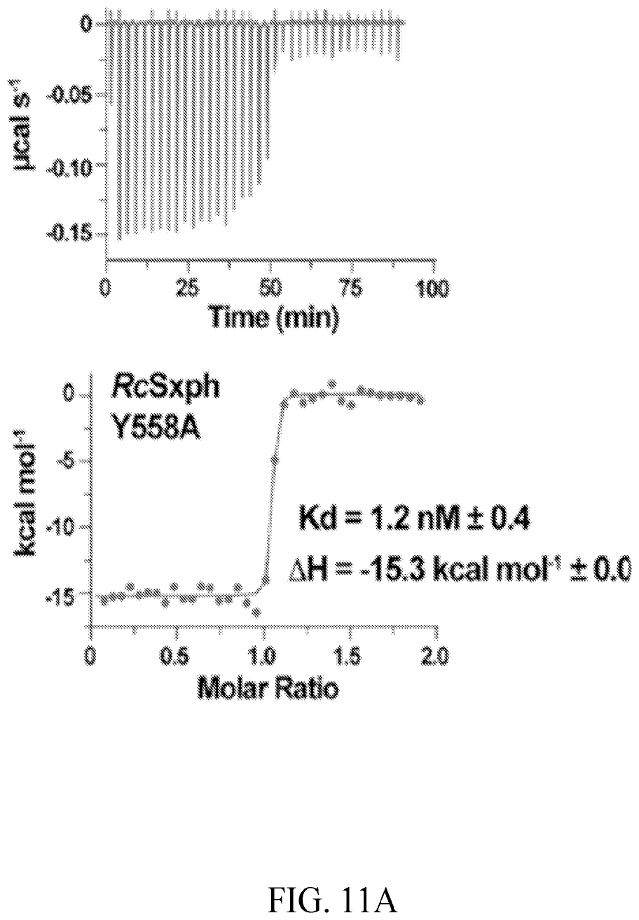

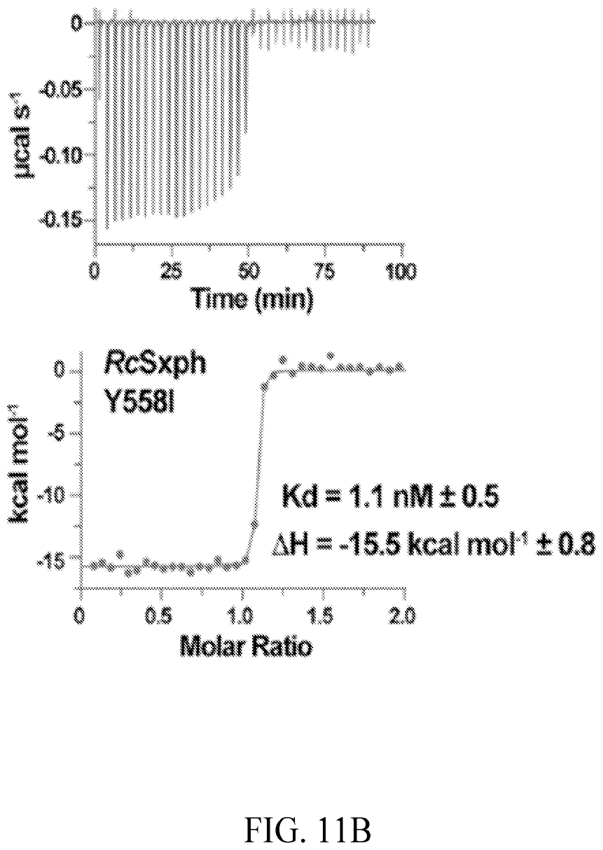

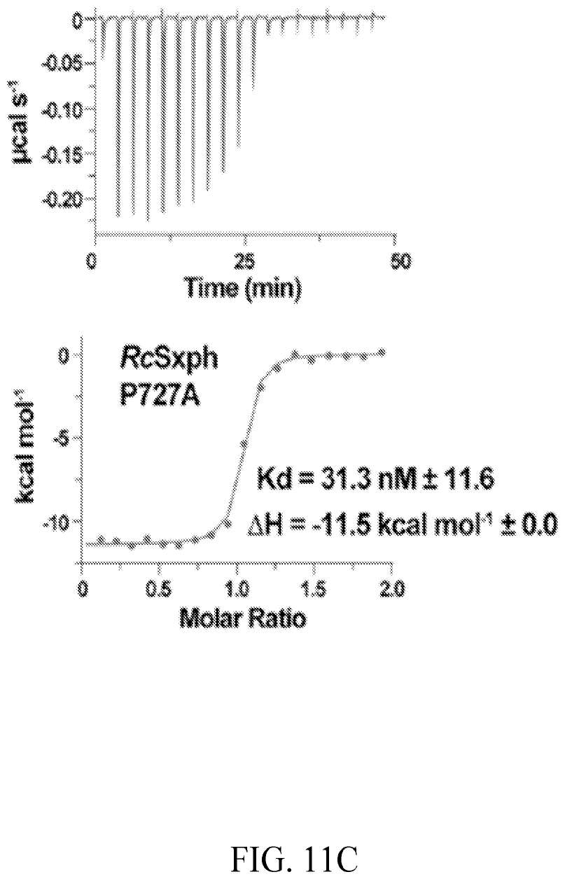

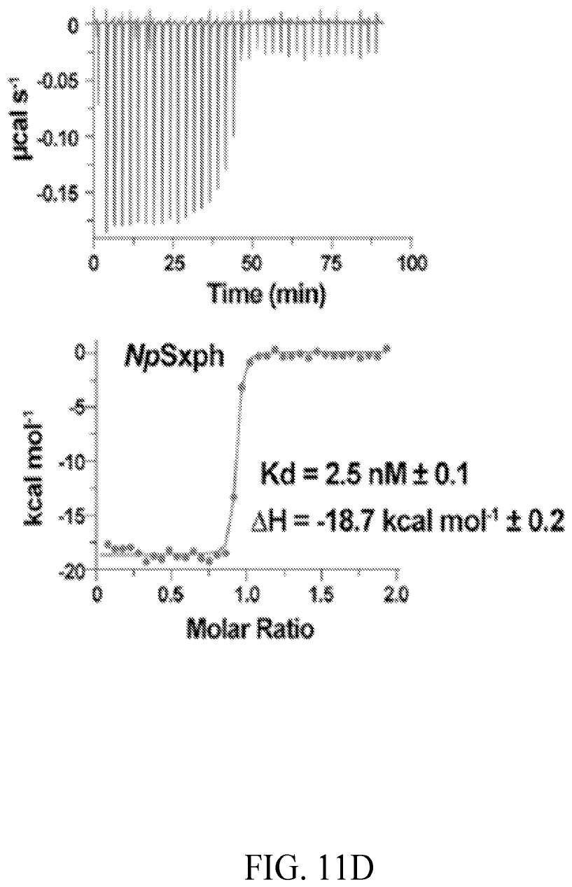

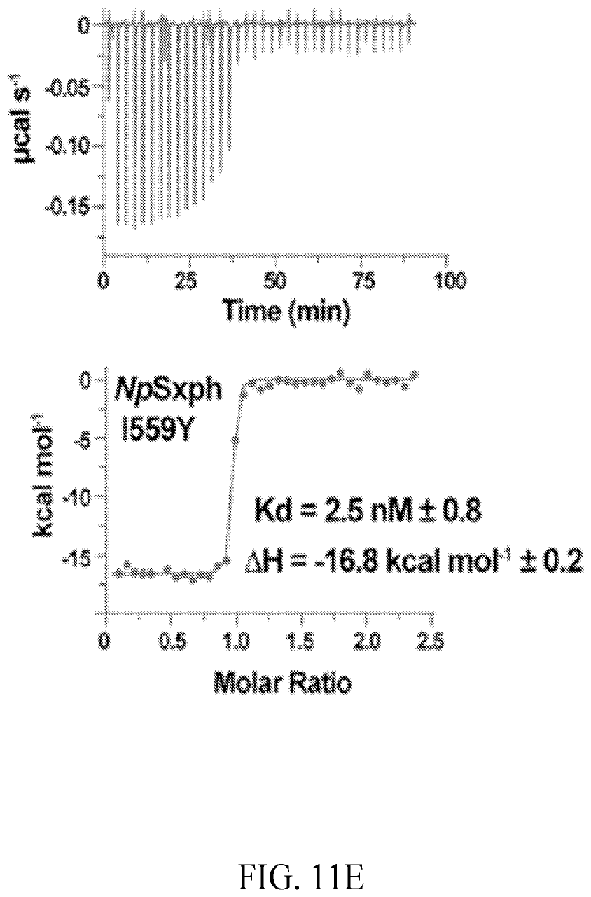

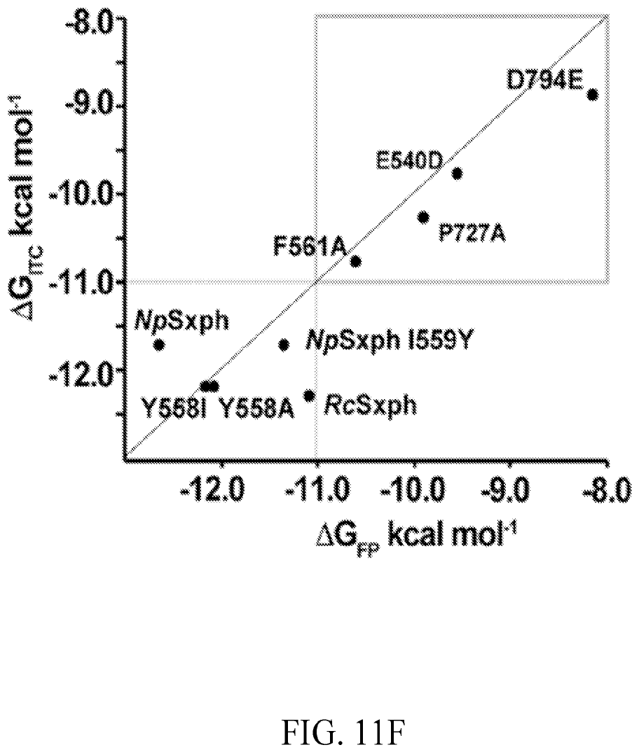

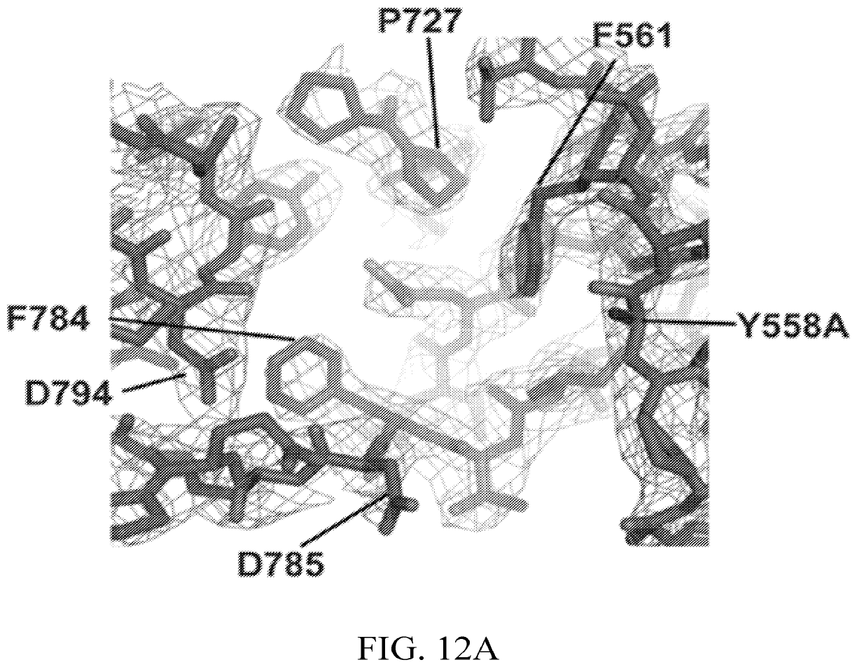

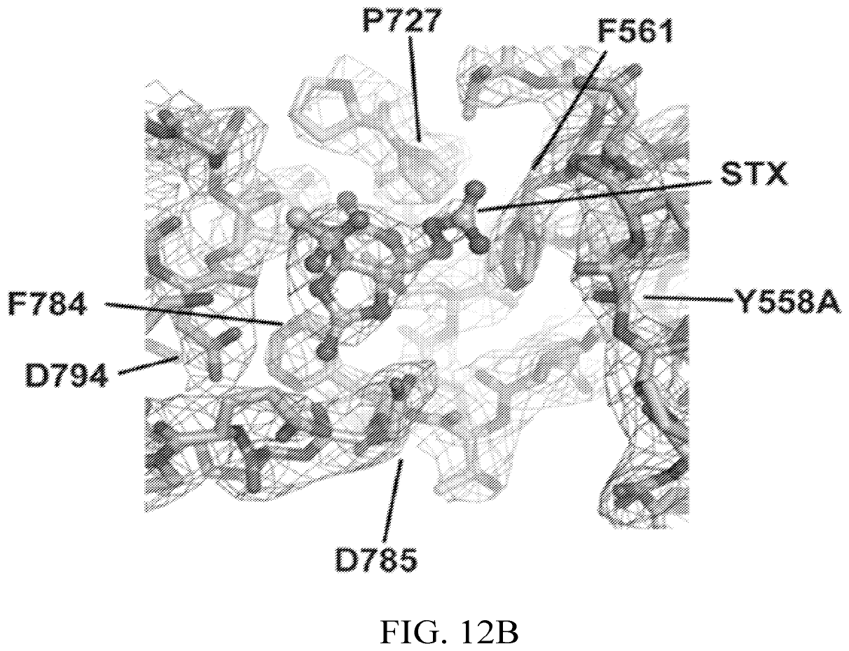

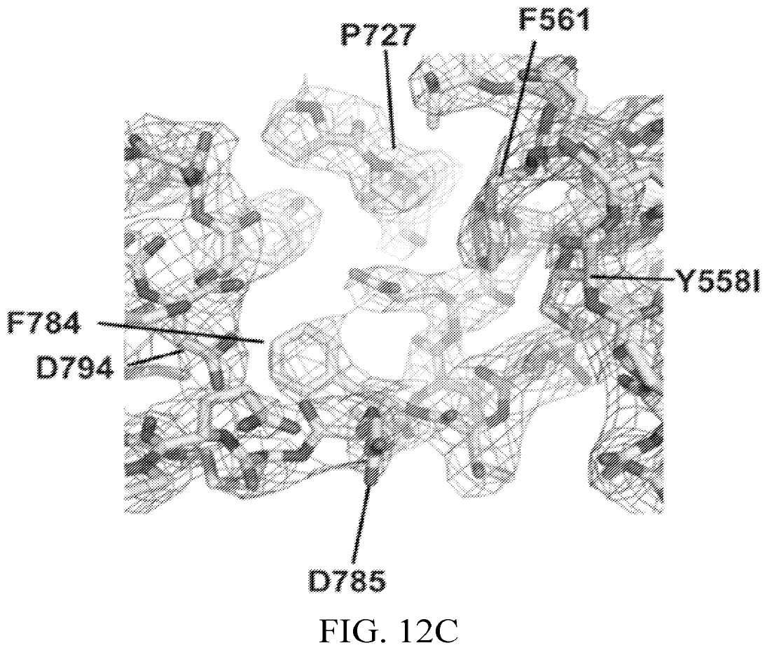

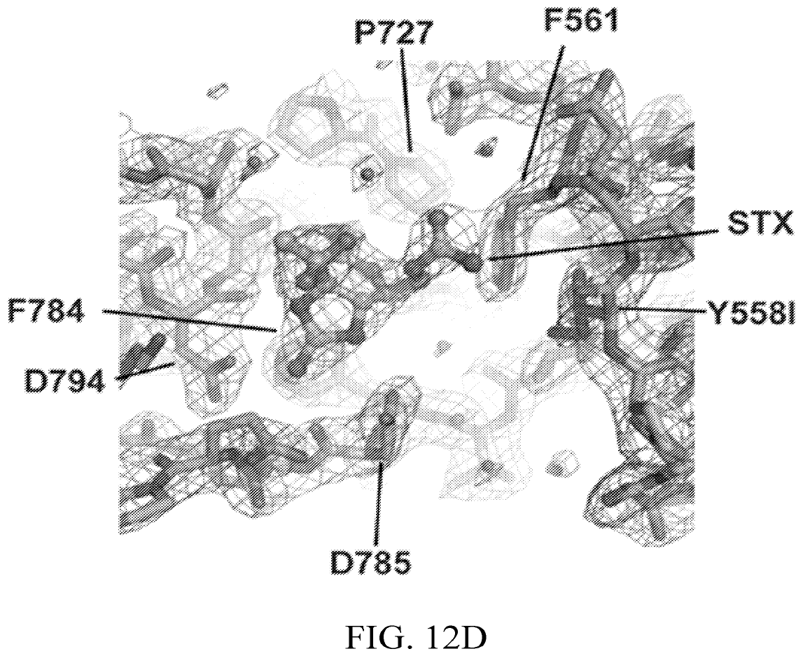

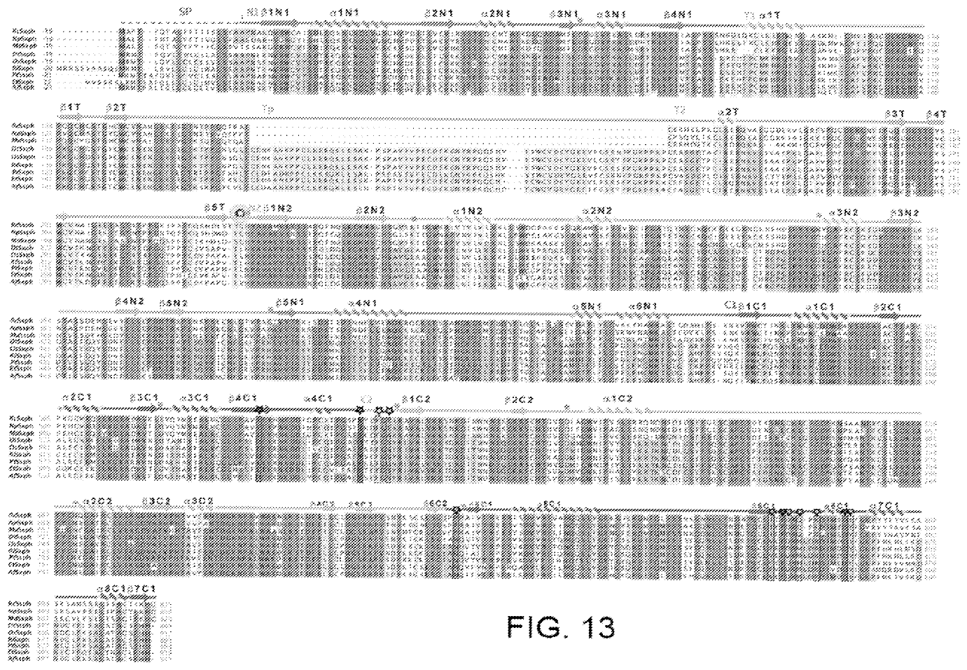

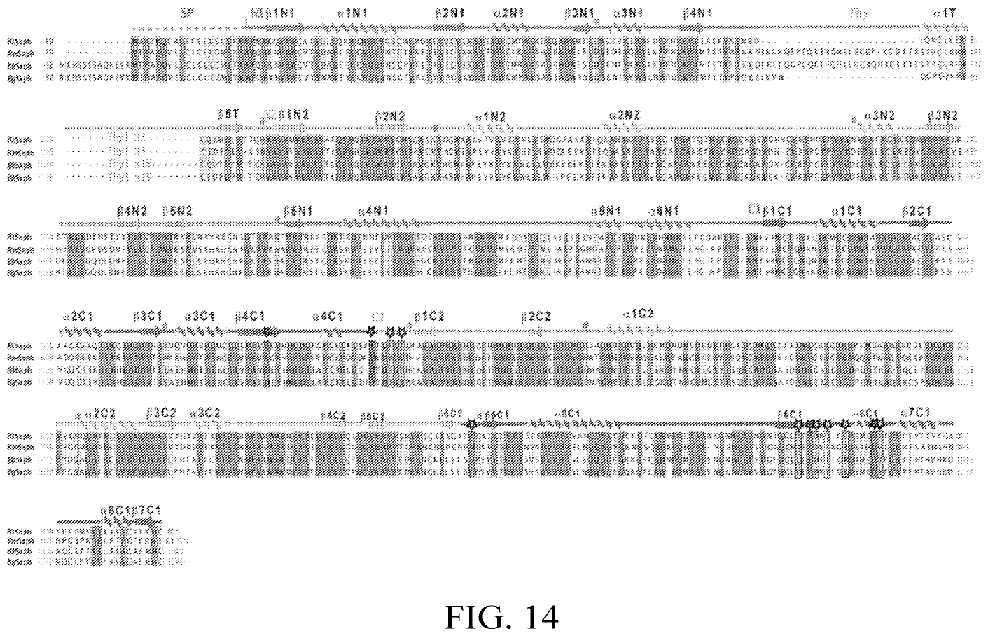

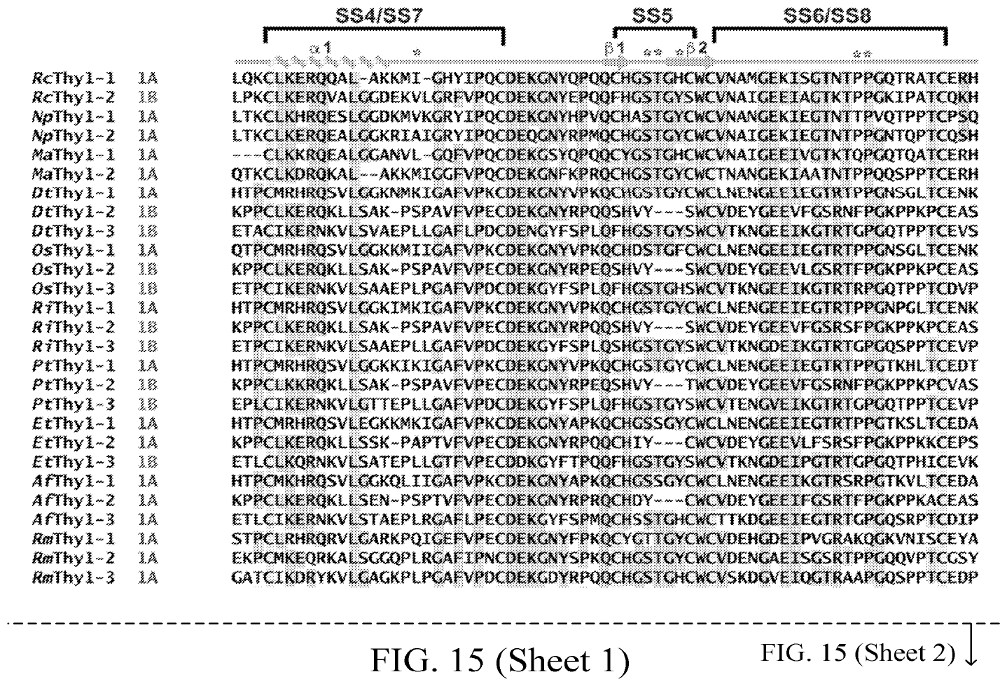

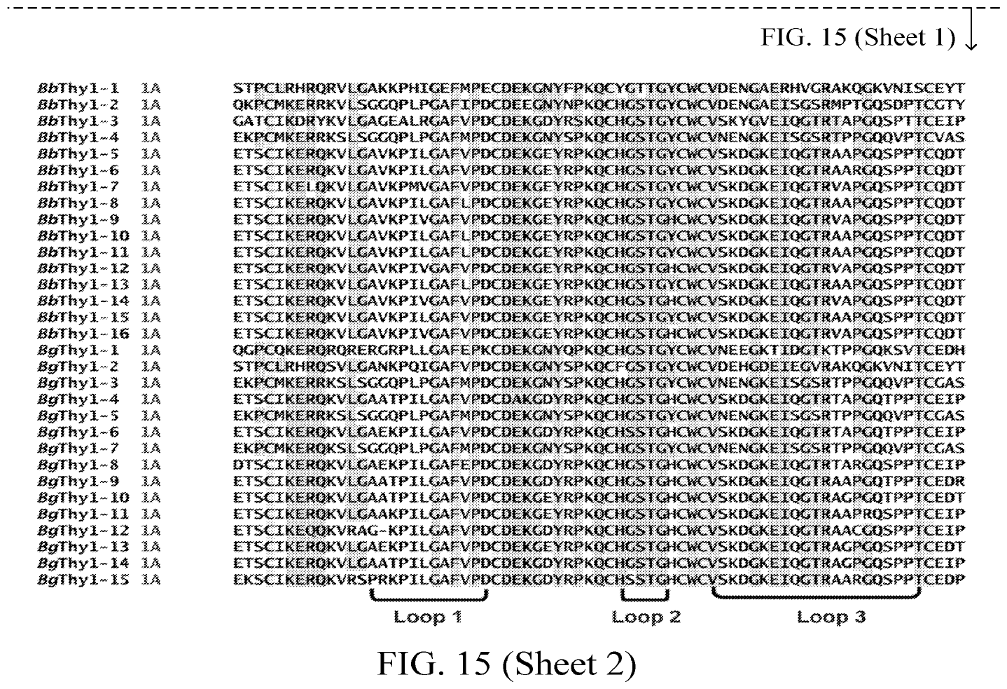

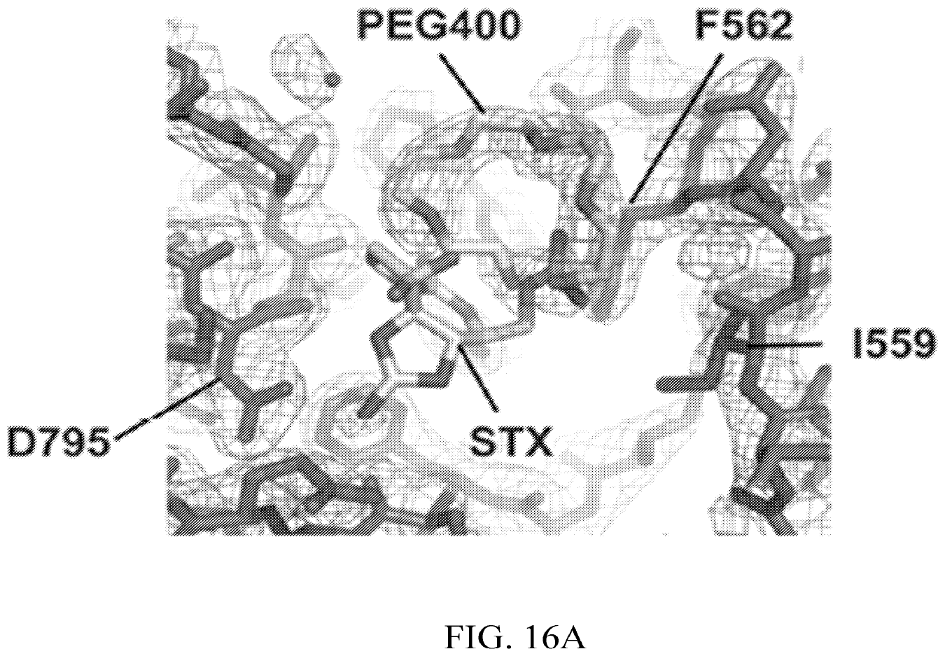

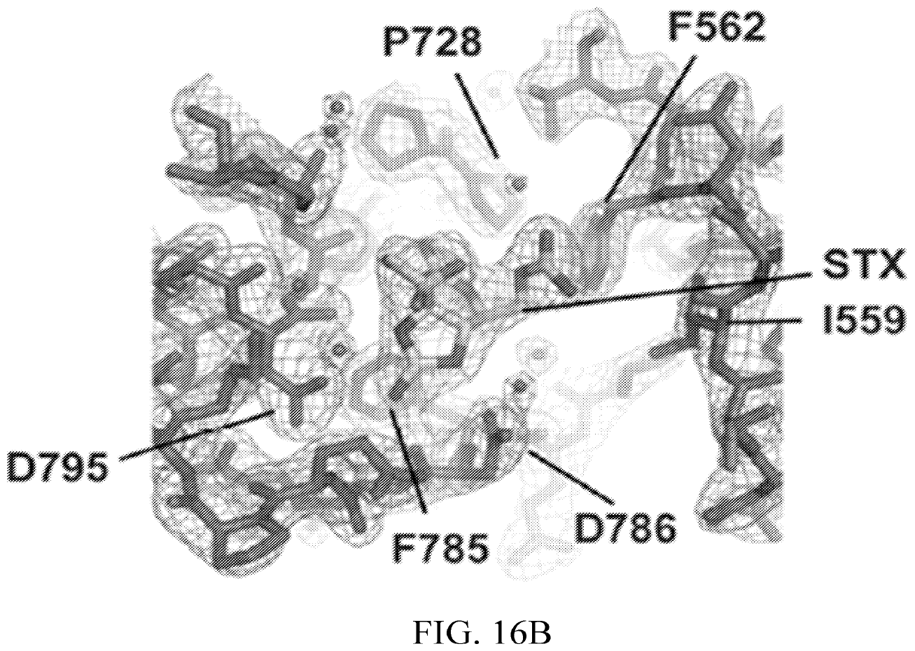

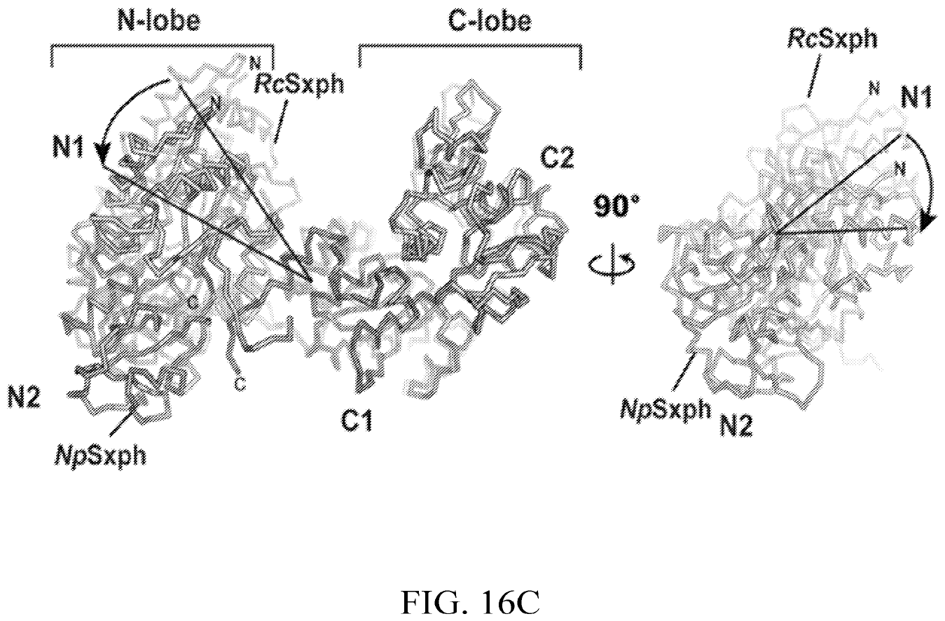

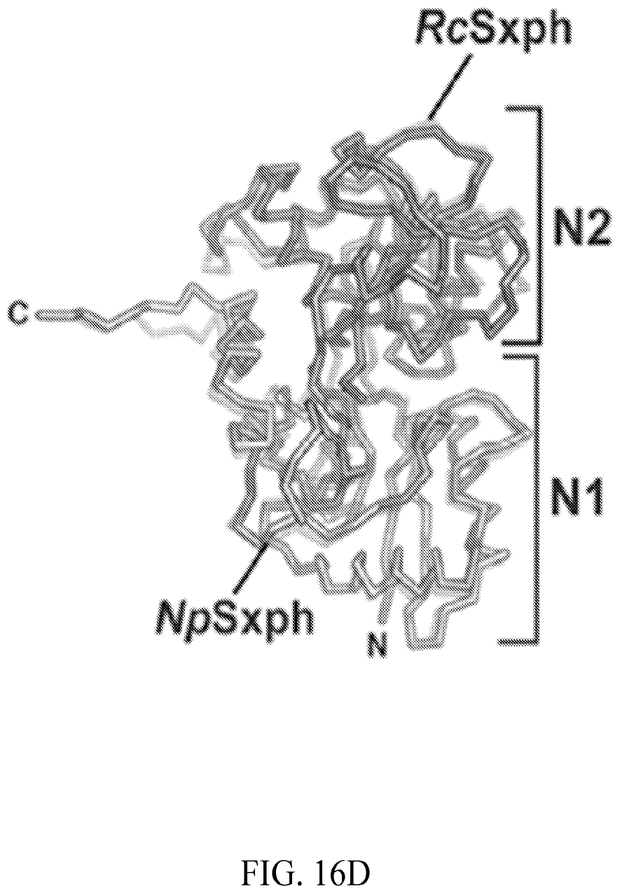

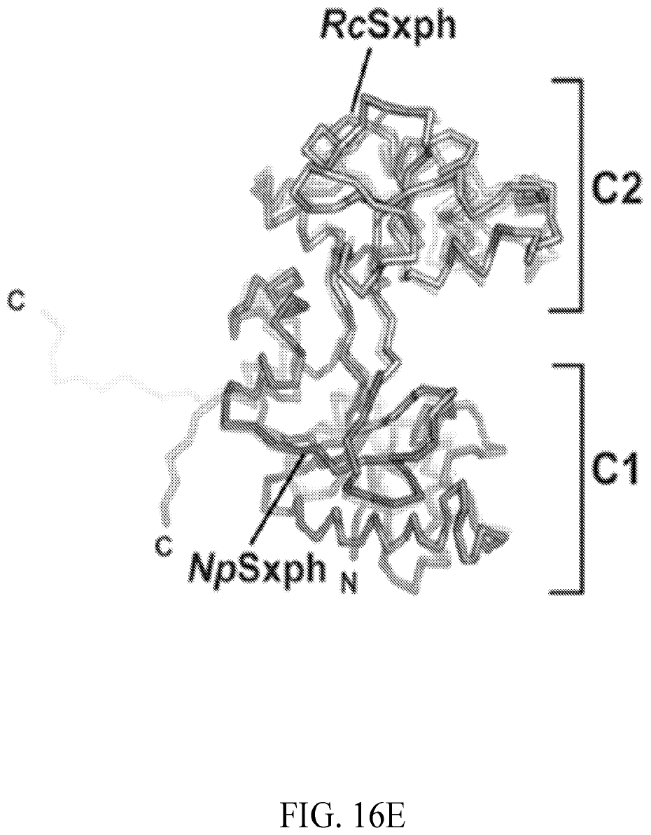

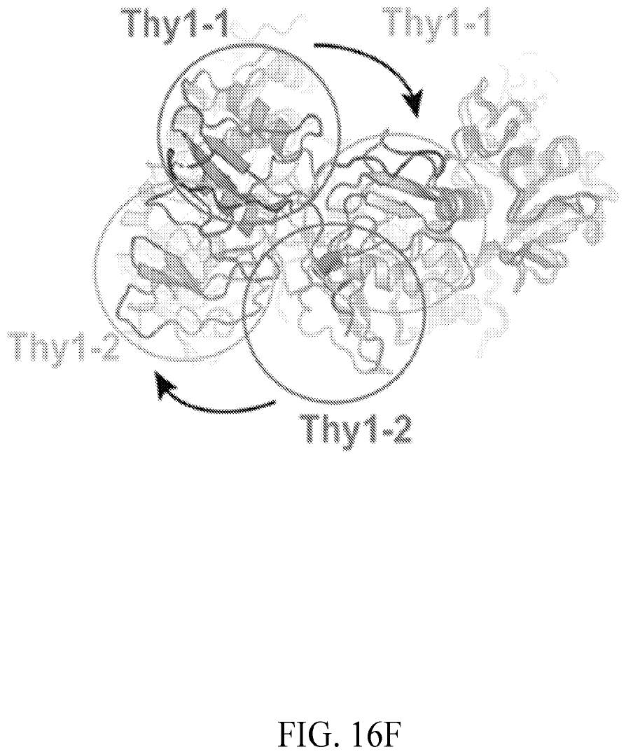

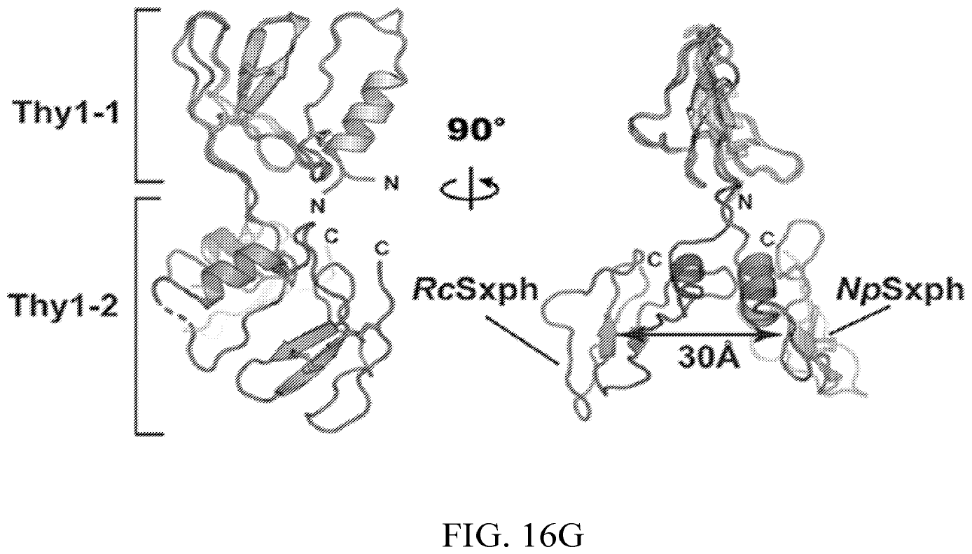

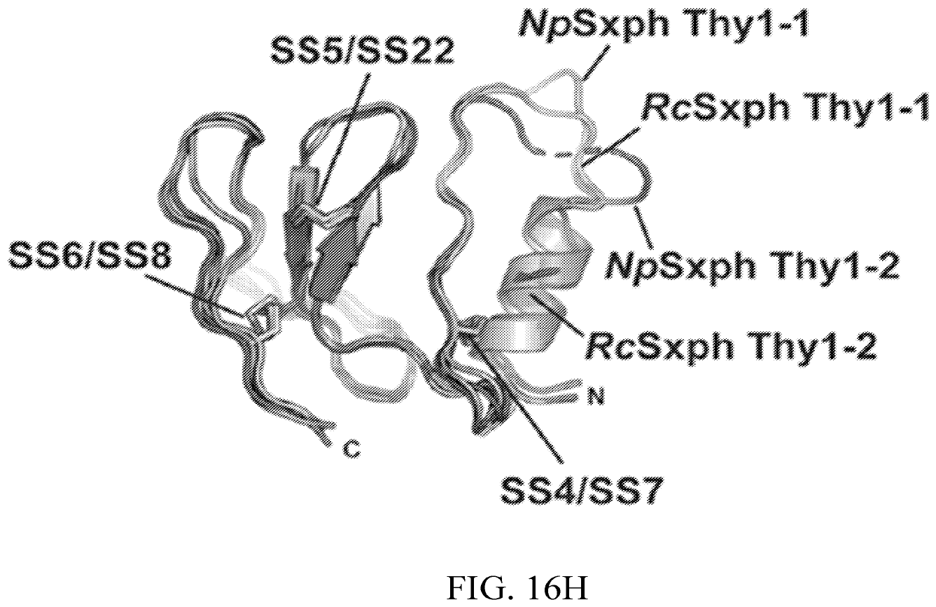

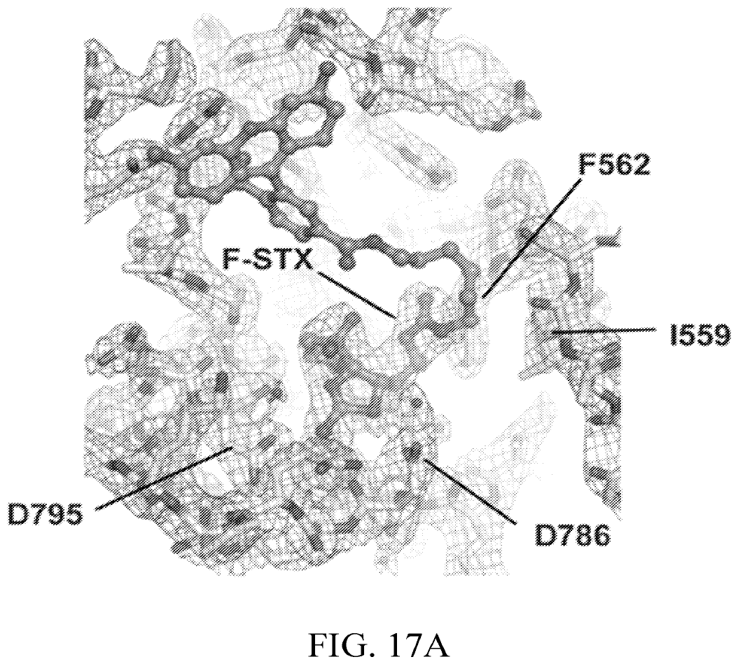

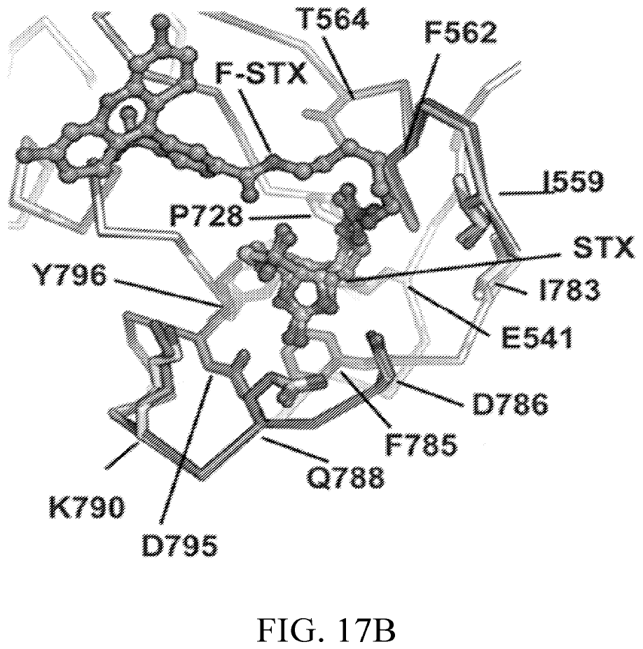

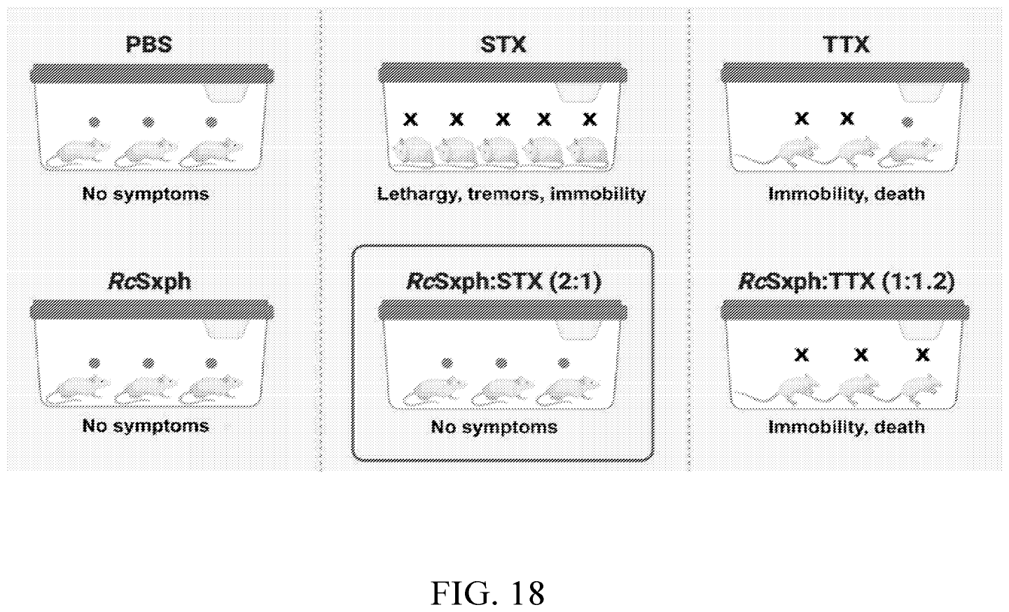

We crystallized and determined the structure of NpSxph, alone and co-crystallized with STX to compare STX binding modes among Sxph family members. NpSxph and STX:NpSxph crystals diffracted X-rays to resolutions of 2.2 Å and 2.0 Å, respectively, and were solved by molecular replacement (FIGS. 6A, S10A and B). As expected from the similarity to RcSxph, NpSxph is built on a transferrin fold (FIG. 6A) and has the same 21 disulfides found in RcSxph, as well as an additional 22^{nd} disulfide in the Type 1A thyroglobulin domain of NpSxph Thy1-2. However, structural comparison of NpSxph and RcSxph reveals a number of unexpected large-scale domain rearrangements.

The NpSxph N-lobe is displaced along the plane of the molecule by ˜30° and rotated around the central axis by a similar amount (FIG. S10C). NpSxph N-lobe and C-lobe lack Fe³⁺ binding sites (FIG. 5A), and despite the N-lobe displacement relative to RcSxph adopt closed and open conformations, respectively as in RcSxph (Yen et al.) (FIG. S10D-E) (RMSD_{Ca}=1.160 Å and 1.373 Å for NpSxph and RcSxph N- and C-lobes, respectively). Surprisingly, the two NpSxph Thy1 domains are in different positions than in RcSxph and appear to move as a unit by ˜90° with respect to the central transferrin scaffold (FIG. S10F and Supplementary movie M3) and a translation of ˜30 Å of Thy1-2 (FIG. S10G). Thy1-1 is displaced from a site over the N-lobe in RcSxph to one in which it interacts with the NpSxph C-lobe C2 subdomain and Thy1-2 moves from between the N and C-lobes in RcSxph where it interacts with the C1 subdomain, to a position in NpSxph where it interacts with both N-lobe subdomains. Consequently, the interaction between the C-lobe b-strand b7C1 and Thy1-2 b5 observed in RcSxph is absent in NpSxph. Despite these domain-scale differences, Thy1-1 and Thy1-2 are structurally similar to each other (RMSD_{Ca}=1.056 Å) and to their RcSxph counterparts (FIG. S10H) (RMSD_{Ca}=1.107 Å and 0.837, respectively). Further, none of these large scale changes impact the STX binding site, which is found on the C1 domain as in RcSxph (FIG. 5A).

Comparison of the apo- and STX-bound NpSxph structures shows that there are essentially no STX binding site conformational changes upon STX engagement, apart from the movement of Asp786 to interact with the STX five-membered guanidinium ring (FIG. 6B and Supplementary Movie M4). This conformational change is shared with RcSxph (Yen et al.) and appears to be a common element of Sxph binding to STX. The movements of Tyr558 and its loop away from the STX binding site observed in RcSxph (Yen et al.) are largely absent in NpSxph for the Tyr558 equivalent position (Ile559) and its supporting loop. Hence, the NpSxph STX binding site is better organized to accommodate STX (FIG. 6B), similar to RcSxph Y558I (FIG. 3B). We also noted an electron density in the apo-NpSxph STX binding site that we assigned as a PEG400 molecule from the crystallization solution (FIG. S10A). This density occupies a site different from STX and is not present in the STX-bound complex (FIG. S10B) and suggests that other molecules may be able to bind the STX binding pocket.

We also determined the structure of an NpSxph:F-STX complex at 2.2 Å resolution (Table S1). This structure shows no density for the fluorescein moiety and has an identical STX pose to the NpSxph:STX complex (FIG. S11), providing further evidence that fluorescein does not interact with Sxph (cf. FIG. S3) even though it is tethered to the STX binding pocket and the FP assay faithfully reports on STX:Sxph interactions. Comparison of the NpSxph and RcSpxh STX poses shows essentially identical interactions with the tricyclic bis-guanidium core and reveals that the carbamate is able to occupy the pocket opened by the Y→I variant (FIG. 6C), as observed in RcSxph Y558I (FIG. 3B). This change, together with the more rigid nature of the NpSxph STX binding pocket likely contributes to the higher affinity of NpSxph for STX relative to RcSxph (Table 1). Taken together, the various structures of Sxph:STX complexes show how subtle changes, particularly at the Tyr558 position can influence STX binding and underscore that knowledge of the STX binding code can be used to tune the STX binding properties of different Sxphs.

### 2. Discussion

Our biochemical and structural characterization of a set of RcSxph mutants and Sxphs from diverse anurans reveals a conserved STX recognition code centered around six amino acid residues comprising two triads. One triad engages the STX bis-guanidinium core using carboxylate groups that coordinate each ring (RcSxph Glu540 and Asp794) and an aromatic residue that makes a cation-p interaction (RcSxph Phe784) with the STX concave face. This recognition motif is shared with Na_{V}s, the primary target of STX in PSP (Thomas-Tran and DuBois, Shen et al. 2018, Shen et al. 2019) (FIG. 2C and D) and showcases a remarkably convergent STX recognition strategy. The second amino acid triad (RcSxph residues Tyr558, Phe561, and Pro727) largely interacts with the carbamate moiety and contains a site, Tyr558 and its supporting residue Ile782, where amino acid changes, including those found in some Anuran Sxphs (FIG. 5), enhance STX binding. Structural studies of RcSxph mutants and the High Himalaya Frog NpSxph show that STX-affinity enhancing changes in this region of the binding site act by reducing the degree of conformational change associated with STX binding (FIGS. 3 and 6 C-D). These findings reveal one strategy for creating high affinity STX binding sites. Importantly, enhancing the affinity of Sxph for STX through changes at either site increases the capacity of RcSxph to rescue Na_{V}s from STX block (FIG. 4) and demonstrates that an understanding of the STX recognition code enables rational modification of Sxph binding properties. Thus, exploiting the information in the STX recognition code defined here should enable design of Sxphs as STX sensors or agents for treating STX poisoning.

Although STX binding activity has been reported in a variety of diverse invertebrates (Llewellyn et al.) and vertebrates (Llewellyn et al., Tanaka et al.), only two types of STX binding proteins have been identified and validated, Sxphs from frogs (Mahar et al., Abderemane-Ali et al.) and the STX and TTX binding proteins from pufferfish (Yotsu et al. 2001, Yotsu et al. 2010). Our discovery of a set of ten new Sxphs that bind STX with high affinity (FIGS. 5, S7, and S8) represents a substantial expansion of the Sxph family and reveals diverse natural variation of residues that are important for STX binding (FIG. 5B). Most notably, E540D, a change that compromises STX binding in RcSxph by ˜14-fold, occurs in five of the newly identified Sxphs. Nevertheless, functional studies show that RiSxph, which bears an Asp at this site, binds STX more strongly than RcSxph (Table 2). Hence, the natural variations at other RiSxph STX binding pocket sites must provide compensatory interactions to maintain a high STX binding affinity. Understanding how such variations impact STX engagement or influence the capacity of these proteins to discriminate among STX congeners (Llewellyn et al.) remain important unanswered questions. The striking abundance of Sxphs in diverse amphibians, representing linages separated by ˜140 million years (Feng et al.), that are not known to carry STX raises intriguing questions regarding the selective pressures that have caused these disparate amphibians to maintain this STX binding protein.

Besides the conserved STX binding site, all of the amphibian Sxphs possess a set of Thy1 domains similar to those in RcSxph that are known to act as protease inhibitors (Lenarčič et al.). The discovery of Sxphs in diverse Anurans shows that these domains are a common feature of the Sxph family and occur in varied numbers. The expansion of this insertion into the Sxph scaffold of 15-16 Thy1 domains in toad Sxphs is particularly striking. Structural comparisons between RcSxph and NpSxph, both of which have two Thy1 domains (FIG. 5A), show that these domains can adopt different positions with respect to the shared transferrin core (FIG. S10F). Whether these Thy1 domains are important for Sxph-mediated toxin resistance mechanisms (Mahar et al., Abderemane-Ali et al.) or serve some other function and whether the diversity of Thy1 repeats impacts function remains unknown. Our definition of Sxph STX binding code, which provides a guide for deciphering variation in the Sxph STX binding site (FIG. 5B), and high variability in Thy1 repeats among Anuran Sxphs should provide a guide for finding other Sxphs within this widespread and diverse family of amphibians family.

STX interacts with a variety of target proteins including select Na_{V} isoforms (Duran-Riveroll and Cembella) and other channels (Su et al., Wang et al.), diverse soluble STX binding proteins (Mahar et al., Yen et al., Yotsu et al. 2001, Yotsu et al. 2010, Takati et al., Lin et al.), and some enzymes (Llewellyn, Lukowski et al. 2019, Lukowski et al. 2020). The identification of the Sxph STX recognition code together with the substantial expansion of the Sxph family provide a foundation for developing a deeper understanding of the factors that enable proteins to bind STX with high affinity. Exploration of such factors should be facilitated by the TF, FP, and ITC assays established here that enable Sxph:STX interactions to be probed using a range of samples quantities (TF: 600 ng STX, 25 μg Sxph; FP: 1 ng F-STX, 3 μg Sxph; ITC: 5 μg STX, 300 μg Sxph). In cases of limited samples, such as difficult to obtain STX congeners, the excellent agreement among the assays should provide a reliable basis for interpretation of binding properties. These assays, together with delineation of the Sxph STX binding code, together with the expansion of the Sxph family here provide a framework for understanding the lethal effects of this potent neurotoxin and ‘toxin sponge’ STX resistance mechanisms (Mahar et al., Abderemane-Ali et al.). This knowledge and may enable the design of novel PSP toxin sensors and agents that could mitigate STX intoxication.

### 3. Materials and Methods

### Expression and Purification of Sxphs and Mutants

R. catesbeiana Sxph (RcSxph) and mutants were expressed using a previously described RcSxph baculovirus expression system in which RcSxph carries in series, a C-terminal 3C protease cleavage site, green fluorescent protein (GFP), and a His₁₀ tag (Yen et al.). The gene encoding Nanorana parkeri Sxph (NpSxph) including its N-terminal secretory sequence (GenBank: XM_018555331.1) was synthesized and subcloned into a pFastBac1 vector using NotI and XhoI restriction enzymes by GenScript and bears the same C-terminal tags as RcSxph. RcSxph and NpSxph mutants were generated using the QuikChange site-directed mutagenesis kit (Stratagene). All constructs were sequenced completely. RcSxph, RcSxph mutants, NpSxph and NpSxph I559Y were expressed in Spodoptera frugiperda (Sf9) cells using a baculovirus expression system as described previously for RcSxph (Yen et al.) and purified using a final size exclusion chromatography (SEC) run in 150 mM NaCl, 10 mM HEPES, pH 7.4. Protein concentrations were determined by measuring UV absorbance at 280 nm using the following extinction coefficients calculated using the ExPASY server (https://web.expasy.org/protparam/): RcSxph Y558 mutants, 94,875 M⁻¹ cm⁻¹; RcSxph F784C 96,490 M⁻¹ cm⁻¹; RcSxph F784Y 97,855 M⁻¹ cm⁻¹, RcSxph and all other RcSxph mutants 96,365 M⁻¹ cm⁻¹; NpSxph 108,980 M⁻¹ cm⁻¹; and NpSxph I559Y 110,470 M⁻¹ cm⁻¹.

### Thermofluor (TF) Assay of Toxin Binding

Thermofluor assays for STX and TTX binding were developed as outlined (Huynh and Partch). TTX was purchased from Abcam (Catalog #ab120054). 20 μL samples containing 1.1 μM RcSxph, NpSxph, or mutants thereof, 5× SYPRO Orange dye (Sigma-Aldrich, S5692, stock concentration 5000×), 0-20 μM STX or TTX, 150 mM NaCl, 10 mM HEPES, pH 7.4 were set up in 96-well PCR plates (Bio-Rad), sealed with a microseal B adhesive sealer (Bio-Rad) and centrifuged (1 min, 230×g) prior to thermal denaturation. The real-time measurement of fluorescence using the HEX channel (excitation 515-535 nm, emission 560-580 nm) was performed in CFX Connect Thermal Cycler (Bio-Rad). Samples were heated from 25° C. to 95° C. at 0.2° C. min⁻¹. Melting temperature (Tm) was calculated by fitting the denaturation curves using a Boltzmann sigmoidal function and GraphPad Prism: F=Fₘᵢₙ+(Fₘₐₓ−Fₘᵢₙ)/(1+exp((Tm−T)/C)), where F is the fluorescence intensity at temperature T, Fₘᵢₙ and Fₘₐₓ are the fluorescence intensities before and after the denaturation transition, respectively, Tm is the midpoint temperature of the thermal unfolding transition, and C is the slope at Tm (Huynh and Partch). □Tm=Tm_{Sxph+20 μM toxin}−Tm_{Sxph}.

### Fluorescence Polarization Assay

Fluorescence polarization assays were performed as described (Rossi and Taylor). 100 μL samples containing 1 nM fluorescein labeled STX (F-STX), 150 mM NaCl, 10 mM HEPES, pH 7.4, and Sxph variants at the following concentration ranges (RcSxph and RcSxph T563A, I782A, F784Y, D785N, Q787A, Q787E, K789A, and Y795A, 0-75 nM; RcSxph Y558A and I782A/Y558A, 0-24 nM; RcSxph Y558I, 0-37.5 nM; RcSxph Y558F, 0-100 nM; RcSxph I782F 0-150 nM; RcSxph F561A, 0-300 nM; RcSxph F784L, 0-500 nM; RcSxph E540D, P727A, and D785A, 0-600 nM; RcSxph F784A, 0-4.8 μM; RcSxph E540A and F784C, 0-10 μM; RcSxph D794A and D794E, 0-12.5 μM; RcSxph F784S, 0-17 μM; RcSxph D794N, 0-20 μM; RcSxph E540Q, 0-25 μM; NpSxph and NpSxph I559Y, 0-75 nM) were prepared in 96-well black flat-bottomed polystyrene microplates (Greiner Bio-One) and sealed with an aluminum foil sealing film (AlumaSeal II), and incubated at room temperature for 0.5 h before measurement. Measurements were performed at 25° C. on a Synergy H1 microplate reader (BioTek) using the polarization filter setting (excitation 485 nm, emission 528 nm). Binding curves for representative high affinity (RcSxph, NpSxph, and RcSxph-Y558I) and low affinity (RcSxph-E540D) proteins were compared at 0.5 h, 1.5 h, 4.5 h, and 24 h, post mixing and indicated that equilibrium was reached by 0.5 h for all samples. The dissociation constants were calculated using GraphPad Prism by fitting fluorescence millipolarization (mP=P·10⁻³, where P is polarization) as a function of Sxph concentration using the equation: P={(P_{bound}−P_{free}) [Sxph]/(K_{d}+[Sxph])}+P_{free}, where P is the polarization measured at a given Sxph concentration, P_{free} is the polarization of Sxph in the absence of F-STX, and P_{bound} is the maximum polarization of Sxph bound by F-STX (Rossi and Taylor, Hansen et al.).

### Isothermal Titration Calorimetry (ITC)

ITC measurements were performed at 25° C. using a MicroCal PEAQ-ITC calorimeter (Malvern Panalytical). RcSxph, RcSxph mutants, NpSxph, and NpSxph I559Y were purified using a final size exclusion chromatography step in 150 mM NaCl, 10 mM HEPES, pH 7.4. 1 mM STX stock solution was prepared by dissolving STX powder in MilliQ water. This STX stock was diluted with the SEC buffer to prepare 100 μM or 300 μM STX solutions having a final buffer composition of 135 mM NaCl, 9 mM HEPES, pH 7.4. To match buffers between the Sxph and STX solutions, the purified protein samples were diluted with MilliQ water to reach a buffer concentration of 135 mM NaCl, 9 mM HEPES, pH 7.4. (30 μM for RcSxph D794E; 10 μM for RcSxph, other RcSxph mutants, NpSxph, and NpSxph I559Y) Protein samples were filtered through a 0.22 μm spin filter (Millipore) before loading into the sample cell and titrated with STX (300 μM STX for RcSxph D794 and 100 μM STX for RcSxph, other RcSxph mutants, NpSxph, and NpSxph I559Y) using a schedule of 0.4 μL titrant injection followed by 35 injections of 1 μL for the strong binders (RcSxph, RcSxph Y558I, RcSxph Y558A, RcSxph F561A, NpSxph, and NpSxph I559Y) and a schedule of 0.4 μL titrant injection followed by 18 injections of 2 μL for the weak binders (RcSxph P727A, RcSxph E540D, and RcSxph D794E). The calorimetric experiment settings were: reference power, 5 μcal/s; spacing between injections, 150 s; stir speed 750 rpm; and feedback mode, high. Data were analyzed using MicroCal PEAQ-ITC Analysis Software (Malvern Panalytical) using a single binding site model. The heat of dilution from titrations of 100 μM STX in 135 mM NaCl, 9 mM HEPES, pH 7.4 into 135 mM NaCl, 9 mM HEPES, pH 7.4 was subtracted from each experiment to correct the baseline.

### Crystallization, Structure Determination, and Refinement

RcSxph mutants were crystallized at 4° C. as previously described for RcSxph (Yen et al.). Briefly, purified protein was exchanged into a buffer of 10 mM NaCl, 10 mM HEPES, pH 7.4 and concentrated to 65 mg ml⁻ using a 50-kDa cutoff Amicon Ultra centrifugal filter unit (Millipore). Crystallization was set up by hanging drop vapor diffusion using a 24-well VDX plate with sealant (Hampton Research) using 3 μL drops having a 2:1 (v:v) ratio of protein:precipitant. For co-crystallization with STX, STX and the target RcSxph mutants were mixed in a molar ratio of 1.1:1 STX:Sxph and incubated on ice for 1 hour before setting up crystallization. RcSxph-Y558I and RcSxph-Y558I:STX were crystallized from solutions containing 27% 2-methyl-2,4-pentanediol, 5% PEG 8000, 0.08-0.2 M sodium cacodylate, pH 6.5. RcSxph-Y558A and RcSxph-Y558A:STX were crystallized from solutions containing 33% 2-methyl-2,4-pentanediol, 5% PEG 8000, 0.08-0.2 M sodium cacodylate, pH 6.5. To obtain crystals of the RcSxph:F-STX complex, RcSxph was crystallized from solutions containing 33% 2methyl-2,4-pentanediol, 5% PEG 8000, 0.11-0.2 M sodium cacodylate, pH 6.5 and then soaked with F-STX (final concentration, 1 mM) for 5 hours before freezing.

For NpSxph crystallization, protein was purified as described for RcSxph, except that the final size exclusion chromatography was done using 30 mM NaCl, 10 mM HEPES, pH 7.4. Protein was concentrated to 30-40 mg ml⁻ using a 50-kDa cutoff Amicon Ultra centrifugal filter unit (Millipore). NpSxph crystals were obtained by hanging drop vapor diffusion at 4° C. using 1:1 v/v ratio of protein and precipitant. NpSxph crystals were obtained from 400 nl drops set with Mosquito crystal (Sptlabtech) using 20-25% (v/v) PEG 400, 4-5% (w/v) PGA-LM, 100-200 mM sodium acetate, pH 5.0. For STX co-crystallization, NpSxph and STX (5 mM stock solution prepared in MilliQ water) were mixed in a molar ratio of 1.2:1 STX:NpSxph and incubated on ice for 1 hour before setting up the crystallization trays. Crystals of the STX:NpSxph were grown in the same crystallization solution as NpSxph. NpSxph and NpSxph:STX crystals were harvested and flash-frozen in liquid nitrogen without additional cryoprotectant.

X-ray datasets for RcSxph mutants, RcSxph mutant:STX complexes, RcSxph: F-STX, NpSxph, and NpSxph:STX were collected at 100K at the Advanced Photon Source (APS) beamline 23 ID B of Argonne National Laboratory (Lemont, IL), processed with XDS (Kabsch, 2010) and scaled and merged with Aimless (Evans and Murshudov). RcSxph structures were determined by molecular replacement of RcSxph chain B from (PDB: 6O0F) using Phaser from PHENIX (Adams et al.). The resulting electron density map was thereafter improved by rigid body refinement using phenix.refine. The electron density map obtained from rigid body refinement was manually checked and rebuilt in COOT (Emsley and Cowtan) and subsequent refinement was performed using phenix.refine.

The NpSxph structure was solved by molecular replacement using the MoRDa pipeline implemented in the Auto-Rikshaw, automated crystal structure determination platform (Panjikar et al.). The scaled X-ray data and amino-acid sequence of NpSxph were provided as inputs. The molecular replacement search model was identified using the MoRDa domain database derived from the Protein Data Bank (PDB). The MR solution was refined with REFMAC5 (N. Collaborative Computational Project), density modification was performed using PIRATE (Cowtan 2000, Winn et al.), and was followed by the automated model building in BUCCANEER (Cowtan 2006, Cowtan 2008). The partial model was further refined using REFMAC5 and phenix.refine. Dual fragment phasing was performed using OASIS-2006 (Winn et al.) based on the automatically refined model, and the resulting phases were further improved in PIRATE. The next round of model building was continued in ARP/wARP (Morris et al.) and the resulting structure was refined in REFMAC5. The final model generated in Auto-Rikshaw (720 out of 825 residues built, and 625 residues automatically docked) was further used as a MR search model in Phaser from PHENIX (Adams et al.). The quality of the electron density maps allowed an unambiguous assignment of most of the amino acid residues with the exception of the loop regions and the C2 subdomain showing poor electron density. The apo-NpSxph structure was completed by manual model building in COOT (Emsley and Cowtan) and multiple rounds of refinement in phenix.refine. The NpSxph:STX: structure was solved by molecular replacement using the NpSxph structure as a search model in Phaser from PHENIX (Adams et al.). After multiple cycles of manual model rebuilding in COOT (Emsley and Cowtan), iterative refinement was performed using phenix.refine. The quality of all models was assessed using MolProbity (Williams et al.) and refinement statistics.

RNA Sequencing of O. sylvatica, D. tinctorius, R. imitator, E. tricolor, A. femoralis, and M. aurantiaca Sxphs

Nearly all poison frog species were bred in the O'Connell Lab or purchased from the pet trade (Josh's Frogs) except for O. sylvatica, which was field collected as described in (McGugan et al.). De novo transcriptomes for O. sylvatica, D. tinctorius, R. imitator, E. tricolor, A. femoralis, and M. aurantiaca were constructed using different tissue combinations depending on the species. RNA extraction from tissues was performed using TRIzol™ Reagent (Thermo Fisher Scientific). Poly-adenylated RNA was isolated using the NEXTflex PolyA Bead kit (Bioo Scientific, Austin, USA) following manufacturer's instructions. RNA quality and lack of ribosomal RNA was confirmed using an Agilent 2100 Bioanalyzer or Tapestation (Agilent Technologies, Santa Clara, USA). Each RNA sequencing library was prepared using the NEXTflex Rapid RNAseq kit (Bioo Scientific). Libraries were quantified with quantitative PCR (NEBnext Library quantification kit, New England Biolabs, Ipswich, USA) and an Agilent Bioanalyzer High Sensitivity DNA chip, according to manufacturer's instructions. All libraries were pooled at equimolar amounts and were sequenced on four lanes of an Illumina HiSeq 4000 machine to obtain 150 bp paired-end reads. De novo transcriptomes were assembled using Trinity and once assembled were used to create a BLAST nucleotide database using the BLAST+ command line utilities. The amino acid Sxph sequence of L. catesbeiana was used as a query to tBLASTN against the reference transcriptome databases. The Sxph sequence for O. sylvatica was lacking the 5′ and 3′ ends, whose sequence was obtained using RACE as described above. After obtaining a full-length sequence, the top BLAST hits from each poison frog transcriptome were manually inspected and aligned to the O. sylvatica nucleotide sequence to find full sequences with high similarity. Either a single Sxph sequence from each transcriptome was found to be the best match, or there were multiple transcripts that aligned well, in which case a consensus alignment was created. The largest ORF from each species sequence was translated to create an amino acid sequence for alignment. For the D. tinctorius, R. imitator, and A. femoralis sequences, regions covering the STX binding site and transferrin-related iron-binding sites were confirmed by PCR and sanger sequencing.

Identification of P. terribilis, R. marina, B. bufo, and B. gargarizans Sxphs

All P. terribilis frogs were captive bred in the O'Connell lab poison frog colony. All were sexually mature individuals housed in 18×18×18-inch glass terraria, brought up on a diet of Drosophila melanogaster without additional toxins. Frogs were euthanized according to the laboratory collection protocol detailed by Fischer et al. 2019 and tissues were stored in RNALater. Eye tissue was rinsed in PBS before being placed into the beadbug tubes (Sigma-Aldrich, Z763756) prefilled with 1 mL TRIzol (Thermo Fisher Scientific, 15596018) and then RNA was extracted following manufacturer instructions. RNA was reverse transcribed into cDNA following the protocol outlined in Invitrogen's SuperScript IV Control Reactions First-Strand cDNA Synthesis reaction (Pub. no. MAN0013442, 16 Rev. B). After reverse transcription, cDNA concentration was checked via NanoDrop (Thermo Scientific, ND-ONE-W), and then aliquoted and stored at −20° C. until used for PCR. Saxiphilin was amplified from cDNA from P. terribilis in 50 μL polymerase chain reactions following the New England Biolabs protocol for Phusion® High-Fidelity PCR Master Mix with HF Buffer (30) (included DMSO). Each reaction was performed with 1 μL of cDNA. PCR primers were designed based on a O. sylvatica saxiphilin cDNA sequence previously generated by the O'Connell lab. PCR products were cleaned up using the Thermo Scientific GeneJET Gel Extraction and DNA Cleanup Micro Kit (Catalog number K0832) dimer removal protocol, and then sent out for Sanger Sequencing via the GeneWiz “Premix” service. The segments from sequencing were aligned and assembled but found that the 5′ and 3′ ends of the Sxph sequence for P. terribilis were missing, thus the 5′ and 3′ end sequences were subsequently obtained using RACE. 5′ and 3′-RACE-Ready cDNA templates were synthesized using a SMARTer® RACE 5′/3′ Kit (Takara Bio, USA) and subsequently used to amplify 5′ and 3′ end sequences of P. terribilis Sxph using internal gene specific primers.

Initial Sxph sequence for R. marina was obtained from the genome by searching the draft Cane Toad genome (Edwards et al.) with tBLASTN using the L. catesbeiana Sxph amino acid sequence as a query. Matching segments from the genome were pieced together to produce an amino acid sequence, however, this sequence was missing part of the 3′ end. To obtain the 3′ residues, the nucleotide sequences from the genome were used to design primers for 3′ Rapid Amplification of cDNA Ends (RACE). One R. marina individual from a lab-housed colony was thus euthanized in accordance with UCSF IACUC protocol AN136799, and a portion of the liver was harvested for total RNA extraction using TRIzol™ Reagent (Thermo Fisher Scientific). Total RNA integrity was assessed on a denaturing formaldehyde agarose gel. 3′-RACE-Ready cDNA template was synthesized using a SMARTer® RACE 5′/3′ Kit (Takara Bio, USA) and subsequently used to amplify 3′ end sequences of R. marina Sxph using internal gene specific primers designed from R. marina genomic sequences. 3′ end sequences of R. marina Sxph were determined by gel extraction using QIAquick Gel Extraction Kit (QIAGEN) and verified by sanger sequencing.

Sequences for Bufo (Common Toad) and Bufo gargarizans (Asiatic toad) Sxphs were identified as sequence searches (tBLASTN) using the RmSph sequence as a query.

### Two-Electrode Voltage Clamp Electrophysiology

Two-electrode voltage-clamp (TEVC) recordings were performed on defolliculated stage V-VI Xenopus laevis oocytes harvested under UCSF-IACUC protocol AN178461. Capped mRNA for P. terribilis (Pt) Na_{V}1.4 (GenBank: MZ545381.1) expressed in a pCDNA3.1 vector (Abderemane-Ali et al.) was made using the mMACHINE™ T7 Transcription Kit (Invitrogen). Xenopus oocytes were injected with 3-6 ng ofPtNa_{V}1.4 and TEVC experiments were performed 1-2 days post-injection. Data were acquired using a GeneClamp 500B amplifier (MDS Analytical Technologies) controlled by pClamp software (Molecular Devices), and digitized at 1 kHz using Digidata 1332A digitizer (MDS Analytical Technologies).

Oocytes were impaled with borosilicate recording microelectrodes (0.3-3.0 MΩ resistance) backfilled with 3 M KCl. Sodium currents were recorded using a bath solution containing the following, in millimolar: 96, NaCl; 1, CaCl₂; 1, MgCl₂; 2, KCl; and 5, HEPES (pH 7.5 with NaOH), supplemented with antibiotics (50 μg ml⁻¹ gentamycin, 100 IU ml⁻¹ penicillin and 100 μg ml⁻¹ streptomycin) and 2.5 mM sodium pyruvate.

Sxph responses were measured using Sxph or Sxph mutants purified as described above. Following recording of channel behavior in the absence of toxin, 100 nM STX was applied to achieve ˜90% block. Sxph was then added directly to a 1 mL recording chamber containing the toxin to the desired concentration. For all [Sxph]:[STX] ratios, the concentration of the stock Sxph solution added to the chamber was adjusted so that the volume of the added Sxph solution was less than 1% of the total volume of the recording chamber.

All toxin effects were assessed with 60-ms depolarization steps from −120 to 0 mV with a holding potential of −120 mV and a sweep-to-sweep duration of 10 s.

Recordings were conducted at room temperature (23±2° C.). Leak currents were subtracted using a P/4 protocol during data acquisition. Data Analysis was performed using Clampfit 10.6 (Axon Instruments) and SigmaPlot (Systat Software).

### F-STX Synthesis

All reagents were obtained commercially unless otherwise noted. N,N-Dimethylformamide (DMF) was passed through two columns of activated alumina prior to use. High-performance liquid chromatography-grade CH₃CN and H₂O were obtained from commercial suppliers. Semi-preparative high-performance liquid chromatography (HPLC) was performed on a Varian ProStar model 210. A high-resolution mass spectrum of F-STX was obtained from the Vincent Coates Foundation Mass Spectrometry Laboratory at Stanford University. The sample was analyzed with HESI-MS by direct injection onto Waters Acquity UPLC and a Thermo Fisher Orbitrap Exploris™ 240 mass spectrometer scanning m/z 100-1000. F-STX was quantified by ¹H NMR spectroscopy on a Varian Inova 600 MHz NMR instrument using distilled DMF as an internal standard. A relaxation delay (dl) of 20 s and an acquisition time (at) of 10 s were used for spectral acquisition. The concentration of F-STX was determined by integration of ¹H signals corresponding to F-STX and a fixed concentration of the DMF standard.

To an ice-cold solution of saxitoxin-N21-hexylamine (1.4 μmol) in 140 μL of pH 9.5 aqueous bicarbonate buffer (0.2 M aqueous NaHCO₃, adjusted to pH 9.5 with 1 M aqueous NaOH) was added a solution of fluorescein NHS-ester, 6-isomer (2.0 mg, 4.2 mol, 3.0 equiv, Lumiprobe) in 140 μL of DMSO. The reaction flask was stoppered, wrapped in foil, and placed in a sonication bath for 30 s. The reaction mixture was then stirred at room temperature for 4 h. Following this time, the reaction was quenched by the addition of 0.3 mL of 1% aqueous CF₃CO₂H. The reaction mixture was diluted with 1.1 mL of 10 mM aqueous CF₃CO₂H and 0.3 mL of DMSO and filtered through a VWR 0.22 μm PTFE filter. The product was purified by reverse-phase HPLC (Silicycle SiliaChrom dt C18, 5 μm, 10×250 mm column, eluting with a gradient flow of 10→40% CH₃CN in 10 mM aqueous CF₃CO₂H over 40 min, 214 nm UV detection). At a flow rate of 4 mL/min, F-STX had retention time of 31.00 min and was isolated as a dark yellow powder following lyophilization (1.08 μmol, 77%, ¹H NMR quantitation).

¹H NMR (600 MHz, D₂O) δ 8.05 (d, J=8.1 Hz, 1H), 7.94 (d, J=8.9 Hz, 1H), 7.48 (s, 1H), 6.95 (d, J=9.0 Hz, 2H), 6.79 (s, 2H), 6.67 (dt, J=9.1, 2.2 Hz, 2H), 4.60 (d, J=1.2 Hz, 1H), 4.09-4.05 (m, 1H), 3.89 (dd, J=11.6, 5.2 Hz, 1H), 3.70 (dt, J=10.1, 5.5 Hz, 1H), 3.64 (dd, J=8.7, 5.4 Hz, 1H), 3.47-3.42 (m, 1H), 3.27 (t, J=6.6 Hz, 2H), 2.97-2.89 (m, 2H), 2.36-2.33 (m 1H), 2.30-2.24 (m, 1H), 1.48-1.45 (m, 2H), 1.32-1.29 (m, 2H), 1.25-1.21 (m, 4H) ppm. HRMS (ESI⁺) calcd for C₃₇H₄₁N₈O₁₀, 757.2940; found 757.2918 (MJ)

### Example 2: Protocol for Testing In Vivo Efficacy of Sxphs as Toxin Antidotes

Saxitoxin (STX) is one of the most lethal natural paralytic neurotoxins due to its ability to stop electrical signals in nerves by inhibiting the action of proteins known as voltage-gated sodium channels (Na_{V}s). Because of its extreme potency and lethality, STX is classified as a chemical weapon. STX and related toxins are produced by bacteria and plankton associated with oceanic red tides and cause paralytic shellfish poisoning (PSP). There are no antidotes that can be administered to mitigate STX poisoning. Remarkably, some animals, particularly select frog species, are naturally resistant to STX poisoning. How these creatures evade the lethal effects of STX remains unknown.

A leading candidate is a class of soluble high-affinity STX binding proteins known as saxiphilins (Sxphs). These and related proteins are thought to act as ‘toxin sponges’ that sequester and my help eliminate STX, thereby protecting the frog nervous system from STX inhibition. The experiments outlined below are directed at testing whether high-affinity STX binding proteins, Sxphs, can be used as countermeasures against STX poisoning. We pursue two approaches that will test the ability of Sxphs and related proteins to reverse STX poisoning in mouse models.

Individual trials will use 6-10 mice for each condition tested. These values are based on previous studies used to test anti-STX and anti-TTX antibodies (Davio, Fukiya and Matsumura). Because there are reports of differential effects on male vs. female mice, all trials will include equal numbers of males and females in each test condition.

Initial studies will need 60 animals (6 different conditions, 10 mice each; STX, Sham, Sxph, Sxph/STX, TTX, TTX/Sxph). We expect to need to test various rations of Sxph/STX as well as Sxph mutants having enhanced or reduced STX binding capacity. For those experiments, we will have to have controls (STX, Sxph, Sham) that will each require 10 mice. These numbers are based on similar published experiments testing the efficacy of anti-STX antisera (Davio); 10 mice per condition) and anti-TTX IgG ((Fukiya and Matsumura); 12 mice per condition).

Our objective is to test whether saxiphilins (Sxphs) or similar high affinity STX binding proteins can be used as an antidote for STX poisoning. Using in vitro models, we have recently shown that bullfrog (Rana castesbeiana) Sxph (RcSxph) can effectively rescue voltage-gated sodium channels from STX block (Abderemane-Ali et al.). This in vitro demonstration is a key proof-of-concept that underpins the proposed animal studies. Even though we can show that Sxph application to a cell expressing a voltage-gated sodium channel can rescue the channel from STX block, it is impossible to know if Sxph can do the same in a complex physiological setting. Hence, in order to test whether Sxphs can be developed as antidotes for STX poisoning, we have to use an animal system.

Mice (Mus musculus) are the best species for testing reversal of STX effects. Their sensitivity to STX poisoning is the bases for the gold-standard assay that is used to test shellfish for human consumption to ensure that the shellfish are not contaminated with STX (AOAC). Prior work testing the efficacy of rabbit STX antisera (Davio, Kaufman et al.) and a donkey anti-STX antibody (Benton et al.) conducted experiments using a mouse model system. As these are the only studies in the literature for which we have a comparison, we think that the mouse model is the best system for the proposed experiments.

These experiments are designed to test whether saxiphilins, or other saxitoxin binding molecules, can be used as medical countermeasures against saxitoxin poisoning. Currently, no such treatments exist.

This protocol follows the basic intraperitoneal (i.p.) injection protocol outlined by Munday et al., Based on Munday et al., 30 nmoles/Kg of STX delivered i.p. is sufficient to achieve 100% lethality within 10 minutes. This will be the initial quantity of STX used in our experiments. Based on Finch et al., 70 nM/Kg of TTX delivered i.p. is equivalent to 30 nmoles/Kg STX. This will be the initial quantity of TTX used in our experiments. This is an important control as Sxph does not bind TTX, and TTX and STX work by similar mechanisms of action.

### Experiment #1 (Prophetic)

1) STX, Sxph, TTX, Sxph:STX, or Sxph:TTX mixtures will be prepared in phospho-buffered saline (150 mM NaCl, 10 mM PO4, pH 7.4).

2) Mice will be weighed before treatment.

3) Mice will be injected IP with about 30 nmoles/Kg STX, Sxph, or STX:Sxph mixtures. Initial experiments will aim for 1:1 or 1:2 molar rations of STX:Sxph as these are sufficient to reverse the effects of STX on sodium channels based on our recent work (Abderemane-Ali et al.).

Solutions will be prepared to deliver a 100 μl i.p. injection per animal using a 25-27G needle. Test solutions will be delivered IP as follows:

i) Mice will be restrained manually with the body tilted downward, and the head of the animal tilted back.

ii) Insert needle (25-27G needle size) with bevel facing “up” into the lower right quadrant of the abdomen towards the head at a 30-40° angle to horizontal. Insert needle to the depth in which the entire bevel is within the abdominal cavity.

iii) Deliver solution after aspiration. Only one injection will be delivered per mouse. No pregnant females will be used.

iv) Animals will be monitored following injection to note symptoms, time of onset, and time of death if it occurs. Based on prior work, Munday et al., we expect to see the following symptoms: lethargy, rapid abdominal breathing, immobility, irregular respiration, paralysis, cynanosis. To reduce pain and distress, if any animals having only the toxin administered are experiencing abdominal breathing, they will immediately be euthanized per section N because we can anticipate this animal will die very soon. Animals administered the possible antidote will need to be monitored longer so that we can detect if and when the antidote is able to reverse the effects of the toxin (see below).

v) Data of time to death will be plotted to assess efficacy of Sxph, Sxph/Toxin ratios, and Sxph variant function.

Based on the above references, we expect the animals to survive only ˜10 minutes in the absence of the toxin binding proteins. For animals that have only had the toxin administered, they will be euthanized at 10 minutes if the animals have not died by that point.

Animals receiving control injections (buffer only, toxin binding protein only) will be monitored for the same period. Provided these animals are not showing signs of distress, we will continue the monitoring to 1 hour in order to gauge the effectiveness of the antidote. These animals will be euthanized at the 1 hour time point.

### Experiment #2

We have performed experiments to test whether high-affinity STX binding proteins, Sxphs, can be used as countermeasures against STX poisoning. This research is an essential step towards developing new Sxph-inspired biologic countermeasures that could be used to mitigate STX poisoning as caused by PSP or by an STX attack in humans. Our initial data show that RcSxph protects mice from STX poisoning.

We conducted the experiment using 20 female CD-1 mice. Mice received intraperitoneal (IP) injections of the following solutions and were observed for up to one hour before being euthanized. Toxin amounts were chosen to show clear symptoms within the first 10 minutes of the observation period.

1) RcSxph, STX, and TTX, and RcSxph:STX, or RcSxph:TTX mixtures were prepared in phospho-buffered saline (150 mM NaCl, 10 mM PO4, pH 7.4).

TABLE 3

| Injected Solution | n |
|---|---|
| Phospho buffered saline (PBS) | 3 |
| RcSxph | 3 |
| STX | 5 |
| RcSxph:STX (2:1 molar ratio) | 3 |
| TTX | 3 |
| RcSxph:TTX (1:1.2 molar ratio) | 3 |
| | Total 20 |

2) Mice were weighed before treatment.

3) Mice were injected IP with PBD, 30 nmoles/Kg STX, 70 nmoles/Kg TTX, and 60 nmoles/Kg RcSxph, and RcSxph:STX, and RcSxph:TTX mixtures. Initial experiments aimed for 2:1 molar rations of RcSxph:STX and RcSxph:TTX as these are sufficient to observe the effects of STX and TTX on sodium channels based on our recent work (Abderemane-Ali et al.).

Solutions were prepared to deliver a 100 μl i.p. injection per animal using a 25-27G needle. Test solutions were delivered IP as follows:

i) Mice were restrained manually with the body tilted downward, and the head of the animal tilted back.

ii) Insert needle (25-27G needle size) with bevel facing “up” into the lower right quadrant of the abdomen towards the head at a 30-40° angle to horizontal. The needle was inserted to the depth in which the entire bevel is within the abdominal cavity.

iii) Deliver solution after aspiration. Only one injection was delivered per mouse. No pregnant females were used.

iv) Animals were monitored following injection to note symptoms, time of onset, and time of death if it occurs. Based on prior work, Munday et al., we expected to see the following symptoms: lethargy, rapid abdominal breathing, immobility, irregular respiration, paralysis, cynanosis. To reduce pain and distress, if any animals having only the toxin administered were experiencing abdominal breathing, they were immediately euthanized per section N because we can anticipate this animal die very soon. Animals administered the possible antidote were needed to be monitored longer so that we can detect if and when the antidote is able to reverse the effects of the toxin (see below).

v) Data of time to death was plotted to assess efficacy of Sxph, Sxph/Toxin ratios, and Sxph variant function.

On FIG. 18, dots indicate mice having no symptoms. X's indicate mice showing symptoms of STX or TTX poisoning. Box (RcSxph:STX=2:1 molar ratio) highlights the key rescue experiment that provides the first evidence that RcSxph can act as an STX countermeasure.

The results of this experiments (FIG. 18) show two key findings:

(1) IP injection of RcSxph alone has no detectable adverse effects.

(2) a 2:1 molar ratio of RcSxph:STX is sufficient to protect mice completely from STX intoxication. Mice receiving the 2:1 molar mixture of RcSxph:STX had behaviors that were indistinguishable from PBS control, or RcSxph injected mice. This contrasted strongly with the STX group, who displayed multiple symptoms of STX poisoning (lethargy, tremors, immobility). Consistent with its inability to bind TTX, RcSxph was unable to mitigate TTX poisoning.

This data provide evidence that Saxiphilins can be used as a basis for anti-STX medical countermeasures.

Each of the following references is incorporated by reference in their entireties.

### REFERENCES

Abderemane-Ali F. et al., Evidence that toxin resistance in poison birds and frogs is not rooted in sodium channel mutations and may rely on “toxin sponge” proteins. J Gen Physiol 153, (2021).
Adams, P D et al., PHENIX: a comprehensive Python-based system for macromolecular structure solution. Acta Crystallogr D Biol Crystallogr 66, 213-221 (2010).
Anderson, D M et al., Evidence for massive and recurrent toxic blooms of Alexandrium catenella in the Alaskan Arctic. Proc Natl Acad Sci USA 118, (2021).
Andresen, B M, J. Du Bois, De novo synthesis of modified saxitoxins for sodium ion channel study. J Am Chem Soc 131, 12524-12525 (2009).
AOAC, in Official Methods of Analysis. (Arlington, VA, 1999), vol. 1.
Arrigoni, D. L. Minor, Jr., Global versus local mechanisms of temperature sensing in ion channels. Pflugers Arch 470, 733-744 (2018).
Becktel, W J, J. A. Schellman, Protein stability curves. Biopolymers 26, 1859-1877 (1987).
Benton, B J. V. R. Rivera, J. F. Hewetson, F. C. Chang, Reversal of saxitoxin-induced cardiorespiratory failure by a burro-raised alpha-STX antibody and oxygen therapy. Toxicol Appl Pharmacol 124, 39-51 (1994).
Caty, S N, et al., Molecular physiology of chemical defenses in a poison frog. J Exp Biol 222,
(2019).
Clackson, T., J. A. Wells, A hot spot of binding energy in a hormone-receptor interface. Science 267, 383-386. (1995).
Cowtan, K, General quadratic functions in real and reciprocal space and their application to likelihood phasing. Acta Crystallogr D Biol Crystallogr 56, 1612-1621 (2000).
Cowtan, K, The Buccaneer software for automated model building. 1. Tracing protein chains. Acta Crystallogr D Biol Crystallogr 62, 1002-1011 (2006).
Cowtan, K, Fitting molecular fragments into electron density. Acta Crystallogr D Biol Crystallogr 64, 83-89 (2008).
Davio, S R Neutralization of saxitoxin by anti-saxitoxin rabbit serum. Toxicon 23, 669-675 (1985).
Doyle, D D, M. Wong, J. Tanaka, L. Barr, Saxitoxin binding sites in frog-myocardial cytosol. Science 215, 1117-1119 (1982).
Duran-Riveroll, L M, A. D. Cembella, Guanidinium Toxins and Their Interactions with Voltage-Gated Sodium Ion Channels. Mar Drugs 15, (2017).
Edwards, R J, et al., Draft genome assembly of the invasive cane toad, Rhinella marina. Gigascience 7, (2018).
Emsley, P, K. Cowtan, Coot: model-building tools for molecular graphics. Acta Crystallogr D Biol Crystallogr 60, 2126-2132 (2004).
Evans, P R, G. N. Murshudov, How good are my data and what is the resolution? Acta Crystallogr D Biol Crystallogr 69, 1204-1214 (2013).
Feng, Y J, et al., Phylogenomics reveals rapid, simultaneous diversification of three major clades of Gondwanan frogs at the Cretaceous-Paleogene boundary. Proc Natl Acad Sci USA 114, E5864-E5870 (2017).
Finch, S C, M. J. Boundy, D. T. Harwood, The Acute Toxicity of Tetrodotoxin and Tetrodotoxin(−)Saxitoxin Mixtures to Mice by Various Routes of Administration. Toxins (Basel) 10, (2018).
Fukiya, S, K. Matsumura, Active and passive immunization for tetrodotoxin in mice. Toxicon 30, 1631-1634 (1992).
Hansen, S, et al., Regulation of the Escherichia coli HipBA toxin-antitoxin system by proteolysis. PLoS One 7, e39185 (2012).
Heinemann, S H, H. Terlau, K. Imoto, Molecular basis for pharmacological differences between brain and cardiac sodium channels. Pflugers Arch 422, 90-92 (1992).
Hille, B, Ion Channels of Excitable Membranes. (Sinauer Associates, Inc., Sunderland, M A, ed. 3rd, 2001).
Huang, X, A. Aulabaugh, Application of Fluorescence Polarization in HTS Assays. Methods Mol Biol 1439, 115-130 (2016).
Huynh, K, C. L. Partch, Analysis of protein stability and ligand interactions by thermal shift assay. Curr Protoc Protein Sci 79, 28 29 21-14 (2015).
Infield, D T et al., Cation-pi Interactions and their Functional Roles in Membrane Proteins. J Mol Biol 433, 167035 (2021).
Kabsch, Xds. Acta Crystallogr D Biol Crystallogr 66, 125-132 (2010).
Kao, C Y, F. A. Fuhrman, Differentiation of Actions of Tetrodotoxin and Saxitoxin. Toxicon 5, 25-34 (1967).
Kaufman, B., D. C. Wright, W. R. Ballou, D. Monheit, Protection against tetrodotoxin and saxitoxin intoxication by a cross-protective rabbit anti-tetrodotoxin antiserum. Toxicon 29, 581-587 (1991).
Lambert, H. Perri, P. J. Halbrooks, A. B. Mason, Evolution of the transferrin family: conservation of residues associated with iron and anion binding. Comp Biochem Physiol B Biochem Mol Biol 142, 129-141 (2005).
Leffler, A, R. I. Herzog, S. D. Dib-Hajj, S. G. Waxman, T. R. Cummins, Pharmacological properties of neuronal TTX-resistant sodium channels and the role of a critical serine pore residue. Pflugers Arch 451, 454-463 (2005).
Lenarčič, B, et al., Saxiphilin, a saxitoxin-binding protein with two thyroglobulin type 1 domains, is an inhibitor of papain-like cysteine proteinases. J Biol Chem 275, 15572-15577 (2000).
Li, L. Llewellyn, E. Moczydlowski, Biochemical and Immunochemical Comparison of Saxiphilin and Transferrin, 2 Structurally Related Plasma-Proteins from Rana-Catesbeiana. Molecular Pharmacology 44, 742-748 (1993).
Lin, H, et al., Neutralizing effect of hemolymph from the shore crab, Thalamita crenata, on paralytic shellfish toxins. Toxicon 99, 51-57 (2015).
Llewellyn, L E, E. G. Moczydlowski, Characterization of saxitoxin binding to saxiphilin, a relative of the transferrin family that displays pH-dependent ligand binding. Biochemistry 33, 12312-12322 (1994).
Llewellyn, L E, P. M. Bell, E. G. Moczydlowski, Phylogenetic survey of soluble saxitoxin-binding activity in pursuit of the function and molecular evolution of saxiphilin, a relative of transferrin. Proc Biol Sci 264, 891-902 (1997).
Llewellyn, L E, Saxitoxin, a toxic marine natural product that targets a multitude of receptors. Nat Prod Rep 23, 200-222 (2006).
Lukowski, A L et al., Biocatalytic Detoxification of Paralytic Shellfish Toxins. ACS Chem Biol 14, 941-948 (2019).
Lukowski, A L, J. Liu, J. Bridwell-Rabb, A. R. H. Narayan, Structural basis for divergent C—H hydroxylation selectivity in two Rieske oxygenases. Nat Commun 11, 2991 (2020).
Mahar, J, G. L. Lukacs, Y. Li, S. Hall, E. Moczydlowski, Pharmacological and biochemical properties of saxiphilin, a soluble saxitoxin-binding protein from the bullfrog (Rana catesbeiana). Toxicon 29, 53-71 (1991).
McGugan, J R et al., Ant and Mite Diversity Drives Toxin Variation in the Little Devil Poison Frog. J Chem Ecol 42, 537-551 (2016).
Morabito, M A, E. Moczydlowski, Molecular cloning of bullfrog saxiphilin: a unique relative of the transferrin family that binds saxitoxin. Proc Natl Acad Sci USA 91, 2478-2482 (1994).
Morris, R J et al., Breaking good resolutions with ARP/wARP. J Synchrotron Radiat 11, 56-59 (2004).
Munday, R., K. Thomas, R. Gibbs, C. Murphy, M. A. Quilliam, Acute toxicities of saxitoxin, neosaxitoxin, decarbamoyl saxitoxin and gonyautoxins 1&4 and 2&3 to mice by various routes of administration. Toxicon 76, 77-83 (2013).
N. Collaborative Computational Project, The CCP4 suite: Programs for protein crystallography. Acta Crystallogr D Biol Crystallogr 50, 760-763. (1994).
Niesen, F H, H. Berglund, M. Vedadi, The use of differential scanning fluorimetry to detect ligand interactions that promote protein stability. Nat Protoc 2, 2212-2221 (2007).
O'Connell, L A et al., Rapid toxin sequestration modifies poison frog physiology. J Exp Biol 224, (2021).
Ondrus, A E, et al., Fluorescent saxitoxins for live cell imaging of single voltage-gated sodium ion channels beyond the optical diffraction limit. Chem Biol 19, 902-912 (2012).
Panjikar, S, V. Parthasarathy, V. S. Lamzin, M. S. Weiss, P. A. Tucker, Auto-rickshaw: an automated crystal structure determination platform as an efficient tool for the validation of an X-ray diffraction experiment. Acta Crystallogr D Biol Crystallogr 61, 449-457 (2005).
Prinzmetal, M., H. Sommer, C. D. Leake, The pharmacological action of “mussel poison”. J Pharmacol Exp Ther 46, 63-73 (1932).
Rossi, A M, C. W. Taylor, Analysis of protein-ligand interactions by fluorescence polarization. Nat Protoc 6, 365-387 (2011).
Satin, J. et al., A mutant of TTX-resistant cardiac sodium channels with TTX-sensitive properties. Science 256, 1202-1205 (1992). (Satin et al.)
Shen, H, et al., Structural basis for the modulation of voltage-gated sodium channels by animal toxins. Science 362, (2018).
Shen, H, D. Liu, K. Wu, J. Lei, N. Yan, Structures of human Na_{V}1.7 channel in complex with auxiliary subunits and animal toxins. Science 363, 1303-1308 (2019).
Sivilotti, L, K. Okuse, A. N. Akopian, S. Moss, J. N. Wood, A single serine residue confers tetrodotoxin insensitivity on the rat sensory-neuron-specific sodium channel SNS. FEBS Lett 409, 49-52 (1997).
Su, Z, M. Sheets, H. Ishida, F. Li, W. H. Barry, Saxitoxin blocks L-type ICa. J Pharmacol Exp Ther 308, 324-329 (2004).
Takati, N, D. Mountassif, H. Taleb, K. Lee, M. Blaghen, Purification and partial characterization of paralytic shellfish poison-binding protein from Acanthocardia tuberculatum. Toxicon 50, 311-321 (2007).
Tanaka, J C, D. D. Doyle, L. Barr, Sodium channels in vertebrate hearts. Three types of saxitoxin binding sites in heart. Biochim Biophys Acta 775, 203-214 (1984).
Thomas-Tran, R, J. Du Bois, Mutant cycle analysis with modified saxitoxins reveals specific interactions critical to attaining high-affinity inhibition of hNaV1.7. Proc Natl Acad Sci USA 113, 5856-5861 (2016).
Thottumkara, A P, W. H. Parsons, J. Du Bois, Saxitoxin. Angew Chem Int Ed Engl 53, 5760-5784 (2014).
Velazquez-Campoy, H. Ohtaka, A. Nezami, S. Muzammil, E. Freire, Isothermal titration calorimetry. Curr Protoc Cell Biol Chapter 17, Unit 17 18 (2004).
Wang, J, J. J. Salata, P. B. Bennett, Saxitoxin is a gating modifier of HERG K+ channels. J Gen Physiol 121, 583-598 (2003).
Wells, J A, Additivity of mutational effects in proteins. Biochemistry 29, 8509-8517 (1990).
Wells, J A, Systematic mutational analyses of protein-protein interfaces. Methods Enzymol 202, 390-411 (1991).
Wiese, M, P. M. D'Agostino, T. K. Mihali, M. C. Moffitt, B. A. Neilan, Neurotoxic alkaloids: saxitoxin and its analogs. Mar Drugs 8, 2185-2211 (2010).
Williams, C J et al., MolProbity: More and better reference data for improved all-atom structure validation. Protein Sci 27, 293-315 (2018).
Winn, M D et al., Overview of the CCP4 suite and current developments. Acta Crystallogr D Biol Crystallogr 67, 235-242 (2011).
Yen, T J, M. Lolicato, R. Thomas-Tran, J. Du Bois, D. L. Minor, Jr., Structure of the Saxiphilin:saxitoxin (STX) complex reveals a convergent molecular recognition strategy for paralytic toxins. Sci Adv 5, (2019).
Yotsu-Yamashita, M et al., Purification, characterization, and cDNA cloning of a novel soluble saxitoxin and tetrodotoxin binding protein from plasma of the puffer fish, Fugu pardalis. Eur J Biochem 268, 5937-5946 (2001).
Yotsu-Yamashita, M, H. Yamaki, N. Okoshi, N. Araki, Distribution of homologous proteins to puffer fish saxitoxin and tetrodotoxin binding protein in the plasma of puffer fish and among the tissues of Fugu pardalis examined by Western blot analysis. Toxicon 55, 1119-1124 (2010).
Zhang, Q. Wu, M. Y. Berezin, Fluorescence anisotropy (polarization): from drug screening to precision medicine. Expert Opin Drug Discov 10, 1145-1161 (2015).

1. A method for treating a PSP in a subject in need thereof, the method comprising administering to the subject a therapeutically effective amount of a first amino acid sequence, or a pharmaceutically acceptable salt thereof; wherein the first amino acid sequence comprises at least about 70% sequence identity to X₁X₂X₃X₄X₅X₆X₇X₈X₉X₁₀X₁₁X₁₂X₁₃X₁₄; wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁₁=any amino acid
X₁₂=any amino acid
X₁₃=D
X₁₄=Y, V

2. The method of claim 1, wherein the wherein the first amino acid sequence comprises about 70% sequence identity to X₁X₂X₃X₄X₅X₆X₇X₈X₉X₁₀X₁₁X₁₂X₁₃X₁₄.

3. The method of claim 1, wherein the first amino acid sequence comprises at least about 80% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO:15, or a functional fragment thereof.

4. The method of claim 1, wherein the first amino acid is a functional fragment comprising at least about 75% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, or SEQ ID NO:15.

5. The method of claim 1, wherein the first amino acid is chosen from an amino acid sequence comprising a substitution mutation at amino acid position chosen from: 540, 558, 559, 561, 563, 727, 782, 784, 785, 787, 789, 794 and/or 795, in relation to such amino acid numbers identified in any of SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, or SEQ ID NO: 15.

6. The method of claim 1, wherein the first amino acid comprises at least 90% sequence identity to X₁X₂X₃X₄X₅X₆X₇X₈X₉X₁₀X₁₁X₁₂X₁₃X₁₄;
wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁₁=any amino acid
X₁₂=any amino acid
X₁₃=D
X₁₄=Y, V

7. The method of claim 1, wherein the first amino acid sequence comprises about 70% sequence identity to an amino acid comprising contiguous amino acids with Formula: X_{B}−[from about 53 to about 56 amino acids]−[X₁X₂X₃X₄X₅X₆X₇X₈X₉X₁₀X₁₁X₁₂X₁₃X₁₄]; wherein
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V

8. The method of claim 7, wherein the first amino acid sequence comprises at least about 75% sequence identity to an amino acid with contiguous amino acids of Formula III: [X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein
X₁ₐ=Y
X₂ₐ=any amino acid
X₃ₐ=an amino acid
X₄ₐ=F
X₅ₐ=any amino acid
X₆ₐ=S or G
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V

9. The method of claim 8, wherein the first amino acid sequence comprises at least about 75% sequence identity to an amino acid with contiguous amino acids of Formula IV: [X_{A}]−[from about 15 to about 20 amino acids]−[X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein
X_{A}=D or E
X₁ₐ=Y
X₂ₐ=any amino acid
X₃ₐ=an amino acid
X₄ₐ=F
X₅ₐ=any amino acid
X₆ₐ=S or G
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V

10. The method of claim 1, wherein the subject is a mammal.

11. The method of claim 1, wherein the subject has been diagnosed with a need for treatment of the PSP prior to the administering step.

12. The method of claim 1, wherein the effective amount is a prophylactically effective amount.

13. A method for neutralizing a toxin comprising administering to the subject a therapeutically effective amount of an amino acid sequence comprising at least about 80% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO:15, a functional fragment thereof or a pharmaceutically acceptable salt thereof.

14. The method of claim 13, wherein the first amino acid is a functional fragment comprising at least about 75% sequence identity to SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, or SEQ ID NO:15, or a pharmaceutically accept table salt thereof.

15. The method of claim 13, wherein the first amino acid is chosen from an amino acid sequence comprising a substitution mutation at amino acid position chosen from one or a combination of: 540, 558, 559, 561, 563, 727, 782, 784, 785, 787, 789, 794 and/or 795, in relation to such amino acid positions identified in any of SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, or SEQ ID NO:15.

16. A method for neutralizing a toxin comprising administering to the subject a therapeutically effective amount of an amino acid sequence comprising at least about 75% sequence identity to X₁X₂X₃X₄X₅X₆X₇X₈X₉X₁₀X₁₁X₁₂X₁₃X₁₄;
wherein X₁=I, V, F, L
X₂=any amino acid
X₃=F
X₄=D
X₅=any amino acid
X₆=M, Q, I
X₇=any amino acid
X₈=R, K
X₉=any amino acid
X₁₀=any amino acid
X₁=any amino acid
X₁₂=any amino acid
X₁₃=D
X₁₄=Y, V,
or a pharmaceutically acceptable salt thereof.

17. The method of claim 16, wherein the first amino acid sequence comprises about 70% sequence identity to an amino acid comprising contiguous amino acids with Formula: X_{B}−[from about 53 to about 56 amino acids]−[X₁X₂X₃X₄X₅X₆X₇X₈X₉X₁₀X₁₁X₁₂X₁₃X₁₄]; wherein
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₁₃=D
X₁₄=Y or V, or a pharmaceutically salt thereof.

18. The method of claim 16, wherein the first amino acid sequence comprises at least about 75% sequence identity to an amino acid with contiguous amino acids of Formula III: [X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein
X₁ₐ=Y
X₂ₐ=any amino acid
X₃ₐ=an amino acid
X₄ₐ=F
X₅ₐ=any amino acid
X₆ₐ=S or G
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁₁=any amino acid
X₁₂=any amino acid
X₃=D
X₁₄=Y or V,
or a pharmaceutically acceptable salt thereof.

19. The method of claim 16, wherein the first amino acid sequence comprises at least about 75% sequence identity to an amino acid with contiguous amino acids of Formula IV: [X_{A}]−[from about 15 to about 20 amino acids]−[X₁ₐX₂ₐX₃ₐX₄ₐX₅ₐX₆ₐ]−[from about 161 to about 164 amino acids]−X_{B}−[from about 53 to about 56 amino acids]−[X₁X₂X₃X₄X₅X₆X₇X₈X₉X₁₀X₁₁X₁₂X₁₃X₁₄]; wherein
X_{A}=D or E
X₁ₐ=Y
X₂ₐ=any amino acid
X₃ₐ=an amino acid
X₄ₐ=F
X₅ₐ=any amino acid
X₆ₐ=S or G
X_{B}=N or P
X₁=I, V, F, or L
X₂=any amino acid
X₃=F
X₄=D
X₅=any amino acid
X₆=M, Q, or I
X₇=any amino acid
X₈=R or K
X₉=any amino acid
X₁₀=any amino acid
X₁=any amino acid
X₁₂=any amino acid
X₁₃=D
X₁₄=Y or V;
or a pharmaceutically acceptable salt thereof.

20. The method of claim 16, wherein the subject is a mammal.

21. The method of claim 16, wherein the subject has been diagnosed with a need for treatment of the PSP prior to the administering step.

22. The method of claim 16, wherein the compound is free of an amino acid that is SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO:15 or a pharmaceutically acceptable salt thereof.

23. A pharmaceutical composition comprising a therapeutically effective amount of a saxiphilin; and a pharmaceutically acceptable carrier.

24. The pharmaceutical composition of claim 23, wherein the saxiphilin is an amino acid sequence comprising at least 80% sequence identity to SEQ ID NO:1 and comprising at least one of the following amino acid substitutions in SEQ ID NO:1: an alanine for the isoleucine at position 782; an alanine for the tyrosine at position 558; an isoleucine for the tyrosine at position 558; an asparagine for the aspartic acid at position 785; an alanine for the lysine at position 789; an alanine for the threonine at position 563; a phenylalanine for a tyrosine at position 558; a glutamic acid for the glutamine at position 787; an alanine for a tyrosine at position 795; an alanine for the glutamine at position 787; a tyrosine for the phenylalanine at position 784; a phenylalanine for the isoleucine at position 782; an alanine for the phenylalanine at position 561; an alanine for the aspartic acid at position 785; a leucine for the phenylalanine at position 784; an alanine for the proline at position 727; an aspartic acid for the glutamic acid at position 540; an alanine for the phenylalanine at position 784; a glutamic acid for the aspartic acid at position 794; a cysteine for the phenylalanine at position 784; a asparagine for an aspartic acid at position 794; a serine for the phenylalanine at position 784; a glutamine for the glutamic acid at position 540; an alanine for the aspartic acid at position 794; an alanine for the glutamic acid at position 540; or a pharmaceutically acceptable salt thereof.

25. (canceled)

26. (canceled)

27. (canceled)

28. The pharmaceutical composition of claim 23, wherein the composition is free of SEQ ID NO:1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO:13, SEQ ID NO:14, SEQ ID NO:15 or a pharmaceutically acceptable salt thereof

29. A kit comprising the pharmaceutical composition of claim 23, and one or more selected from:
a. an agent known for treating a PSP;
b. instructions for treating a PSP; and
c. instructions for administering the pharmaceutical composition.
